(19) United States
(12) Patent Application Publication
(10) Pub. No.: US 2011/0065086 A1
(43) Pub. Date: Mar. 17, 2011

(54) Methods of producing homogeneous plastic-adherent aptamer-magnetic bead-fluorophore and other sandwich assays

(75) Inventor: John G. Bruno, San Antonio, TX (US)

(73) Assignee: OTC Biotechnologies, LLC

(21) Appl. No.: 12/378,515

(22) Filed: Feb. 17, 2009

Related U.S. Application Data
(60) Provisional application No. 61/066,506, filed on Feb. 21, 2008.
(60) Provisional application No. 61/132,147, filed on Jun. 16, 2008.

(51) Int. Cl. C12Q 1/70; C12Q 1/68; G01N 27/26

(52) U.S. Cl. 435/5; 435/6; 205/792

(57) ABSTRACT

Methods are described for assembly of DNA aptamer-magnetic bead (“MB”) conjugate plus aptamer-quantum dot (“QD”) aptamer-fluorescent nanoparticle or other aptamer-fluorophore, aptamer-chemiluminescent reporter, aptamer-radioisotope or other aptamer-reporter conjugate sandwich assays that enable adherence to glass, polystyrene and other plastics. Adherence to glass or plastics enables detection of surface-concentrated partitioning of fluorescence versus background (bulk solution) fluorescence in one step (without a wash step) even when the external magnetic field for concentrating the assay is removed. This assay format enables rapid, one-step (homogeneous) assays for a variety of analytes without wash steps that do not sacrifice sensitivity.

This application is based upon and claims priority from U.S. Provisional application Ser. Nos. 61/066,506 and 61/132,147, which are incorporated herein by reference.

### BACKGROUND OF THE INVENTION

1. Field of the Invention

The present invention relates to the field of aptamer- and nucleic acid-based diagnostics. More particularly, it relates to methods for the production and use of self-assembling DNA aptamer-magnetic bead (“MB”) conjugate combined with aptamer-quantum dot (“QD”) or other aptamer-fluorophore conjugate sandwich assays that naturally adhere to glass and certain plastics such as polystyrene (or derivatives thereof) to enable one-step (homogeneous) tests without a wash step even after an external magnetic field is removed. Conjugation of aptamers to the MBs or QDs and other fluorophores may be accomplished by simple chemical coupling reactions through bifunctional linkers, or key functional groups such as aldehydes, carbodiimides, carboxyls, N-hydroxy-succinimide (“NHS”) esters, N-oxy-succinimide (“NOS”) esters, thiols, etc. or via biotin-avidin, histidine-Nickel, or other high affinity linkage systems. This one-step, washless assay format has numerous applications for sensitive detection of foodborne pathogens on and in meats, poultry, serous fluids, dairy products, fruits, vegetables, and other food matrices. The assay is also applicable to environmental analyses in soil or muddy water samples and clinical and veterinary diagnostics performed directly on whole blood, urine, saliva or other body fluids with or without sample dilution, but without a wash step. The typical wash step involves purification by removal of unwanted materials contributing to background fluorescence.

2. Background Information

The most desirable of all diagnostic assay strategies are rapid one-step “bind and detect” or “homogeneous” assays that do not require a wash step and yet do not sacrifice a significant degree of sensitivity. Examples of successful one-step assay strategies include fluorescence polarization (“FP”) and fluorescence resonance energy transfer (“FRET”)-based assays. While both of these formats are popular, they tend to sacrifice sensitivity for speed in obtaining test results. Therefore, FP and FRET assays are typically relegated to clinical diagnostics for certain analytes that exist in relatively high concentrations (micro to milliMolar ranges) in blood, urine, or other body fluids. For analytes that exist in much lower concentrations, multi-step assays such as enzyme-linked immunosorbent assays (“ELISA”), radioimmunoassays (“RIA”) and other sandwich-formatted assays such as immunomagnetic-electrochemiluminescence (“IM-ECL”) assays are required to detect nanogram, nanoMolar or lower amounts of various target analytes. Typically, these types of sandwich assays will require one or more wash steps, thereby slowing their execution speed. Wash steps will be known to one skilled in the art as a generally necessary step to remove unwanted materials (besides the detected target analyte) to prevent high background fluorescence signals. Eliminating the need for a wash step is found to be desirable in the present invention because it can enhance the speed and accuracy of many assays.

DNA is well known to adhere to some glass surfaces especially if the surface is charged by rubbing. This principle is used in the electrostatic collection of genomic DNA from cell lysates which is known as “spooling” of DNA with a charged glass rod. Similarly, Allemand et al., Bensimon et al., Buck and Andrews, Dudley et al., Klein et al., Labit et al., Michalet et al., Moscoso et al., and Torres et al. teach adherence of DNA, bacteria, biofilms, and other materials to polystyrene by electrostatic and hydrophobic or other weak forces. However, Allemand et al., Bensimon et al., and Klein et al. emphasize that DNA is far more likely to bind to polystyrene and other plastics at its free 3′ or 5′ ends than in the mid-regions and that such binding is not instantaneous (requires one or more minutes of residence time for DNA to bind to plastic) and is pH-dependent with optimal pH for binding being acidic (at an approximate pH of 5.5, which is below the range of most biological assays). Bensimon et al. (1994) have even suggested that DNA may couple covalently to polystyrene by electrophilic addition of 5′ or 3′ phosphate ends (in their phosphoric acid forms) to the pi double bonds of the styrene rings or free unpolymerized alkene ends of polystyrene fibers. Such covalent bonding of DNA aptamers to polystyrene would explain the very stable and long-lasting adherence of assay materials observed and reported herein and by Bruno et al. (2008) for their Campylobacter assay.

While some species of bacteria can bind to plastics and glass, not all species can form such adherent biofilms. In the presently described assays, attachment of the assay components (DNA aptamers, MBs and QDs) have occurred in the presence and absence of target bacteria. Conversely, immunomagnetic (“antibody-MB”) sandwich assays do not adhere to polystyrene very well at neutral or acidic pH, presumably because protein antibodies do not adhere well to plastic or glass materials at neutral or acidic pH. Proteins such as antibodies are well known to adhere to polystyrene microtiter plate wells at alkaline pH values as in the popular ELISA test formats. However, the pH for adherence of antibodies and proteins in ELISA assays is typically 8.0-9.5 and clearly not acidic as in the presently described DNA-adherent assays. Therefore, the DNA aptamer is considered to be the key component which enables adherence to polystyrene or glass or derivatives thereof and thus enables one-step washless assays. While some species of bacteria may contribute to overall adherence to the inner face of a cuvette, the DNA aptamer component appears sufficient to enable adherence of the aforementioned assays in the magnetized region because assay components (aptamer-MB conjugates) will adhere to plastic and glass even in the absence of captured bacterial cells.

### RELATED LITERATURE

Allemand J. F., et al. pH-dependent specific binding and combing of DNA. Biophys. J. 73:2064-2070, 1997.
Bensimon A., et al. Alignment and sensitive detection of DNA by a moving interface. Science. 265:2096-2098, 1994.
Bensimon D., et al. Stretching DNA with a receding meniscus: experiments and models. Phys. Rev. Lett. 74:4754-4757, 1995.
Bruno J. G., Phillips T., Carrillo M. P., Crowell R. Plastic-adherent DNA aptamer-magnetic bead and quantum dot sandwich assay for Campylobacter detection. J. Fluorescence. In Press, 2008.
Bruno J. G., Carrillo M. P., Phillips T., Crowell R. Initial development of competitive FRET-aptamer assays for monitoring bone metabolism. J. Clin. Ligand Assay. In Press, 2008.
Bruno J. G., Carrillo M. P., Crowell R. Preliminary development of DNA aptamer-Fc conjugate opsonins. J. Biomedical Materials Research-Part A, In Press, 2008.
Bruno J. G., Carrillo M. P., Phillips T. In vitro antibacterial effects of anti-lipopolysaccharide DNA aptamer-C1qrs complexes. Folia Microbiologica. 53:295-302, 2008.
Bruno J. G., Carrillo M. P., Phillips T., King B. Development of DNA aptamers for cytochemical detection of acetylcholine. In Vitro Cell. Develop. Biol.—Animal. 44:63-72, 2008.
Bruno J. G., Carrillo M. P., Phillips T. Development of DNA aptamers to a Foot-and-Mouth Disease peptide for competitive FRET-based detection. J. Biomolecular Techniques. 19:109-115, 2008.
Bruno J. G., Carrillo M. P., Phillips T. Effects of immobilization chemistry on enzyme-linked aptamer assays for Leishmania surface antigens. J. Clinical Ligand Assay. 30:37-43, 2007.
Bruno J. G., Francis K., Ikanovic, M., et al. Reovirus detection using immunomagnetic-fluorescent nanoparticle sandwich assays. J. Bionanoscience. 1:84-89, 2007.
Buck J. W. and Andrews J. H. Localized, positive charge mediates adhesion of Rhodosporidium torulides to barley leaves and polystyrene. Appl. Environ. Microbiol. 65:2179-2183, 1999.
Dudley E. G., et al. An Inch plasmid contributes to the adherence of the atypical enteroaggregative Escherichia coli strain C1096 to cultured cells and abiotic surfaces. Infect. Immun. 74:2102-2114, 2006.
Dwarakanath S., Satyanarayana S., Bruno J. G., et al. Ultra sensitive fluorescent nanoparticle-based binding assays for foodborne and waterborne pathogens of clinical interest. J. Clinical Ligand Assay. 29:136-142, 2006.
Ikanovic M., Rudzinski W. E., Bruno J. G., Dwarakanath S., et al. Fluorescence assay based on aptamer-quantum dot binding to Bacillus thuringiensis spores. J. Fluorescence. 17:193-199, 2007.
shi S. et al. Selection, characterization, and application of DNA aptamers for the capture and detection of Salmonella enterica serovars. Molec. Cell Probes. In Press, 2008.
Klein D. C. G., et al. Ordered stretching of single molecules of deoxyribose nucleic acid between microfabricated polystyrene lines. Appl. Phys. Lett. 78:2396-2398, 2001.
Labit H., et al. A simple and optimized method of producing silanized surfaces for FISH and replication mapping on combed DNA fibers. BioTechniques. 45:649-658, 2008.
Michalet X., et al. Dynamic molecular combing: stretching the whole human genome for high-resolution studies. Science. 277:1518-1523, 1997.
Moscoso M. et al. Biofilm formation by Streptococcus pneumoniae: Role of choline extracellular DNA, and capsular polysaccharide in microbial accretion. J. Bacteriol. 188:7785-7795, 2006.
Quast B. A compact, handheld laboratory fluorometer. American Biotechnol Lab 18:68, 2001.
Torres A. G., et al. Differential binding of Escherichia coli O157:H7 to alfalfa, human epithelial cells, and plastic is mediated by a variety of surface structures. Appl. Environ. Microbiol. 71:8008-8015, 2005.

### SUMMARY OF THE INVENTION

Herein is described a new type of aptamer-MB-aptamer-QD sandwich assay and its derivative formats with variations in the fluorophore component that can be accomplished in one-step, obviating a wash step, by collecting the MBs with a strong external magnetic field onto a glass, polystyrene, other plastic or coated surface such as the inner face of a cuvette. Collection of the MBs and all attached assay components, including DNA aptamers, MBs, fluorophores and the captured analytes, into a small area on the plastic surface thereby focuses fluorescence intensity of the assay due to capture of the analyte in a thin planar area of adherence. Thus, when the adherent material is illuminated even in nearly opaque matrices such as foods or blood, the fluorescence can be detected with ultra sensitivity over background autofluorescence from the bulk solution due to partitioning and concentrating of the assay materials and captured analytes to the area of adherence. Fluorescence from uncaptured aptamer-QD or aptamer-fluorophore conjugates in the bulk solution contributes to background fluorescence, but its contribution to the total fluorescence signal is greatly minimized because it is not concentrated to the area of assay adherence. Any aptamer-QD or aptamer-fluorophore conjugates that do not bind the analyte and aptamer-MB conjugates will not be pulled toward the plastic surface nor adhere to the surface significantly and will not contribute significantly to the detection signal, but will contribute to the much weaker background fluorescence “noise” in the bulk solution. The combination of high aptamer affinity, the MBs ability to be concentrated in a defined area, and the long Stoke's shift of red-emitting QDs (i.e., high energy ultraviolet excitation with emission in the red region of the spectrum above 600 nm) contribute to the ultra sensitive nature of this one-step washless assay format. However, adherence of the assay materials and captured analytes to a small area on a clear plastic or glass surface even when the external magnetic field is removed is the key factor that enables one-step washless detection.

The present invention provides for the assembly of DNA and RNA aptamer-MB conjugates for capture of target analytes with aptamer-QD or other aptamer-fluorophore conjugates. The target analytes are molecules that it is desirable to detect such as, pathogenic bacteria, viruses, parasites, leukocytes, cancer cells, proteins, other macromolecules, toxins, pollutants, drugs, explosives, proteins, viral capsid proteins, viral polymerases, biotoxins such as bacterial toxin, botulinum, cholera, tetanus, staphylococcal enterotoxin, shigatoxins or verotoxins, algal toxins, such as brevetoxin, ciguatoxin, cyanotoxin, or saxitoxin, snake or spider venoms, clinically relevant proteins or portions of proteins (peptides) such as bone marker (e.g., collagen breakdown peptides such as CTx, NTx, OCF, Cathepsin K or its precursor ProCathepsin K, deoxypyridinoline, pyridinoline, lysyl pyridinoline, or hydroxylysyl pyridinoline) cytokines and interleukins, markers of myocardial infarctions (troponin, myoglobin, etc.), kidney disease, antibodies, autoimmune disorders, arthritis, or other clinically relevant macromolecules such as lipopolysaccharides (LPS, endotoxins), and other small molecules (where “small molecules” are defined as being those that are less than 1,000 Daltons) such as with at least two distinct epitopes from a group including the following: pesticides, natural and synthetic amino acids and their derivatives, hydroxylysine, hydroxyproline, histidine, histamine, homocysteine, DOPA, melatonin, nitrotyrosine, short chain proteolysis products, cadaverine, putrescine, polyamines, spermine, spermidine, deoxypyridinoline, pyridinoline, lysyl pyridinoline, or hydroxylysyl pyridinoline, nitrogen bases of DNA or RNA, nucleosides, nucleotides, nucleotide cyclical isoforms, cAMP, cGMP, cellular metabolites, urea, uric acid, pharmaceuticals, therapeutic drugs, vitamins, illegal drugs, narcotics, hallucinogens, gamma-hydroxybutyrate (GHB), cellular mediators, cytokines, chemokines, immune modulators, neural modulators, neurotransmitters such as acetylcholine, inflammatory modulators, prostaglandins, prostaglandin metabolites, nitoaromatic and nitramine explosives, explosive breakdown products (e.g., DNT) or byproducts, quorum sensing molecules such as AHLs, steroids, hormones, and their derivatives.

A fluorophore is a fluorescent component, or functional group, bound to a molecule. A fluorophore can be a dye, a glowing bead, a glowing liposome, a quantum dot (“QD”), a fluorescent or phosphorescent nanoparticle (“NP”), a fluorescent latex particle or microbead, a fluorescent dye molecule, such as fluorescein, carboxyfluorescein and other fluorescein derivatives, rhodamine, and their derivatives, a fluorescence resonance energy transfer (“FRET”) complex such as an intrachain or competitive FRET-aptamer, or any other glowing entity capable of forming a covalent bond with the aptamer. As used herein, “other aptamer-fluorophore conjugates” includes those aptamers having a fluorophore bonded to them, such as, in addition to those listed otherwise herein, aptamer-fluorescent dye conjugates, aptamer-fluorescent microbead conjugates, or aptamer-liposome conjugates containing fluorescent dyes. In the present invention, the fluorophore acts to “report” detection of the target analytes in one rapid and washless step. The only requirement of the target is that it contains two accessible epitopes of the same or different composition and conformation to enable a sandwich assay with capture and reporter aptamer components.

The present invention utilizes a one-step assay format, which can be used for sandwich assay to detect and quantify said target analyte in said bulk solution, as well as fluorescence intensity, time-resolved fluorescence, chemiluminescence, electrical detection, electrochemical detection, electrochemiluminescence, phosphorescence, or radioisotopic detection. The one-step nature of the assay stems from the fact that the assay components capture the analyte and then stick or adhere to the inner surface of the assay substrate, generally expected to be a polystyrene plastic, glass, or other type of cuvette that is transparent or translucent enough so as to allow fluorescent light propagation, in a highly magnetized region for a brief time (5-10 minutes).

More specifically, the one-step nature of the assay stems from the ability, after the application of an external magnetic field, to magnetically separate or partition the assay materials (aptamer-MBs and aptamer-QDs or other aptamer-fluorophore conjugates) from the bulk solution and allow these materials to bind or adhere to a surface such as the inner face of a polystyrene or glass cuvette via the attractive or covalent forces between DNA and some plastics or glass, thereby increasing the signal-to-noise ratio at the surface where the magnet was placed even after the magnet or magnetic field is removed to enable fluorescence detection. The sticking of brightly fluorescing analytes to the inner plane of the cuvette leads to the ability to discriminate the sample's more intense fluorescence from background or target fluorescence from bulk solution or “signal from noise” and to make one-step homogeneous assays possible. Adherence of assay materials to the cuvette constitutes a technique that even allows for detection in dense food samples (e.g. milk, chicken and beef juice, and egg yolk samples).

A typical one-step aptamer-magnetic bead plus aptamer-quantum dot cuvette assay or test will consist of the following two components synthesized and added in any order: 1) One-hundred μg of 5′-amino modified aptamer DNA specific for one epitope on the target analyte plus 10 mM BS³ [Bis(sulfosuccinimidyl) suberate] or other appropriate amine-reactive bifunctional linker such as EDC [1-Ethyl-3-(3-dimethylaminopropyl)carbodiimide hydrochloride], Sulfo-EGS [Ethylene glycol bis(sulfosuccinimidylsuccinate)], Sulfo-SMCC [Sulfosuccinimidyl 4-[N-maleimidomethyl]-cyclohexane-1-carboxylate], glutaraldehyde, etc. plus 8 μM Qdot 655 ITK reagent (Invitrogen Corp.). These components are mixed in a 1 ml volume of 1× binding buffer (“1XBB”; 0.5 M NaCl, 10 mM Tris-HCl, 1 mM MgCl₂, pH 7.5-7.6) for 30 mM at room temperature (“RT”). This aptamer-QD component is purified through Sephadex G-25 or another suitable size exclusion chromatography matrix. 2) One-hundred μg of a second 5′-amine-modified DNA aptamer with specificity to a second epitope on the target analyte plus 10 μl of tosyl-activated MBs (approximately 1×10⁶ MBs, 1 to 5 microns in diameter). This aptamer-MB component is incubated at 37° C. for 2 or more hours and then collected with a strong magnet and washed 3 times in 1XBB. These two major components (aptamer-QD and aptamer-MB conjugates) are added to a polystyrene or other plastic cuvette with the addition of 1XBB up to a total volume of 2 ml. The cuvette is then lyophilized, back flushed with nitrogen gas and capped for long-term storage.

The invention has been described above in a typical embodiment and amounts of the assay components for food safety testing for low numbers of pathogenic bacteria. However, broad ranges of detection are required for other types of analytes. Therefore, considering aptamer affinity ranges and ranges of detectable fluorescence, the one-step cuvette assays may be described based on the following ratios of ranges for the two major assay components:
1) Aptamer-QD reagents: 0.5-50 nanoMoles of 5′-amono-DNA aptamer (or 10-1,000 μg of 60-100 base DNA in general) plus 10-20 miliMoles of bifunctional linker (BS³ etc., linkers are in excess) plus 0.8-80 μMoles of QDs.
2) Aptamer-MB reagents: 0.5-50 nanoMoles of 5′-amino-DNA per 10⁵-10⁷ tosyl-MBs or other appropriately derived MBs for DNA conjugation.

In general, affinities for antibodies and aptamers, 10-fold ranges for each assay component (i.e., 10-fold lower and higher) are anticipated by the current invention. The amounts of the assay components are intended to be varied, because the present invention envisions assays of varying sensitivity. Thus, the same basic assay can have assay component amounts modified to allow for situations wherein extreme sensitivity is required, and others situations wherein less sensitivity is acceptable for the application.

Prior to use, the one-step cuvette assay is reconstituted with a bulk solution which is to be tested for the presence of the desired target analyte. The bulk solution, which is in an amount anticipated to be approximately 2 ml, can be any number of various sample fluid matrices possibly containing target analytes including, but not limited to: natural waters, buffer, or diluted or undiluted food samples (e.g., milk, yoghurt, cheeses prior to solidification, meat juices, fruit juices, eggs, rinse waters from fruit and vegetable surfaces, diluted peanut butter, etc.), diluted whole blood, serum, urine, sputum or other body fluid samples.

Along with the bulk solution, an aptamer-magnetic bead conjugate (“aptamer-MB”), and an aptamer-fluorophore conjugate are added, or can be lyophilized together in situ (in a cuvette) prior to adding the target analyte. The aptamer conjugates are chosen based upon the aptamer-MB being able to bind with the target analyte at a first binding site on the target analyte, and the aptamer-fluorophore conjugate being able to bind with the target analyte at a second binding site on the target analyte. Thus, if the target analyte is present in the bulk solution, both the aptamer-MB and an aptamer-fluorophore conjugates bind with the target analyte to form an analyte-aptamer-fluorophore complex. It is also necessary that the aptamer-MBs will not bind, base pair, or hybridize with the aptamer-fluorophores in the bulk solution. If they were to attach to each other in some way, in competition with the target analyte, then the assay would produce false positives because the MB would pull the aptamer-MB-fluorophore (without a target analyte) over to the cuvette translucent surface area to be assayed.

The cuvette is recapped, shaken and mixed periodically over a 15-20 minute period, allowing the aptamer-MBs to bind with target analytes at the first binding site and the aptamer-fluorophore conjugates to bind with the target analyte at the second binding site to form an analyte-aptamer-fluorophore complex. Then the cuvette is added to a rack or other device with an external magnet set at the appropriate height to cause the analyte-aptamer-fluorophore complexes to adhere to the cuvette translucent surface area by applying an external magnetic field to attract the magnetic beads. Attracted by the magnetic field, the magnetic bead pulls the remainder of the analyte-aptamer-fluorophore complex which collects any captured analytes in a band (rectangular or square) or circular pattern at the level of a fluorometer's light path. The MBs with captured assay and target analytes are collected for 5 or more minutes and then the external magnet is removed, leaving adherent fluorescent MBs, assay and target analyte components adhering on the inner surface of the plastic cuvette as shown in FIG. 1. It is this partitioning and concentrating of the assay components and captured analytes to a thin adherent film on the inner face of the cuvette which enables discrimination of the intense assay fluorescence from the much weaker fluorescence of the bulk solution behind the adherent material. Thus, the analyte-aptamer-fluorophore complexes are effectively partitioned away from the remaining bulk solution to enhance detectability. This partitioning provides for a one-step, homogenous assay with high signal-to-noise ratio when the adherent assay is placed in a fluorometer and quantified with the appropriate excitation and emission wavelengths.

Although described above as a cuvette, the present invention is effective in any number of container or vessel geometries. Thus, the method of the present invention may be run in a tube, vial, dish, flow cell, cassette, cartridge, microfluidic chip, and any other similar type of containers. And, the container can be composed of a plethora of materials, in any shape and of any type as long as a planar area of assay material attachment in a viewing “window” is provided and nucleic acid aptamers can adhere to the material. Therefore, the assay format may also be applied to a flattened plastic or glass cassette or cartridge in which assay components might be magnetically pulled along a channel or path by an external magnet. Upon reaching a clear plastic or glass detection window the assay components would be allowed to reside in the detection window where they could adhere to the window's surface and be concentrated away from the bulk solution by the external magnet. Hence, several embodiments or geometries for the assay vessel are envisioned so long as the cuvette has a translucent surface area so as to enable a fluorescent assay. For example, the cuvette translucent surface area, on which said analyte-aptamer-fluorophore complex adheres, may be formed as a square, rectangular, round, oval, or flat container, vial, tube, cylinder, cassette, or cartridge.

It is anticipated that the cuvette may be made from polystyrene, clear plastic, or glass. But in addition, the chemistry of DNA attachment to the glass or plastic is not restricted to natural glass or simple polystyrene. Rather, logical derivative plastics and coatings (e.g., silanes, etc.) that include alkenes for electrophilic addition of DNA and hydrophobic coatings that may encourage weak force (van der Waals or dipole-dipole) interactions and adherence of DNA to the coated glass or plastic are also envisioned.

### BRIEF DESCRIPTION OF THE DRAWINGS

FIG. 1. is a schematic illustration of how the one-step adherent sandwich assay forms and is drawn to the inner face of a plastic or glass cuvette by an external magnet.

FIG. 2. shows line graphs plotting relative fluorescence intensity against the concentration of Campylobacter jejuni (C. jejuni) bacteria.

FIG. 3. shows a series of fluorescence emission spectra related to detection of serial ten-fold dilutions of Campylobacter jejuni bacteria in neat buffer (1XBB) and various diluted food matrices as indicated in the figure. Excitation was at 380 nm with a photomultiplier tube setting of 900 Volts.

FIG. 4 illustrates a typical one-step assay capable of detecting 10 live C. jejuni bacteria in undiluted chicken “juice” (serous fluid collected from chicken legs prior to cooking). Data points represent the means and standard deviations of five independent readings (N=5).

### DETAILED DESCRIPTION OF THE PREFERRED EMBODIMENT

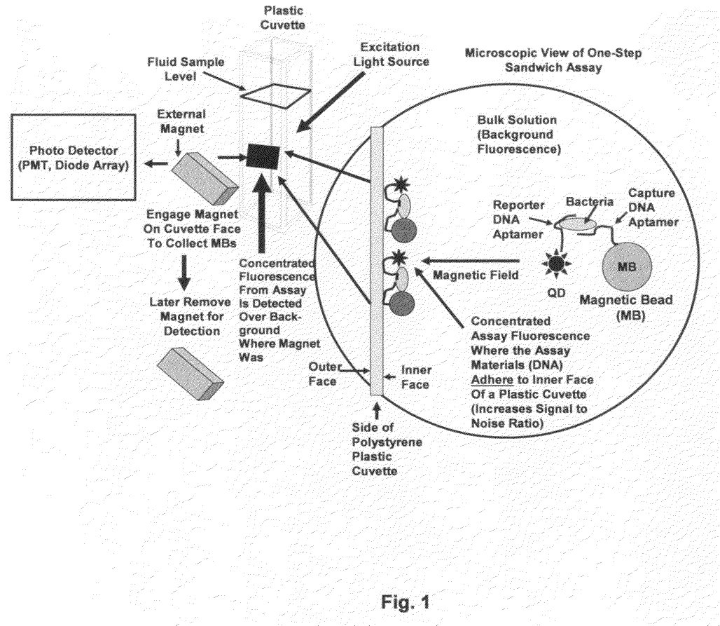

Referring to the figures, FIG. 1. provides a schematic representation of the one-step adherent sandwich assay concept. In this concept, a DNA or possibly an RNA aptamer has been conjugated to a magnetic bead and used to capture a target analyte (bacterial cell in this example). Capture is achieved by specific aptamer binding to an epitope on the bacterial surface. Likewise, another epitope is bound by an aptamer-quantum dot conjugate or other aptamer-fluorophore reporter reagent simultaneously for fluorescent detection. Because the sandwich assay contains DNA or RNA, it is subject to adhering to some forms of charged glass or charged or uncharged plastics such as polystyrene and its derivatives by electrostatic and/or other weak forces such as dipole-dipole or Van der Waals interactions and possibly covalent electrophilic addition to alkenes or the styrene rings (Bensimon et al., 1994). Adherence is promoted by the addition of an external attractive magnetic force such as a strong Cobalt, Neodynium, or other rare earth magnet. After the external magnet is disengaged, the assay materials still adhere to the inner face of the cuvette due to interaction of DNA with the polystyrene or other plastic or glass materials. This adherence partitions the assay along with captured and labeled bacteria or other analytes from the bulk solution. If the solution is illuminated from the opposite side by an excitation source and the cuvette face with adherent assay materials is placed proximal to a photodetector, rapid, sensitive, one-step detection is enabled. Once adherence of all the aptamer-MB-bacteria-aptamer-QD complexes occurs on the surface, the adherent material emits a much brighter fluorescent signal than the bulk solution which contains free aptamer-QD or aptamer-fluorophore conjugates.

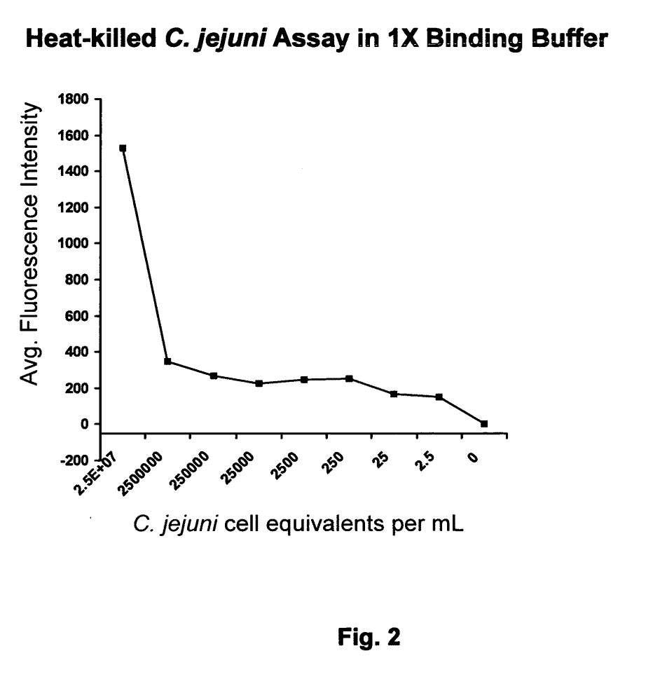

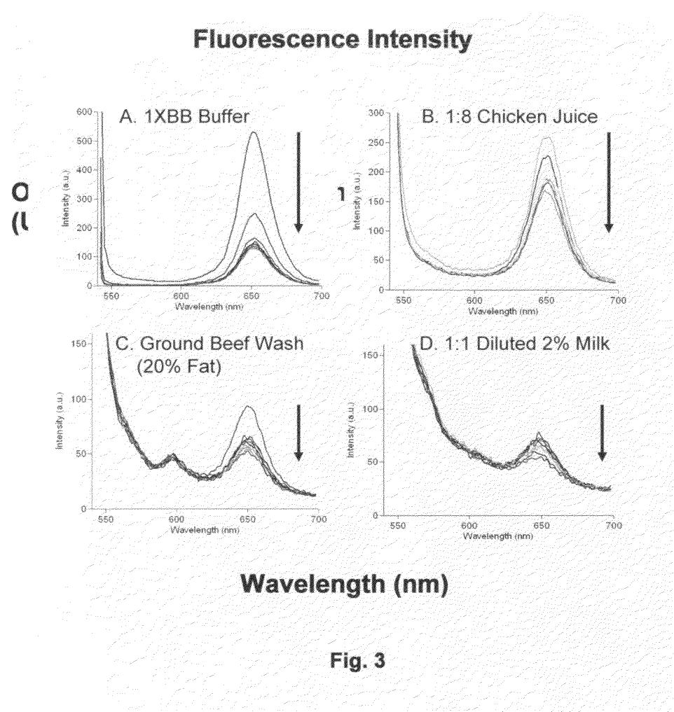

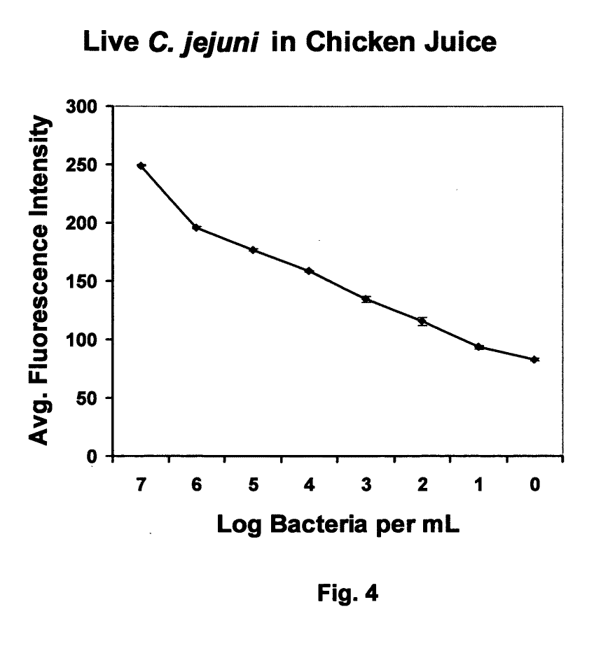

FIG. 2. shows line graphs plotting relative fluorescence intensity against the concentration of Campylobacter jejuni (C. jejuni) bacteria detected in neat buffer (1× binding buffer; 1XBB) down to a level of approximately 2 bacterial cells per milliliter using the one-step adherent DNA aptamer-MB-aptamer red QD (Q-dot 655 nm) sandwich assay without a wash step. Five independent readings were taken per data point with the green (Rhodamine) channel of a Turner Biosystems, Inc. handheld fluorometer. Error bars which are not visible due to their small numerical values represent the standard deviations of the 5 readings. The preferred embodiment for the adherent one-step washless aptamer-MB/aptamer-QD or aptamer-fluorophore assays is in a plastic polystyrene cuvette using lyophilized (freeze-dried) sandwich assay materials with long shelf-life that are rehydrated as needed. Their fluorescence can be assessed after a 15-20 minute capture and 5 minute magnetic collection period via a table top spectrofluorometer, or portable fluorometers such as the Turner Biosystem's Picofluor™ or Invitrogen's Q-Bit™ or other such fluorescence reader devices. The primarily linear photoresponse to logarithmic changes in bacterial concentration seen in FIGS. 2 and 4 is probably attributable to a photodiode detector in the fluorometer, such as is found in the Picofluor™, versus the more sensitive and exponentially responsive photomultiplier tube (PMT) used for data collection by a spectrofluorometer in FIG. 3.

FIG. 3. shows a series of fluorescence emission spectra from ten-fold serial dilutions of 25 million heat-killed C. jejuni bacteria per ml (highest peak) to 2.5 bacteria per ml and then zero bacteria per ml (lowest peak) detected by use of a Cary-Varian spectrofluorometer and the one-step plastic-adherent aptamer-MB/aptamer-red QD (Q-dot 655 nm) sandwich assay without a wash step directly in various food matrices as indicated. The arrows indicate the direction of increasing 2-fold dilutions or decreasing bacterial concentration. The assays are generally described herein as using a fluorescence intensity reporter method, which is a simple measure of fluorescence brightness, for detecting and quantifying the analyte-aptamer conjugate. Alternatively, the fluorescence intensity reporter method may be substituted by time-resolved fluorescence, chemiluminescence, electrical detection, electrochemical detection, electrochemiluminescence, phosphorescence, or radioisotopic detection instead of simple fluorescence intensity-based detection.

FIG. 4. illustrates a typical one-step assay capable of detecting 10 live C. jejuni bacteria in chicken juice (collected blood and fat globules from a fresh grocery store chicken product). Five independent readings were taken per data point with the green (Rhodamine) channel of a Turner Biosystems, Inc. handheld Picofluor™ fluorometer. Error bars which are barely visible due to their small numerical values represent the standard deviations of the 5 readings.

No wash steps are required and detection can be achieved directly in various food, environmental, or body fluid matrices as illustrated in FIGS. 3 and 4.

### Example 1

### One-Step, Washless Ultra sensitive Detection of Campylobacter jejuni in Various Food Matrices

The invention has been used to detect as few as 2 live or dead C. jejuni bacterial cells (a common foodborne pathogen) in neat buffer and various food matrices as shown in FIGS. 2-4. In these assays, two different C. jejuni Sequences (designated C2 and C3 or SEQ ID NOs 2 and 3) were 5′-amine modified during solid-phase DNA synthesis and attached to either 1,000 tosyl-M280 (2.8 micron diameter) Dynal (Invitrogen, Inc.) MBs or 0.24 picoliters of Q-dot 655 ITK reagent (Invitrogen, Inc.) per test. The C2 aptamer (SEQ ID NO. 2) was used for capture on the surface of tosyl-MBs and the C3 aptamer (SEQ ID NO. 3) was used as the reporter reagent after attachment to the Q-dot 655 ITK reagent via BS³ (bis-suberate bifunctional linker from Pierce Chemical Co.). The reagents were purified, mixed together and lyophilized in plastic cuvettes. The powdered assays were later backflushed with nitrogen and capped. Upon rehydration, the adherent one-step sandwich assays were used to detect live or dead (heat-killed) C. jejuni cells with the very sensitive results depicted in FIGS. 2-4. Other aptamers chosen for capture and reporter functions from SEQ ID NOs. 1-6 can be substituted in Campylobacter assays which are functional, but result in somewhat less sensitive detection.

### Example 2

### One-Step, Washless Ultrasensitive Detection of Escherichia coli O157:H7 and other toxigenic (Shiga or Verotoxin-producing) E. coli in Various Food Matrices

The present invention has potential to be used for detection of enterohemorraghic E. coli O157:H7 in and on various foods via binding of aptamers to the outer saccharides of 0157 lipopolysaccharide (LPS) and the H7 flagellar antigen. Aptamer sequences from SEQ ID NOs. 7-20 could be chosen for capture (aptamer-MB conjugate) or reporter (aptamer-fluorophore conjugate) functions and used to detect E. coli O157:H7 in or on foods. Alternatively, outer membrane proteins (OMPs) common to many species of E. coli can be used for aptamer-MB-based capture (or identification) of the E. coli bacterial cells followed by specific identification of the E. coli strain or serotype using LPS-specific aptamer-QD reporter reagents to complete the sandwich assay. Aptamer SEQ ID NOs. 279-322 can be used for E. coli OMP recognition and capture. In yet another embodiment non-O157:H7 toxigenic E. coli bacteria can be sensitively identified by their secretion of Shiga or Verotoxins (types 1 and 2 or Stx-1 and Stx-2). Many other strains of E. coli including O126 can produce deadly disease in humans and the common thread among these lethal pathogens is the secretion of Stx. Therefore, a very useful embodiment of the invention would be detection of Stx-1 and/or Stx-2 using any of the DNA aptamer sequences identified by SEQ ID NOs. 323-352.

### Example 3

### One-Step, Washless Ultrasensitive Detection of Listeria monocytogenes in Various Food Matrices

The present invention has potential to be used for detection of lethal L. monocytogenes in and on various foods via binding to the listerolysin (LO) surface protein. Aptamer sequences from SEQ ID NOs. 21-52 could be chosen for capture (aptamer-MB conjugate) or reporter (aptamer-fluorophore conjugate) functions and used to detect LO and L. moncytogenes in or on foods.

### Example 4

### One-Step, Washless Ultrasensitive Detection of Salmonella typhimurium in Various Food Matrices

The present invention has potential to be used for detection of S. typhimurium and other Salmonella species (S. typhi etc.) in and on various foods. S. typhimurium has been renamed Salmonella enterica serovar Typhimurium, but many microbiologists and lay people still refer to the microbe as S. typhimurium. Aptamer sequences from SEQ ID NOs. 53-68 could be chosen for capture (aptamer-MB conjugate) or reporter (aptamer-fluorophore conjugate) functions for detection of Salmonella typhimurium LPS bacteria in or on foods. In addition, aptamer SEQ ID NOs. 353-392 could be used for capture or identification of S. typhimurium OMPs. These S. typhimurium DNA aptamer sequences are unique and bear no resemblance to those recently reported by Joshi et al. (2008).

### Example 5

### Detection of Fecal Contamination of Water Supplies

The present invention has the potential to detect all species of Escherichia coli bacteria in recreational, treated waste water, and drinking water supplies using aptamer DNA SEQ ID NOs. 69-122 directed against common core components of LPS for capture and reporter functions. The present invention has the potential to detect all species of Enterococcus bacteria (another common fecal indicator organism) in recreational, treated waste water, and drinking water supplies using aptamer DNA SEQ ID NOs. 123-130 directed against common teichoic acid moieties for capture and reporter functions.

### Example 6

### Detection of Leishmania Parasites in Skin Lesions or Visceral Fluids

The present invention has the potential to detect Leishmania donovani or L. tropica parasites in skin lesions of body fluids and other samples using aptamer DNA Sequences chosen from SEQ ID NOs. 131-134 directed against surface proteins of common to both Leishmania species for capture and reporter functions.

### Example 7

### Detection of Encapsultaed Vegetative Bacillus anthracis Bacteria in Blood and Body Fluids

The invention has the potential to detect encapsulated B. anthracis (anthrax) vegetative bacteria in blood and body fluids and other samples using aptamer DNA Sequences chosen from SEQ ID NOs. 135-138 directed against surface poly-D-glutamic acid (PDGA) capsular materials for capture and reporter functions.

### Example 8

### Detection of Small Molecules (<1,000 Daltons) in Environmental and Clinical Samples

The invention has the potential to detect small molecules of <1,000 Daltons, if the target has two distinct and accessible epitopes for attachment of capture and reported aptamers to enable a sandwich assay format. Among such small molecule targets would be organophosphorus pesticides (such as diazinon and malathion) in environmental water, soil, or mud samples as well as blood and body fluids and other samples using aptamer DNA Sequences chosen from SEQ ID NOs. 139-154 directed against different ends of the pesticide molecule for capture and reporter functions. In addition, vitamins such as 25-hydroxyvitamin D₃ (calcidiol; SEQ ID NOs. 243-274), the neurotransmitter acetylcholine (ACh; SEQ ID Nos. 393-416) might be viable targets for this novel adherent assay format

### Example 9

### Detection of Foot-and-Mouth Disease (FMD) and Related Viruses

The invention has the potential to detect FMD and related viruses in blood and body fluids and other samples using aptamer DNA Sequences chosen from SEQ ID NOs. 155-164 directed against a conserved 16-amino acid peptide from several 0 serotypes of FMD for capture and reporter functions.

### Example 10

### Detection of Bone Resorption or Synthesis Markers in Blood and Body Fluids

The invention has the potential to detect markers of bone loss such as cathepsin K, C-terminal telopeptides (CTx) and N-terminal telopeptides (NTx) of collagen, hydroxylysine (HL), osteocalcin fragments (OCF), etc. due to the effects of low gravity during lengthy spaceflights or osteoporosis and aging in blood, urine and other body fluids and other samples using aptamer DNA Sequences chosen from SEQ ID NOs. 165-242 directed against unique epitopes on each type of bone marker. The invention also has the potential to detect and discriminate various isomers of vitamin D associated with bone formation chosen from SEQ ID NOs. 243-274 for capture and reporter functions.

### Example 11

### Detection of Botulinum Toxin A and Related Biotoxins

The invention has the potential to detect Clostridum botulinum toxins which affect humans and animals (serotypes A-F) and related bacterial, harmful algal bloom (HAB, dinoflagellate), marine (shellfish-related), or plant toxins such as tetanus toxin, cholera and diphtheria toxins, shiga and verotoxins, staphylococcal enterotoxins, cyanotoxins, azaspiracids, brevetoxins, ciguatoxins, gonyautotoxins, domoic acid isomers, maitotoxins, palytoxins, yessotoxins, saxitoxins, ricin, gelonin, abrin, spider and snake venoms, etc. in blood and body fluids and other samples using aptamer DNA sequences in the adherent sandwich format. Aptamer sequences chosen from SEQ ID NOs. 275-278 in particular can be used to for detection of botulinum type A light chains or the holotoxin.

### Example 12

### Detection of Quorum Sensing Molecules in Bacterial Infections

Many species of bacteria are now known to communicate chemically via secreted small molecules. Many Gram negative bacterial pathogens commonly use a family of small molecules called acylhomoserine lactones (AHLs) to communicate between bacterial cells to sense when a critical concentration of cells or “quorum” has been reached to enable effective infection of a host organism. AHLs control induction of pathogenesis and virulence factors such as expression of adherence proteins and toxins. Therefore, early sensing of AHLs could indicate an imminent Gram negative bacterial infection and prompt a physician to administer the appropriate antibiotics to prevent an infection or more severe sepsis. AHLs do commonly possess two different ends or potential epitopes and are therefore potential candidates for the one-step plastic-adherent DNA aptamer-MB-aptamer-QD or other aptamer-reporter sandwich assays described herein. Sequence ID Nos. 417-426 illustrate potential aptamer DNA sequences developed against and reactive with the family of Gram negative bacterial AHLs for diagnostics.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention. Such alternative embodiments may include, but are not limited to changes in the reporter method including chemiluminescence, electrical detection, electrochemical detection, electrochemiluminescence, phosphorescence, and radioisotopic detection instead of fluorescence-based detection.

TABLE 1
DNA Aptamer Sequence ID Nos.
Campylobacter jejuni Surface Protein Aptamers
SEQ ID NO. 1
(C1)
CATCCGTCACACCTGCTCTGGGGAGGGTGGCGCCCGTCTCGGTGGTGTTG
GCTCCCGTATCA
SEQ ID NO. 2
(C2)
CATCCGTCACACCTGCTCTGGGATAGGGTCTCGTGCTAGATGTGGTGTTG
GCTCCCGTATCA
SEQ ID NO. 3
(C3)
CATCCGTCACACCTGCTCTGGACCGGCGCTTATTCCTGCTTGTGGTGTTG
GCTCCCGTATCA
SEQ ID NO. 4
(C4)
CATCCGTCACACCTGCYCTGGAGCTGATATTGGATGGTCCGGTGGTGTTG
GCTCCCGTATCA
SEQ ID NO. 5
(C5)
CATCCGTCACACCTGCYCYGCCCAGAGCAGGTGTGACGGATGTGGTGTTG
GCTCCCGTATCA
SEQ ID NO. 6
(C6)
CATCCGTCACACCTGCYCYGCCGGACCATCCAATATCAGCTGTGGTGTTG
GCTCCCGTATCA
E. coli O157 Lipopolysaccharide (LPS) Aptamers
SEQ ID NO. 7
(E-5F)
ATCCGTCACACCTGCTCTGGTGGAATGGACTAAGCTAGCTAGCGTTTTAA
AAGGTGGTGTTGGCTCCCGTAT
SEQ ID NO. 8
(E-11F)
ATCCGTCACACCTGCTCTGTAAGGGGGGGGAATCGCTTTCGTCTTAAGAT
GACATGGTGTTGGCTCCCGTAT
SEQ ID NO.9
(E-12F)
ATCCGTCACACCTGCTCTGCCGGACCATCCAATATCAGCTGTGGTGTTGG
CTCCCGTAT
SEQ ID NO. 10
(E-16F)
ATCCGTCACACCTGCTCTATCCGTCACGCCTGCTCTATCCGTCACACCTG
CTCTGGTGTTGGCTCCCGTAT
SEQ ID NO. 11
(E-17F)
ATCCGTCACACCTGCTCTATCAAATGTGCAGATATCAAGACGATTTGTAC
AAGATGGTGTTGGCTCCCGTAT
SEQ ID NO. 12
(E-18F)
ATCCGTCACACCTGCTCTGTAGATGGCAAGGCATAAGCGTCCGGAACGAT
AGAATGGTGTTGGCTCCCGTAT
SEQ ID NO. 13
(E-19F)
ATCCGTCACACCTGCTCTGTAGATGGCAAGGCATAAGCGTCCGGAACGAT
AGAATGGTGTTGGCTCCCGTAT
SEQ ID NO. 14
(E-5R)
ATACGGGAGCCAACACCACCTTTTAAAACGCTAGCTAGCTTAGTCCATTC
CACCAGAGCAGGTGTGACGGAT
SEQ ID NO. 15
(E-11R)
ATACGGGAGCCAACACCATGTCATCTTAAGACGAAAGCGATTCCCCCCCC
TTACAGAGCAGGTGTGACGGAT
SEQ ID NO. 16
(E-12R)
ATACGGGAGCCAACACCACAGCTGATATTGGATGGTCCGGCAGAGCAGGT
GTGACGGAT
SEQ ID NO. 17
(E-16R)
ATACGGGAGCCAACACCAGAGCAGGTGTGACGGATAGAGCAGGCGTGACG
GATAGAGCAGGTGTGACGGAT
SEQ ID NO. 18
(E-17R)
ATACGGGAGCCAACACCATCTTGTACAAATCGTCTTGATATCTGCACATT
TGATAGAGCAGGTGTGACGGAT
SEQ ID NO. 19
(E-18R)
ATACGGGAGCCAACACCATTCTATCGTTCCGGACGCTTATGCCTTGCCAT
CTACAGAGCAGGTGTGACGGAT
SEQ ID NO. 20
(E-19R)
ATACGGGAGCCAACACCATTCTATCGTTCCGGACGCTTATGCCTTGCCAT
CTACAGAGCAGGTGTGACGGAT
Listeriolysin (A surface protein on Listeria
monocytogenes) Aptamers
SEQ ID NO. 21
(LO-10F)
ATCCGTCACACCTGCTCTGCCGGACCATCCAATATCAGCTGTGGTGTTGG
CTCCCGTAT
SEQ ID NO. 22
(LO-11F)
ATCCGTCACACCTGCTCTGGTGGAATGGACTAAGCTAGCTAGCGTTTTAA
AAGGTGGTGTTGGCTCCCGTAT
SEQ ID NO. 23
(LO-13F)
ATCCGTCACACCTGCTCTTAAAGTAGAGGCTGTTCTCCAGACGTCGCAGG
AGGATGGTGTTGGCTCCCGTAT
SEQ ID NO. 24
(LO-15F)
ATCCGTCACACCTGCTCTGTAGATGGCAAGGCATAAGCGTCCGGAACGAT
AGAATGGTGTTGGCTCCCGTAT
SEQ ID NO. 25
(LO-16F)
ATCCGTCACACCTGCTCTGTAGATGGCAAGGCATAAGCGTCCGGAACGAT
AGAATGGTGTTGGCTCCCGTAT
SEQ ID NO. 26
(LO-17F)
ATACGGGAGCCAACACCA
CAGCTGATATTGGATGGTCCGGCAGAGCAGGTGTGACGGAT
SEQ ID NO. 27
(LO-19F)
ATCCGTCACACCTGCTCTTGGGCAGGAGCGAGAGACTCTAATGGTAAGCA
AGAATGGTGTTGGCTCCCGTAT
SEQ ID NO. 28
(LO-20F)
ATCCGTCACACCTGCTCTCCAACAAGGCGACCGACCGCATGCAGATAGCC
AGGTTGGTGTTGGCTCCCGTAT
SEQ ID NO. 29
(LO-10R)
ATACGGGAGCCAACACCACAGCTGATATTGGATGGTCCGGCAGAGCAGGT
GTGACGGAT
SEQ ID NO. 30
(LO-11R)
ATACGGGAGCCAACACCACCTTTTAAAACGCTAGCTAGCTTAGTCCATTC
CACCAGAGCAGGTGTGACGGAT
SEQ ID NO. 31
(LO-13R)
ATACGGGAGCCAACACCATCCTCCTGCGACGTCTGGAGAACAGCCTCTAC
TTTAAGAGCAGGTGTGACGGAT
SEQ ID NO. 32
(LO-15R)
ATACGGGAGCCAACACCATTCTATCGTTCCGGACGCTTATGCCTTGCCAT
CTACAGAGCAGGTGTGACGGAT
SEQ ID NO. 33
(LO-16R)
ATACGGGAGCCAACACCATTCTATCGTTCCGGACGCTTATGCCTTGCCAT
CTACAGAGCAGGTGTGACGGAT
SEQ ID NO. 34
(LO-17R)
ATCCGTCACACCTGCTCTGCCGGACCATCCAATATCAGCTGTGGTGTTGG
CTCCCGTAT
SEQ ID NO. 35
(LO-19R)
ATACGGGAGCCAACACCATTCTTGCTTACCATTAGAGTCTCTCGCTCCTG
CCCAAGAGCAGGTGTGACGGAT
SEQ ID NO. 36
(LO-20R)
ATACGGGAGCCAACACCAACCTGGCTATCTGCATGCGGTCGGTCGCCTTG
TTGGAGAGCAGGTGTGACGGAT
Listeriolysin (Alternate form of Listeria surface
protein designated “Pest-Free”) Aptamers
SEQ ID NO. 37
(LP-3F)
ATCCGTCACACCTGCTCTGTAGATGGCAAGGCATAAGCGTCCGGAACGAT
AGAATGGTGTTGGCTCCCGTAT
SEQ ID NO. 38
(LP-11F)
ATCCGTCACACCTGCTCTAACCAAAAGGGTAGGAGACCAAGCTAGCGATT
TGGATGGTGTTGGCTCCCGTAT
SEQ ID NO. 39
(LP-13F)
ATCCGTCACACCTGCTCTGCCGGACCATCCAATATCAGCTGTGGTGTTGG
CTCCCGTAT
SEQ ID NO. 40
(LP-14F)
ATCCGTCACACCTGCTCTGAAGCCTAACGGAGAAGATGGCCCTACTGCCG
TAGGTGGTGTTGGCTCCCGTAT
SEQ ID NO. 41
(LP-15F)
ATCCGTCACACCTGCTCTACTAAACAAGGGCAAACTGTAAACACAGTAGG
GGCGTGGTGTTGGCTCCCGTAT
SEQ ID NO. 42
(LP-17F)
ATCCGTCACACCTGCTCTGGTGTTGGCTCCCGTATAGCTTGGCTCCCGTA
TGGTGTTGGCTCCCGTAT
SEQ ID NO. 43
(LP-18F)
ATCCGTCACACCTGCTCTGTCGCGATGATGAGCAGCAGCGCAGGAGGGAG
GGGGTGGTGTTGGCTCCCGTAT
SEQ ID NO. 44
(LP-20F)
ATCCGTCACACCTGCTCTGATCAGGGAAGACGCCAACACTGGTGTTGGCT
CCCGTAT
SEQ ID NO. 45
(LP-3R)
ATACGGGAGCCAACACCATTCTATCGTTCCGGACGCTTATGCCTTGCCAT
CTACAGAGCAGGTGTGACGGAT
SEQ ID NO. 46
(LP-11R)
ATACGGGAGCCAACACCATCCAAATCGCTAGCTTGGTCTCCTACCCTTTT
GGTTAGAGCAGGTGTGACGGAT
SEQ ID NO. 47
(LP-13R)
ATACGGGAGCCAACACCACAGCTGATATTGGATGGTCCGGCAGAGCAGGT
GTGACGGAT
SEQ ID NO. 48
(LP-14R)
ATACGGGAGCCAACACCACCTACGGCAGTAGGGCCATCTTCTCCGTTAGG
CTTCAGAGCAGGTGTGACGGAT
SEQ ID NO. 49
(LP-15R)
ATACGGGAGCCAACACCACGCCCCTACTGTGTTTACAGTTTGCCCTTGTT
TAGTAGAGCAGGTGTGACGGAT
SEQ ID NO. 50
(LP-17R)
ATACGGGAGCCAACACCATACGGGAGCCAAGCTATACGGGAGCCAACACC
AGAGCAGGTGTGACGGAT
SEQ ID NO. 51
(LP-18R)
ATACGGGAGCCAACACCACCCCCTCCCTCCTGCGCTGCTGCTCATCATCG
CGACAGAGCAGGTGTGACGGAT
SEQ ID NO. 52
(LP-20R)
ATACGGGAGCCAACACCAGTGTTGGCGTCTTCCCTGATCAGAGCAGGTGT
GACGGAT
Salmonella typhimurium lipopolysaccharide(LPS)
Aptamers
SEQ ID NO. 53
(St-7F)
ATCCGTCACACCTGCTCTGTCCAAAGGCTACGCGTTAACGTGGTGTTGGC
TCCCGTAT
SEQ ID NO. 54
(St-10F)
ATCCGTCACACCTGCTCTGGAGCAATATGGTGGAGAAACGTGGTGTTGGC
TCCCGTAT
SEQ ID NO. 55
(St-11F)
ATCCGTCACACCTGCTCTGCCGGACCATCCAATATCAGCTGTGGTGTTGG
CTCCCGTAT
SEQ ID NO. 56
(St-15F)
ATCCGTCACACCTGCTCTGAACAGGATAGGGATTAGCGAGTCAACTAAGC
AGCATGGTGTTGGCTCCCGTAT
SEQ ID NO. 57
(St-16F)
ATCCGTCACACCTGCTCTGGCGGACAGGAAATAAGAATGAACGCAAAATT
TATCTGGTGTTGGCTCCCGTAT
SEQ ID NO. 58
(St-18F)
ATCCGTCACACCTGCTCTACGCAACGCGACAGGAACATTCATTATAGAAT
GTGTTGGTGTTGGCTCCCGTAT
SEQ ID NO. 59
(St-19F)
ATCCGTCACACCTGCTCTCGGCTGCAATGCGGGAGAGTAGGGGGGAACCA
AACCTGGTGTTGGCTCCCGTAT
SEQ ID NO. 60
(St-20F)
ATCCGTCACACCTGCTCTATGACTGGAACACGGGTATCGATGATTAGATG
TCCTTGGTGTTGGCTCCCGTAT
SEQ ID NO. 61
(St-7R)
ATACGGGAGCCAACACCACGTTAACGCGTAGCCTTTGGACAGAGCAGGTG
TGACGGAT
SEQ ID NO. 62
(St-10R)
ATACGGGAGCCAACACCACGTTTCTCCACCATATTGCTCCAGAGCAGGTG
TGACGGAT
SEQ ID NO. 63
(St-11R)
ATACGGGAGCCAACACCACAGCTGATATTGGATGGTCCGGCAGAGCAGGT
GTGACGGAT
SEQ ID NO. 64
(St-15R)
ATACGGGAGCCAACACCATGCTGCTTAGTTGACTCGCTAATCCCTATCCT
GTTCAGAGCAGGTGTGACGGAT
SEQ ID NO. 65
(St-16R)
ATACGGGAGCCAACACCAGATAAATTTTGCGTTCATTCTTATTTCCTGTC
CGCCAGAGCAGGTGTGACGGAT
SEQ ID NO. 66
(St-18R)
ATACGGGAGCCAACACCAACACATTCTATAATGAATGTTCCTGTCGCGTT
GCGTAGAGCAGGTGTGACGGAT
SEQ ID NO. 67
(St-19R)
ATACGGGAGCCAACACCAGGTTTGGTTCCCCCCTACTCTCCCGCATTGCA
GCCGAGAGCAGGTGTGACGGAT
SEQ ID NO. 68
(St-20R)
ATACGGGAGCCAACACCAAGGACATCTAATCATCGATACCCGTGTTCCAG
TCATAGAGCAGGTGTGACGGAT
Fecal Contamination Indicator (Core LPS Antigens)
Aptamers
SEQ ID NO. 69
(Glucosamine(G)1F)
ATCCGTCACACCTGCTCTAATTAGGATACGGGGCAACAGAACGAGAG
GGGGGAATGGTGTTGGCTCCCGTAT
SEQ ID NO. 70
(G2F)
ATCCGTCACACCTGCTCTCGGACCAGGTCAGACAAGCACATCGGATAT
CCGGCTGGTGTTGGCTCCCGTAT
SEQ ID NO. 71
(G5F)
ATCCGTCACACCTGCTCTTGAGTCAAAGAGTTTAGGGAGGAGCTAACA
TAACAGTGGTGTTGGCTCCCGTAT
SEQ ID NO. 72
(G7F)
ATCCGTCACACCTGCTCTAACAACAATGCATCAGCGGGCTGGGAACGC
ATGCGGTGGTGTTGGCTCCCGTAT
SEQ ID NO. 73
(G8F)
ATCCGTCACACCTGCTCTGAACAGGTTATAAGCAGGAGTGATAGTTTC
AGGATCTGGTGTTGGCTCCCGTAT
SEQ ID NO. 74
(G9F)
ATCCGTCACACCTGCTCTCGGCGGCTCGCAAACCGAGTGGTCAGCACC
CGGGTTGGTGTTGGCTCCCGTAT
SEQ ID NO. 75
(G10F)
ATCCGTCACACCTGCTCTGCGCAAGACGTAATCCACAAGACCGTGAAA
ACATAGTGGTGTTGGCTCCCGTAT
SEQ ID NO. 76
(G1R)
ATACGGGAGCCAACACCATTCCCCCCTCTCGTTCTGTTGCCCCGTATCC
TAATTAGAGCAGGTGTGACGGAT
SEQ ID NO. 77
(G2R)
ATACGGGAGCCAACACCAGCCGGATATCCGATGTGCTTGTCTGACCTG
GTCCGAGAGCAGGTGTGACGGAT
SEQ ID NO. 78
(G5R)
ATACGGGAGCCAACACCACTGTTATGTTAGCTCCTCCCTAAACTCTTTG
ACTCAAGAGCAGGTGTGACGGAT
SEQ ID NO. 79
(G7R)
ATACGGGAGCCAACACCACCGCATGCGTTCCCAGCCCGCTGATGCATT
GTTGTTAGAGCAGGTGTGACGGAT
SEQ ID NO. 80
(G8R)
ATACGGGAGCCAACACCAGATCCTGAAACTATCACTCCTGCTTATAAC
CTGTTCAGAGCAGGTGTGACGGAT
SEQ ID NO. 81
(G9R)
ATACGGGAGCCAACACCAACCCGGGTGCTGACCACTCGGTTTGCGAG
CCGCCGAGAGCAGGTGTGACGGAT
SEQ ID NO. 82
(G10R)
ATACGGGAGCCAACACCACTATGTTTTCACGGTCTTGTGGATTACGTC
TTGCGCAGAGCAGGTGTGACGGAT
SEQ ID NO. 83
(KDO (K) Antigen 2F)
ATCCGTCACACCTGCTCTAGGCGTAGTGACTAAGTCGCGCGAAAATCA
CAGCATTGGTGTTGGCTCCCGTAT
SEQ ID NO. 84
(K5F)
ATCCGTCACACCTGCTCTCAGCGGCAGCTATACAGTGAGAACGGACTA
GTGCGTTGGTGTTGGCTCCCGTAT
SEQ ID NO. 85
(K7F)
ATCCGTCACACCTGCTCTGGCAAATAATACTAGCGATGATGGATCTGG
ATAGACTGGTGTTGGCTCCCGTAT
SEQ ID NO. 86
(K8F)
ATCCGTCACACCTGCTCTGGGGGTGCGACTTAGGGTAAGTGGGAAAGA
CGATGCTGGTGTTGGCTCCCGTAT
SEQ ID NO. 87
(K9F)
ATCCGTCACACCTGCTCTCAAGAGGAGATGAACCAATCTTAGTCCGAC
AGGCGGTGGTGTTGGCTCCCGTAT
SEQ ID NO. 88
(K10F)
ATCCGTCACACCTGCTCTGGCCCGGAATTGTCATGACGTCACCTACAC
CTCCTGTGGTGTTGGCTCCCGTAT
SEQ ID NO. 89
(K2R)
ATACGGGAGCCAACACCAATGCTGTGATTTTCGCGCGACTTAGTCACT
ACGCCTAGAGCAGGTGTGACGGAT
SEQ ID NO. 90
(K5R)
ATACGGGAGCCAACACCAACGCACTAGTCCGTTCTCACTGTATAGCTG
CCGCTGAGAGCAGGTGTGACGGAT
SEQ ID NO. 91
(K7R)
ATACGGGAGCCAACACCAGTCTATCCAGATCCATCATCGCTAGTATTA
TTTGCCAGAGCAGGTGTGACGGAT
SEQ ID NO. 92
(K8R)
ATACGGGAGCCAACACCAGCATCGTCTTTCCCACTTACCCTAAGTCGC
ACCCCCAGAGCAGGTGTGACGGAT
SEQ ID NO. 93
(K9R)
ATACGGGAGCCAACACCACCGCCTGTCGGACTAAGATTGGTTCATCTC
CTCTTGAGAGCAGGTGTGACGGAT
SEQ ID NO. 94
(K10R)
ATACGGGAGCCAACACCACAGGAGGTGTAGGTGACGTCATGACAATT
CCGGGCCAGAGCAGGTGTGACGGAT
SEQ ID NO. 95
(Whole LPS from E. coli O111: B4(L)1F)
ATCCGTCACCCCTGCTCTCGTCGCTATGAAGTAACAAAGATAGGAGCA
ATCGGGTGGTGTTGGCTCCCGTAT
SEQ ID NO. 96
(L3F)
ATCCGTCACACCTGCTCTAACGAAGACTGAAACCAAAGCAGTGACAG
TGCTGAATGGTGTTGGCTCCCGTAT
SEQ ID NO. 97
(L4F)
ATCCGTCACACCTGCTCTCGGTGACAATAGCTCGATCAGCCCAAAGTC
GTCAGATGGTGTTGGCTCCCGTAT
SEQ ID NO. 98
(L6F)
ATCCGTCACACCTGCTCTAACGAAATAGACCACAAATCGATACTTTAT
GTTATTGGTGTTGGCTCCCGTAT (71)
SEQ ID NO. 99
(L7F)
ATCCGTCACACCTGCTCTGTCGAATGCTCTGCCTGGAAGAGTTGTTAG
CAGGGATGGTGTTGGCTCCCGTAT
SEQ ID NO. 100
(L8F)
ATCCGTCACACCTGCTCTTAAGCCGAGGGGTAAATCTAGGACAGGGGT
CCATGATGGTGTTGGCTCCCGTAT
SEQ ID NO. 101
(L9F)
ATCCGTCACACCTGCTCTACTGGCCGGCTCAGCATGACTAAGAAGGAA
GTTATGTGGTGTTGGCTCCCGTAT
SEQ ID NO. 102
(L10F)
ATCCGTCACACCTGCTCTGGTACGAATCACAGGGGATGCTGGAAGCTT
GGCTCTTGGTGTTGGCTCCCGTAT
SEQ ID NO. 103
(L1R)
ATACGGGAGCCAACACCACCCGATTGCTCCTATCTTTGTTACTTCATAG
CGACGAGAGCAGGGGTGACGGAT
SEQ ID NO. 104
(L3R)
ATACGGGAGCCAACACCATTCAGCACTGTCACTGCTTTGGTTTCAGTC
TTCGTTAGAGCAGGTGTGACGGAT
SEQ ID NO. 105
(L4R)
ATACGGGAGCCAACACCATCTGACGACTTTGGGCTGATCGAGCTATTG
TCACCGAGAGCAGGTGTGACGGAT
SEQ ID NO. 106
(L6R)
ATACGGGAGCCAACACCAATAACATAAAGTATCGATTTGTGGTCTATT
TCGTTAGAGCAGGTGTGACGGAT
SEQ ID NO. 107
(L7R)
ATACGGGAGCCAACACCATCCCTGCTAACAACTCTTCCAGGCAGAGCA
TTCGACAGAGCAGGTGTGACGGAT
SEQ ID NO. 108
(L8R)
ATACGGGAGCCAACACCATCATGGACCCCTGTCCTAGATTTACCCCTC
GGCTTAAGAGCAGGTGTGACGGAT
SEQ ID NO. 109
(L9R)
ATACGGGAGCCAACACCACATAACTTCCTTCTTAGTCATGCTGAGCCG
GCCAGTAGAGCAGGTGTGACGGAT
SEQ ID NO. 110
(L10R)
ATACGGGAGCCAACACCAAGAGCCAAGCTTCCAGCATCCCCTGTGATT
CGTACCAGAGCAGGTGTGACGGAT
SEQ ID NO. 111
(Rough (Ra or R) Core LPS Antigens R1F)
ATCCGTCACACCTGCTCTCCGCACGTAGGACCACTTTGGTACACGCTC
CCGTAGTGGTGTTGGCTCCCGTAT
SEQ ID NO. 112
(R5F)
ATCCGTCACACCTGCTCTACGGATGAACGAAGATTTTAAAGTCAAGCT
AATGCATGGTGTTGGCTCCCGTAT
SEQ ID NO. 113
(R6F)
ATCCGTCACACCTGCTCTGTAGTGAAGAGTCCGCAGTCCACGCTGTTC
AACTCATGGTGTTGGCTCCCGTAT
SEQ ID NO. 114
(R7F)
ATCCGTCACACCTGCTCTACCGGCTGGCACGGTTATGTGTGACGGGCG
AAGATATGGTGTTGGCTCCCGTAT
SEQ ID NO. 115
(R9F)
ATCCGTCACACCTGCTCTGCGTGTGGAGCGCCTAGGTGAGTGGTGTTG
GCTCCCGTAT
SEQ ID NO. 116
(R10F)
ATCCGTCACACCTGCTCTGATGTCCCTTTGAAGAGTTCCATGACGCTGG
CTCCTTGGTGTTGGCTCCCGTAT
SEQ ID NO. 117
(R1R)
ATACGGGAGCCAACACCACTACGGGAGCGTGTACCAAAGTGGTCCTA
CGTGCGGAGAGCAGGTGTGACGGAT
SEQ ID NO. 118
(R5R)
ATACGGGAGCCAACACCATGCATTAGCTTGACTTTAAAATCTTCGTTC
ATCCGTAGAGCAGGTGTGACGGAT
SEQ ID NO. 119
(R6R)
ATACGGGAGCCAACACCATGAGTTGAACAGCGTGGACTGCGGACTCTT
CACTACAGAGCAGGTGTGACGGAT
SEQ ID NO. 120
(R7R)
ATACGGGAGCCAACACCATATCTTCGCCCGTCACACATAACCGTGCCA
GCCGGTAGAGCAGGTGTGACGGAT
SEQ ID NO. 121
(R9R)
ATACGGGAGCCAACACCACTCACCTAGGCGCTCCACACGCAGAGCAG
GTGTGACGGAT
SEQ ID NO. 122
(R10R)
ATACGGGAGCCAACACCAAGGAGCCAGCGTCATGGAACTCTTCAAAG
GGACATCAGAGCAGGTGTGACGGAT
Enterococcus faecalis Teichoic Acid (TA) Aptamers
SEQ ID NO. 123
(TA5F)
CATTCACCACACCTCTGCTGGCTTGGCTAGCCTTGATGCTAAACGACCCA
TAGTGTGGTGTCGTCCCGTATC
SEQ ID NO. 124
(TA5R)
GATACGGGACGACACCACACTATGGGTCGTTTAGCATCAAGGCTAGCCAA
GCCAGCAGAGGTGTGGTGAATG
SEQ ID NO. 125
(TA6F)
CATTCACCACACCTCTGCTGGAGGAGGAAGTGGTCTGGAGTTACTTGACA
TAGTGTGGTGTCGTCCCGTATC
SEQ ID NO. 126
(TA6R)
GATACGGGACGACACCACACTATGTCAAGTAACTCCAGACCACTTCCTCC
TCCAGCAGAGGTGTGGTGAATG
SEQ ID NO. 127
(TA7F)
CATTCACCACACCTCTGCTGGACGGAAACAATCCCCGGGTACGAGAATCA
GGGTGTGGTGTCGTCCCGTATC
SEQ ID NO. 128
(TA7R)
GATACGGGACGACACCACACCCTGATTCTCGTACCCGGGGATTGTTTCCG
TCCAGCAGAGGTGTGGTGAATG
SEQ ID NO. 129
(TA9F)
CATTCACCACACCTCTGCTGGAAACCTACCATTAATGAGACATGATGCGG
TGGTGTGGTGTCGTCCCGTATC
SEQ ID NO. 130
(TA9R)
GATACGGGACGACACCACACCACCGCATCATGTCTCATTAATGGTAGGTT
TCCAGCAGAGGTGTGGTGAATG
Leishmania donovani and Leishmania tropica Surface
Protein Aptamers
SEQ ID NO. 131
(L-3F)
GATACGGGAGCCAACACCACCCGTATCGTTCCCAATGCACTCAGAGCAGG
TGTGACGGATG
SEQ ID NO. 132
(L-3R)
CATCCGTCACACCTGCTCTGAGTGCATTGGGAACGATACGGGTGGTGTTG
GCTCCCGTATG
SEQ ID NO. 133
(L-5F)
GATACGGGAGCCAACACCACGTTCCCATACAAGTTACTGACAGAGCAGGT
GTGACGGATG
SEQ ID NO. 134
(L-5R)
CATCCGTCACACCTGCTCTGTCAGTAACTTGTATGGGAACGTGGTGTTGG
CTCCCGTATC
Bacillus anthraces Poly-D-Glutamic Acid Capsule
Aptamers
SEQ ID NO. 135
(PDGA 2F)
CATCCGTCACACCTGCTCTGGTTCGCCCCGGTCAAGGAGAGTGGTGTTGG
CTCCCGTATC
SEQ ID NO. 136
(PDGA 2R)
GATACGGGAGCCAACACCACTCTCCTTGACCGGGGCGAACCAGAGCAGGT
GTGACGGATG
SEQ ID NO. 137
(PDGA 5F)
CATCCGTCACACCTGCTCTGGATAAGATCAGCAACAAGTTAGTGGTGTTG
GCTCCCGTATC
SEQ ID NO. 138
(PDGA 5R)
GATACGGGAGCCAACACCACTAACTTGTTGCTGATCTTATCAGAGCAGGT
GTGACGGATG
Organophosphorus Pesticide Aptamers
SEQ ID NO. 139
(Diazinon(D)12F)
ATACGGGAGCCAACACCATTAAATCAATTGTGCCGTGTTGGTCTTGTCTC
ATCGAGAGCAGGTGTGACGGAT
SEQ ID NO. 140
(D12R)
ATCCGTCACACCTGCTCTCGATGAGACAAGACCAACACGGCACAATTGAT
TTAATGGTGTTGGCTCCCGTAT
SEQ ID NO. 141
(D17F)
ATACGGGAGCCAACACCATTTTTATTATCGGTATGATCCTACGAGTTCCT
CCCAAGAGCAGGTGTGACGGAT
SEQ ID NO. 142
(D17R)
ATCCGTCACACCTGCTCTTGGGAGGAACTCGTAGGATCATACCGATAATA
AAAATGGTGTTGGCTCCCGTAT
SEQ ID NO. 143
(D18F)
ATACGGGAGCCAACACCACGTATATCTTATTATGCACAGCATCACGAA
AGTGC-AGAGCAGGTGTGACGGAT
SEQ ID NO. 144
(D18R)
ATCCGTCACACCTGCTCTTTTTTATTATCGGTATGATCCTACGAGTTCCT
CCCATGGTGTTGGCTCCCGTAT
SEQ ID NO. 145
(D19F)
ATACGGGAGCCAACACCATTAACGTTAAGCGGCCTCACTTCTTTTAATCC
TTTCAGAGCAGGTGTGACGGAT
SEQ ID NO. 146
(D19R)
ATCCGTCACACCTGCTCTGAAAGGATTAAAAGAAGTGAGGCCGCTTAACG
TTAATGGTGTTGGCTCCCGTAT
SEQ ID NO. 147
(D20F)
ATCCGTCACACCTGCTCTAATATAGAGGTATTGCTCTTGGACAAGGTACA
GGGATGGTGTTGGCTCCCGTAT
SEQ ID NO. 148
(D20R)
ATACGGGAGCCAACACCATCCCTGTACCTTGTCCAAGAGCAATACCTCTA
TATTACCACAACCGAGGGCATA
SEQ ID NO. 149
(Malathion(M)17F)
ATACGGGAGCCAACACCACAGTCAAGAAGTTAAGAGAAAAACAATTGTGT
ATAAGAGCAGGTGTGACGGAT
SEQ ID NO. 150
(M17R)
ATCCGTCACACCTGCTCTTATACACAATTGTTTTTCTCTTAACTTCTTGA
CTGCTGGTGTTGGCTCCCGTAT
SEQ ID NO. 151
(M21F)
ATCCGTCACACCTGCTCTGCGCCACAAGATTGCGGAAAGACACCCGGGGG
GCTTGGTGTTGGCTCCCGTAT
SEQ ID NO. 152
(M21R)
ATACGGGAGCCAACACCAGCCCCCCGGGTGTCTTTCCGCAATCTTGTGGC
GCAGAGCAGGTGTGACGGAT
SEQ ID NO. 153
(M25F)
ATCCGTCACACCTGCTCTGGCCTTATGTAAAGCGTTGGGTGGTGTTGGCT
CCCGTAT
SEQ ID NO. 154
(M25R)
ATACGGGAGCCAACACCACCCAACGCTTTACATAAGGCCAGAGCAGGTGT
GACGGAT
Foot-and-Mouth Disease (FMD) O-Serotype Viral
Capsid Aptamers
SEQ ID NO. 155
(FMD 1F)
ATACGGGAGCCAACACCATTCTATCGTTCCGGACGCTTATGCCITGCCAT
CTACAGAGCAGGTGTGACGGAT
SEQ ID NO. 156
(FMD 1R)
ATCCGTCACTCCTGCTCTGTAGATGGCAAGGCATAAGCGTCCGGAACGAT
AGAATGGTGTTGGCTCCCGTAT
SEQ ID NO. 157
(FMD 10F)
ATACGGGAGCCAACACCATGAATATCTCTTCTACCTCCTCTCCTCCCTT
TACTTAGAGCAGGTGTGACGGAT
SEQ ID NO. 158
(FMD 10R)
ATCCGTCACTCCTGCTCTAAGTAAAGGGAGGAGAGGAGGTAGAAGAGATA
TTCATGGTGTTGGCTCCCGTAT
SEQ ID NO. 159
(FMD 11F)
ATACGGGAGCCAACACCACGCCGCCCCAGTTCATGGCCTCTATGTCCGGC
AACGAGAGCAGGTGTGACGGAT
SEQ ID NO. 160
(FMD 11R)
ATCCGTCACTCCTGCTCTCGTTGCCGGACATAGAGGCCATGAACTGGGGC
GGCGTGGTGTTGGCTCCCGTAT
SEQ ID NO. 161
(FMD 12F)
ATACGGGAGCCAACACCATCTAGATCTGAAGAATAAAACAAAGACAAAGA
TGCTAGAGCAGGTGTGACGGAT
SEQ ID NO. 162
(FMD 12R)
ATCCGTCACTCCTGCTCTAGCATCTTTGTCTTTGTTTTATTCAGATCTAG
ATGGTGTTGGCTCCCGTAT
SEQ ID NO. 163
(FMD 13F)
ATACGGGAGCCAACACCACCTTTTAAAACGCTAGCTAGCTTAGTCCATTC
CACCAGAGCAGGTGTGACGGAT
SEQ ID NO. 164
(FMD 13R)
ATCCGTCACTCCTGCTCTGGTGGAATGGACTAAGCTAGCTAGCGTTTTAA
AAGGTGGTGTTGGCTCCCGTAT
Aptamer Sequences Against Markers of Bone
Resorption or Formation
SEQ ID NO. 165
(Hydroxylysine 5F)
ATACGGGAGCCAACACCACGCTTAGATATTATCCTTGTCCAGAGCAGG
TGTGACGGAT
SEQ ID NO. 166
(Hydroxylysine 5R)
ATCCGTCACACCTGCTCTGGACAAGGATAATATCTAAGCGTGGTGTTG
GCTCCCGTAT
SEQ ID NO. 167
(Hydroxylysine 6F)
ACACGGGAGCCAACACCATCCATAGCTCATCTATACCCTCTTCCGAGT
CCCACCAGAGCAGGTGTGACGGAT
SEQ ID NO. 168
(Hydroxylysine 6R)
ATCCGTCACACCTGCTCTGGTGGGACTCGGAAGAGGGTATAGATGAGC
TATGGATGGTGTTGGCTCCCGTGT
SEQ ID NO. 169
(Hydroxylysine 7F)
ATACGGGAGCCAACACCACCCTACACCAGCGCCCTACACTTTTGTAGC
ACTTCGAGAGCAGGTGTGACGGAT
SEQ ID NO. 170
(Hydroxylysine 7R)
ATCCGTCACACCTGCTCTCGAAGTGCTACAAAAGTGTAGGGCGCTGGT
GTAGGGTGGTGTTGGCTCCCGTAT
SEQ ID NO. 171
(Hydroxylysine 8F)
ATACGGGAGCCAACACCATAGTGTTGGGCCAATACGGTAACGTGTCCT
TGGAGAGCAGGTGTGACGGAT
SEQ ID NO. 172
(Hydroxylysine 8R)
ATCCGTCACACCTGCTCTCCAAGGACACGTTACCGTATTGGCCCAACA
CTATGGTGTTGGCTCCCGTAT
SEQ ID NO. 173
(Hydroxylysine 9F)
ATACGGGAGCCAACACCACCCGTTTTTGATCTAATGAGGATACAATAT
TCGTCTAGAGCAGGTGTGACGGAT
SEQ ID NO. 174
(Hydroxylysine 9R)
ATCCGTCACACCTGCTCTAGACGAATATTGTATCCTCATTAGATCAAA
AACGGGTGGTGTTGGCTCCCGTAT
SEQ ID NO. 175
(Hydroxylysine 10F)
ATACGGGAGCCAACACCAGCTTTTCCTAGAATGATTTTCTTTAGCTACC
TGAGAAGAGCAGGTGTGACGGAT
SEQ ID NO. 176
(Hydroxylysine 10R)
ATCCGTCACACCTGCTCTTCTCAGGTAGCTAAAGAAAATCATTCTAGG
AAAAGCTGGTGTTGGCTCCCGTAT
SEQ ID NO. 177
(Osteocalcin 2F)
ATACGGGAGCCAACACCACGATTAGCAATGAATTATCTACAGAGCAG
GTGTGACGGAT
SEQ ID NO. 178
(Osteocalcin 2R)
ATCCGTCACACCTGCTCTGTAGATAATTCATTGCTAATCGTGGTGTTGG
CTCCCGTAT
SEQ ID NO. 179
(Osteocalcin 4F)
ATACGGGAGCCAACACCAATTCTAACACAGGTTTCTCCGTTTCGTTAG
CTGCTAAGAGCAGGTGTGACGGAT
SEQ ID NO. 180
(Osteocalcin 4R)
ATCCGTCACACCTGCTCTTAGCAGCTAACGAAACGGAGAAACCTGTGT
TAGAATTGGTGTTGGCTCCCGTAT
SEQ ID NO. 181
(Osteocalcin 7F)
ATACGGGAGCCAACACCATTCTATCGTTCCGGACGCTTATGCCTTGCC
ATCTACAGAGCAGGTGTGACGGAT
SEQ ID NO. 182
(Osteocalcin 7R)
ATCCGTCACACCTGCTCTGTAGATGGCAAGGCATAAGCGTCCGGAACG
ATAGAATGGTGTTGGCTCCCGTAT
SEQ ID NO. 183
(Osteocalcin 8F)
ATACGGGAGCCAACACCAACTGACTCAGTCTGCTGGTGGGCTATATTT
TTGCGGAGAGCAGGTGTGACGGAT
SEQ ID NO. 184
(Osteocalcin 8R)
ATCCGTCACACCTGCTCTCCGCAAAAATATAGCCCACCAGCAGACTGA
GTCAGTTGGTGTTGGCTCCCGTAT
SEQ ID NO. 185
(ProCathepsin K 1F)
ATACGGGAGCCAACACCATATAGCCGCGCCTGTGAGTTTTGTGGGAGC
AAGAGTAGAGCAGGTGTGACGGAT
SEQ ID NO. 186
(ProCathepsin K 1R)
ATCCGTCACACCTGCTCTACTCTTGCTCCCACAAAACTCACAGGCGCG
GCTATATGGTGTTGGCTCCCGTAT
SEQ ID NO. 187
(ProCathepsin K 2F)
ATACGGGAGCCAACACCAGCTACAGTGTCAGACGGTTCCACCTTAACC
TCGTCAAGAGCAGGTGTGACGGAT
SEQ ID NO. 188
(ProCathepsin K 2R)
ATCCGTCACACCTGCTCTTGACGAGGTTAAGGTGGAACCGTCTGACAC
TGTAGCTGGTGTTGGCTCCCGTAT
SEQ ID NO. 189
(ProCathepsin K 3F)
ATACGGGAGCCAACACCATTGACTAAGCGATTAGTCCCACAGGTGAC
CGGGGAGAGAGCAGGTGTGACGGAT
SEQ ID NO. 190
(ProCathepsin K 3R)
ATCCGTCACACCTGCTCTCTTCCCGGNCTCCTGTGTGATTAATCTGTTA
TTCTATGGTGTTGGCTCCCGTAT
SEQ ID NO. 191
(ProCathepsin K 4F)
ATACGGGAGCCAACACCAATTCTAACACAGGTTTCTCCGTTTCGTTAG
CTGCTAAGAGCAGGTGTGACGGAT
SEQ ID NO. 192
(ProCathepsin K 4R)
ATCCGTCACACCTGCTCTTAGCAGCTAACGAAACGGAGAAACCTGTGT
TAGAATTGGTGTTGGCTCCCGTAT
SEQ ID NO. 193
(ProCathepsin K 6F)
ATACGGGAGCCAACACCACTCATCCCGTTGGAACACTTTAATATGGCC
CACTCTAGAGCAGGTGTGACGGAT
SEQ ID NO. 194
(ProCathepsin K 6R)
ATCCGTCACACCTGCTCTAGAGTGGGCCATATTAAAGTGTTCCAACGG
GATGAGTGGTGTTGGCTCCCGTAT
SEQ ID NO. 195
(C-Terminal Telopeptide of Human Collagen (CTx)
1F)
ATACGGGAGCCAACACCACTAACTTGTTGCTGATCTTATCCAGAGCAG
GTGTGACGGAT
SEQ ID NO. 196
(CTx 1R)
ATCCGTCACACCTGCTCTGGATAAGATCAGCAACAAGTTAGTGGTGTT
GGCTCCCGTAT
SEQ ID NO. 197
(CTx 2F)
ATACGGGAGCCAACACCACCCGTTTTTGATCTAATGAGGATACAATAT
TCGTCTAGAGCAGGTGTGACGGAT
SEQ ID NO. 198
(CTx 2R)
ATCCGTCACACCTGCTCTAGACGAATATTGTATCCTCATTAGATCAAA
AACGGGTGGTGTTGGCTCCCGTAT
SEQ ID NO. 199
(CTx 3F)
ATACGGGAGCCAACACCAATCGATGGTTAGACTATTACACTAGATGGA
ATTCATAGAGCAGGTGTGACGGAT
SEQ ID NO. 200
(CTx 3R)
ATCCGTCACACCTGCTCTATGAATTCCATCTAGTGTAATAGTCTAACCA
TCGATTGGTGTTGGCTCCCGTAT
SEQ ID NO. 201
(CTx 6F)
ATACGGGAGCCAACACCAATCTGCCGACTAGGCCAAGTAATTATATTC
AGCTGGAGAGCAGGTGTGACGGAT
SEQ ID NO. 202
(CTx 6R)
ATCCGTCACACCTGCTCTCCAGCTGAATATAATTACTTGGCCTAGTCGG
CAGATTGGTGTTGGCTCCCGTAT
SEQ ID NO. 203
(CTx 7F)
ATACGGGAGCCAACACCACAGCTGATATTGGATGGTCCGGCAGAGCA
GGTGTGACGGAT
SEQ ID NO. 204
(CTx 7R)
ATCCGTCACACCTGCTCTGCCGGACCATCCAATATCAGCTGTGGTGTT
GGCTCCCGTAT
SEQ ID NO. 205
(CTx 8F)
ATACGGGAGCCAACACCACAGCTGATATTGGATGGTCCGGCAGAGCA
GGTGTGACGGAT
SEQ ID NO. 206
(CTx 8R)
ATCCGTCACACCTGCTCTGCCGGACCATCCAATATCAGCTGTGGTGTT
GGCTCCCGTAT
SEQ ID NO. 207
(CTx 11F)
ATACGGGAGCCAACACCACATTACAATAGATGTATTGACATATCCGGA
CAGTCGAGAGCAGGTGTGACGGAT
SEQ ID NO. 208
(CTx 11R)
ATCCGTCACACCTGCTCTCGACTGTCCGGATATGTCAATACATCTATTG
TAATGTGGTGTTGGCTCCCGTAT
SEQ ID NO. 209
(CTx 13F)
ATACGGGAGCCAACACCACCCGTTTTTGATCTAATGAGGATACAATAT
TCGTCTAGAGCAGGTGTGACGGAT
SEQ ID NO. 210
(CTx 13R)
ATCCGTCACACCTGCTCTAGACGAATATTGTATCCTCATTAGATCAAA
AACGGGTGGTGTTGGCTCCCGTAT
SEQ ID NO. 211
(CTx 14F)
ATACGGGAGCCAACACCACTCGTGTAGTGCTGTCTTTGTGGAATCCTT
GCATCGAGAGCAGGTGTGACGGAT
SEQ ID NO. 212
(CTx 14R)
ATCCGTCACACCTGCTCTCGATGCAAGGATTCCACAAAGACAGCACTA
CACGAGTGGTGTTGGCTCCCGTAT
SEQ ID NO. 213
(CTx 15F)
ATACGGGAGCCAACACCACCACGTGACCCATACGATACAACAAATAA
TTGCTCAAGAGCAGGTGTGACGGAT
SEQ ID NO. 214
(CTx 15R)
ATCCGTCACACCTGCTCTTGAGCAATTATTTGTTGTATCGTATGGGTCA
CGTGGTGGTGTTGGCTCCCGTAT
SEQ ID NO. 215
(CTx 16F)
ATACGGGAGCCAACACCATCCATAGCTCATCTATACCCTCTTCCGAGT
CCCACCAGAGCAGGTGTGACGGAT
SEQ ID NO. 216
(CTx 16R)
ATCCGTCACACCTGCTCTGGTGGGACTCGGAAGAGGGTATAGATGAGC
TATGGATGGTGTTGGCTCCCGTAT
SEQ ID NO. 217
(CTx 17F)
ATACGGGAGCCAACACCAGACGCGGAACGACTCATCGCAAAATGTCG
TGATGCAAGAGCAGGTGTGACGGAT
SEQ ID NO. 218
(CTx 17R)
ATCCGTCACACCTGCTCTTGCATCACGACATTTTGCGATGAGTCGTTCC
GCGTCTGGTGTTGGCTCCCGTAT
SEQ ID NO. 219
(CTx 18F)
ATACGGGAGCCAACACCATGGTTAGGCTGCTCCATATATTCCCGCCCC
GCACGTAGAGCAGGTGTGACGGAT
SEQ ID NO. 220
(CTx 18R)
ATCCGTCACACCTGCTCTACGTGCGGGGCGGGAATATATGGAGCAGCC
TAACCATGGTGTTGGCTCCCGTAT
SEQ ID NO. 221
(CTx 19F)
ATACGGGAGCCAACACCACCCGTTTTTGATCTAATGAGGATACAATAT
TCGTCTAGAGCAGGTGTGACGGAT
SEQ ID NO. 222
(CTx 19R)
ATCCGTCACACCTGCTCTAGACGAATATTGTATCCTCATTAGATCAAA
AACGGGTGGTGTTGGCTCCCGTAT
SEQ ID NO. 223
(CTx 20F)
ATACGGGAGCCAACACCACCCGTTTTTGATCTTATGAGGATACAATAT
TCGTCTAGAGCAGGTGTGACGGAT
SEQ ID NO. 224
(CTx 20R)
ATCCGTCACACCTGCTCTAGACGAATATTGTATCCTCATAAGATCAAA
AACGGGTGGTGTTGGCTCCCGTAT
SEQ ID NO. 225
(N-Terminal Telopeptide of Human Collagen(NTx)
2F)
ATCCGTCACACCTGCTCTCCGACCAATGTGTGGATCATTACTAATCGACT
ATTGTGGTGTTGGCTCCCGTAT
SEQ ID NO. 226
(NTx 2R)
ATACGGGAGCCAACACCACAATAGTCGATTAGTAATGATCCACACATTGG
TCGGAGAGCAGGTGTGACGGAT
SEQ ID NO. 227
(NTx 4F)
ATACGGGAGCCAACACCATAGTTTTGGGCCAATACGGTAACGTGTCCTTG
GAGAGCAGGTGTGACGGAT
SEQ ID NO. 228
(NTx 4R)
ATCCGTCACACCTGCTCTCCAAGGACACGTTACCGTATTGGCCCAAAACT
ATGGTGTTGGCTCCCGTAT
SEQ ID NO. 229
(NTx 8F)
ATCCGTCACACCTGCTCTCCAAGGACACGTTACCGTATTGGTCCAGCACT
ATGGTGTTGGCTCCCGTAT
SEQ ID NO. 230
(NTx 8R)
ATACGGGAGCCAACACCATAGTGCTGGACCAATACGGTAACGTGTCCTTG
GAGAGCAGGTGTGACGGAT
SEQ ID NO. 231
(NTx 10F)
ATCCGTCACACCTGCTCTAACGTGTGGGTTGAAGTGTCGCCAACAAATTG
ATAGTGGTGTTGGCTCCCGTAT
SEQ ID NO. 232
(NTx 10R)
ATACGGGAGCCAACACCACTATCAATTTGTTGGCGACACTTCAACCCACA
CGTTAGAGCAGGTGTGACGGAT
SEQ ID NO. 233
(NTx 11F)
ATCCGTCACACCTGCTCTCGACCAAATATTTCCCCCAGCTCTAACCCATG
CTGATGGTGTTGGCTCCCGTAT
SEQ ID NO. 234
(NTx 11R)
ATACGGGAGCCAACACCATCAGCATGGGTTAGAGCTGGGGGAAATATTTG
GTCGAGAGCAGGTGTGACGGAT
SEQ ID NO. 235
(NTx 12F)
ATACGGGAGCCAACACCATAGTGTTGGGCCAATACGGTAACGTGTCCTTG
GAGAGCAGGTGTGACGGAT
SEQ ID NO. 236
(NTx 12R)
ATCCGTCACACCTGCTCTCCAAGGACACGTTACCGTATTGGCCCAACACT
ATGGTGTTGGCTCCCGTAT
SEQ ID NO. 237
(NTx 13F)
ATCCGTCACACCTGCTCTGGGGTCCGCTTGGGAACGATATTCCTGTTGTT
TTGTTGGTGTTGGCTCCCGTAT
SEQ ID NO. 238
(NTx 13R)
ATACGGGAGCCAACACCAACAAAACAACAGGAATATCGTTCCCAAGCGGA
CCCCAGAGCAGGTGTGACGGAT
SEQ ID NO. 239
(NTx 14F)
ATCCGTCACACCTGCTCTGATGGCAACATGGGTTAAATCTAACAACACTT
TGTATGGTGTTGGCTCCCGTAT
SEQ ID NO. 240
(NTx 14R)
ATACGGGAGCCAACACCATACAAAGTGTTGTTAGATTTAACCCATGTTGC
CATCAGAGCAGGTGTGACGGAT
SEQ ID NO. 241
(NTx 15F)
ATACGGGAGCCAACACCAAGGGTGTTCACACTGGCAGGCGACGCCCTCGT
GTTGAGAGCAGGTGTGACGGAT
SEQ ID NO. 242
(NTx 15R)
ATCCGTCACACCTGCTCTCAACACGAGGGCGTCGCCTGCCAGTGTGAACA
CCCTTGGTGTTGGCTCCCGTAT
SEQ ID NO. 243
(25-Hydroxy-Vitamin D3 (Calcidiol) VD3 1F)
ATACGGGAGCCAACACCATAGACAATGGCGTACTTTTCGTAATTCCAC
AAGAATAGAGCAGGTGTGACGGAT
SEQ ID NO. 244
(VD3 1R)
ATCCGTCACACCTGCTCTATTCTTGTGGAATTACGAAAAGTACGCCATT
GTCTATGGTGTTGGCTCCCGTAT
SEQ ID NO. 245
(VD3 2F)
ATACGGGAGCCAACACCACCACAAAAGCATTCGCCCTTACAGAGCAG
GTGTGACGGAT
SEQ ID NO. 246
(VD3 2R)
ATCCGTCACACCTGCTCTGTAAGGGCGAATGCTTTTGTGGTGGTGTTG
GCTCCCGTAT
SEQ ID NO. 247
(VD3 3F)
ATACGGGAGCCAACACCAGCGTGTAGCTAGTTTCAGGATTGTAGTATG
TAATATAGAGCAGGTGTGACGGAT
SEQ ID NO. 248
(VD3 3R)
ATCCGTCACACCTGCTCTATATTACATACTACAATCCTGAAACTAGCTA
CACGCTGGTGTTGGCTCCCGTAT
SEQ ID NO. 249
(VD3 5F)
ATACGGGAGCCAACACCACGCACATACTAGCTATCTCATCAGAGCAG
GTGTGACGGAT
SEQ ID NO. 250
(VD3 5R)
ATCCGTCACACCTGCTCTGATGAGATAGCTAGTATGTGCGTGGTGTTG
GCTCCCGTAT
SEQ ID NO. 251
(VD3 6F)
ATACGGGAGCCAACACCATCAGAGATCATCTAACGAAAATCATGGGT
CTCGCCCAGAGCAGGTGTGACGGAT
SEQ ID NO. 252
(VD3 6R)
ATCCGTCACACCTGCTCTGGGCGAGACCCATGATTTTCGTTAGATGAT
CTCTGATGGTGTTGGCTCCCGTAT
SEQ ID NO. 253
(VD3 7F)
ATACGGGAGCCAACACCAGCAAAGAATAGTGAGCCCTATGATCATCT
GTTCGTCAGAGCAGGTGTGACGGAT
SEQ ID NO. 254
(VD3 7R)
ATCCGTCACACCTGCTCTGACGAACAGATGATCATAGGGCTCACTATT
CTTTGCTGGTGTTGGCTCCCGTAT
SEQ ID NO. 255
(VD3 8F)
ATACGGGAGCCAACACCAGACATCATGTCGCATATCTGGATCTAGAGG
CTATTCAGAGCAGGTGTGACGGAT
SEQ ID NO. 256
(VD3 8R)
ATCCGTCACACCTGCTCTGAATAGCCTCTAGATCCAGATATGCGACAT
GATGTCTGGTGTTGGCTCCCGTAT
SEQ ID NO. 257
(VD3 10F)
ATACGGGAGCCAACACCAGTACGGCGGTGTCCGAACTCACTATACCC
AGTTGAAAGAGCAGGTGTGACGGAT
SEQ ID NO. 258
(VD3 10R)
ATCCGTCACACCTGCTCTTTCAACTGGGTATAGTGAGTTCGGACACCG
CCGTACTGGTGTTGGCTCCCGTAT
SEQ ID NO. 259
(VD3 13F)
ATACGGGAGCCAACACCAGACCTGACAACGAAAACCCCAGTTGTCGC
CATAGCCAGAGCAGGTGTGACGGAT
SEQ ID NO. 260
(VD3 13R)
ATCCGTCACACCTGCTCTGGCTATGGCGACAACTGGGGTTTTCGTTGTC
AGGTCTGGTGTTGGCTCCCGTAT
SEQ ID NO. 261
(VD3 14F)
ATACGGGAGCCAACACCATAGTGTTGGGCCAATACGGTAACGTGTCCT
TGGAGAGCAGGTGTGACGGAT
SEQ ID NO. 262
(VD3 14R)
ATCCGTCACACCTGCTCTCCAAGGACACGTTACCGTATTGGCCCAACA
CTATGGTGTTGGCTCCCGTAT
SEQ ID NO. 263
(VD3 15F)
ATACGGGAGCCAACACCATAAGCGCAACACAGTCCATCCCTGAGTGA
GATAGCGAGAGCAGGTGTGACGGAT
SEQ ID NO. 264
(VD3 15R)
ATCCGTCACACCTGCTCTCGCTATCTCACTCAGGGATGGACTGTGTTGC
GCTTATGGTGTTGGCTCCCGTAT
SEQ ID NO. 265
(VD3 16F)
ATACGGGAGCCAACACCACGCACATACTAGCTATCTCATCAGAGCAG
GTGTGACGGAT
SEQ ID NO. 266
(VD3 16R)
ATCCGTCACACCTGCTCTGATGAGATAGCTAGTATGTGCGTGGTGTTG
GCTCCCGTAT
SEQ ID NO. 267
(VD3 17F)
ATACGGGAGCCAACACCACTAACTTGTTGCTGATCTTACCAGAGCAGG
TGTGACGGAT
SEQ ID NO. 268
(VD3 17R)
ATCCGTCACACCTGCTCTGGTAAGATCAGCAACAAGTTAGTGGTGTTG
GCTCCCGTAT
SEQ ID NO. 269
(VD3 18F)
ATACGGGAGCCAACACCACCCGTTTTTGATCTAATGAGGATACAATAT
TCGTCNAGAGCAGGTGTGACGGAT
SEQ ID NO. 270
(VD3 18R)
ATCCGTCACACCTGCTCTNGACGAATATTGTATCCTCATTAGATCAAA
AACGGGTGGTGTTGGCTCCCGTAT
SEQ ID NO. 271
(VD3 19F)
ATACGGGAGCCAACACCAGTTGTGGGAACATCAGGCTAAGTATGAGA
CGGAACGAGAGCAGGTGTGACGGAT
SEQ ID NO. 272
(VD3 19R)
ATCCGTCACACCTGCTCTCGTTCCGTCTCATACTTAGCCTGATGTTCCC
ACAACTGGTGTTGGCTCCCGTAT
SEQ ID NO. 273
(VD3 20F)
ATACGGGAGCCAACACCATAGTGTTGGGCCAATACGGTAACGTGTCCT
TGGAGAGCAGGTGTGACGGAT
SEQ ID NO. 274
(VD3 20R)
ATCCGTCACACCTGCTCTCCAAGGACACGTTACCGTATTGGCCCAACA
CTATGGTGTTGGCTCCCGTAT
Botulinum Toxin Type A Aptamer Sequences
SEQ ID NO. 275
(Botulinum Toxin Type A-Light Chain (BoNT A-LC1))
CATCCGTCACACCTGCTCTGGGGATGTGTGGTGTTGGCTCCCGTATCAAG
GGCGAATTCT
SEQ ID NO. 276
(BoNT A-LC2)
GTAGGCAGTGTGGACGAGACCCCTACACACCACAACCGAGGGCATAGTTC
CCGCTTAAGA
SEQ ID NO.277
(Botulinum Toxin Type A-Holotoxin (BoNT A-HT1))
CATCCGTCACACCTGCTCTGCTATCACATGCCTGCTGAAGTGGTGTTGGC
TCCCGTATCA
SEQ ID NO. 278
(BoNT A-HT2)
GTAGGCAGTGTGGACGAGACGATAGTGTACGGACGACTTCACCACAACCG
AGGGCATAGT
E. coli Outer Membrane Proteins (OMPs)
SEQ ID NO. 279
(EcO-1F)
ATCCGTCACACCTGCTCTCGATGTCTGGGCCCTAATATTGGTTTGCTTGT
ACCATGGTGTTGGCTCCCGTAT
SEQ ID NO. 280
(EcO-1R)
ATACGGGAGCCAACACCATGGTACAAGCAAACCAATATTAGGGCCCAGAC
ATCGAGAGCAGGTGTGACGGAT
SEQ ID NO. 281
(EcO-2F)
ATACGGGAGCCAACACCATGATACCCTAAGGTAGGGGAGGCCTAAGCGCC
ACGTAGAGCAGGTGTGACGGAT
SEQ ID NO. 282
(EcO-2R)
ATCCGTCACACCTGCTCTACGTGGCGCTTAGGCCTCCCCTACCTTAGGGT
ATCATGGTGTTGGCTCCCGTAT
SEQ ID NO. 283
(EcO-3F)
ATACGGGAGCCAACACCACGCATCCCCCGCCGGGCCCGCGCCCCGCTCGC
AGACAGAGCAGGTGTGACGGAT
SEQ ID NO. 284
(EcO-3R)
ATCCGTCACACCTGCTCTGTCTGCGAGCGGGGCGCGGGCCCGGCGGGGGA
TGCGTGGTGTTGGCTCCCGTAT
SEQ ID NO. 285
(EcO-4F (73))
ATCCGTCACACCTGCTCTACGGCGCTCCCAACAGGCCTCTCCTTACGGCA
TATTATGGTGTTGGCTCCCGTAT
SEQ ID NO. 286
(EcO-4R (73))
ATACGGGAGCCAACACCATAATATGCCGTAAGGAGAGGCCTGTTGGGAGC
GCCGTAGAGCAGGTGTGACGGAT
SEQ ID NO. 287
(EcO-5F)
ATACGGGAGCCAACACCAGGAAAAAAAGAGCCTGTGAAGATTGTAATATC
AGTTAGAGCAGGTGTGACGGAT
SEQ ID NO. 288
(EcO-5R)
ATCCGTCACACCTGCTCTAACTGATATTACAATCTTCACAGGCTCTTTTT
TTCCTGGTGTTGGCTCCCGTAT
SEQ ID NO. 289
(EcO-7Fa)
ATCCGTCACACCTGCTCTCGGAGGTAGACTAGGATTGCGGCGGGGGGTCA
GGTATGGTGTTGGCTCCCGTAT
SEQ ID NO. 290
(EcO-7Fb)
ATACGGGAGCCAACACCACAAAAGCCTTACCTAACTGCCAACAATGAATA
GCAAGAGCAGGTGTGACGGAT
SEQ ID NO. 291
(EcO-7Ra)
ATCCGTCACACCTGCTCTTGCTATTCATTGTTGGCAGTTAGGTAAGGCTT
TTGTTGGTGTTGGCTCCCGTAT
SEQ ID NO. 292
(EcO-7Rb)
ATACGGGAGCCAACACCATACCTGACCCCCCGCCGCAATCCTAGTCTACC
TCCGAGAGCAGGTGTGACGGAT
SEQ ID NO. 293
(EcO-8F)
ATACGGGAGCCAACACCACGACTAACACGACCGTTGGGGGGGGCTCGCGC
GGGCAGAGCAGGTGTGACGGAT
SEQ ID NO. 294
(EcO-8R)
ATCCGTCACACCTGCTCTGCCCGCGCGAGCCCCCCCCAACGGTCGTGTTA
GTCGTGGTGTTGGCTCCCGTAT
SEQ ID NO. 295
(EcO-9F)
ATACGGGAGCCAACACCAGTCCCCGCCCAGCCGTGAGCCGTACCCCCGCA
CACCAGAGCAGGTGTGACGGAT
SEQ ID NO. 296
(EcO-9R)
ATCCGTCACACCTGCTCTGGTGTGCGGGGGTACGGCTCACGGCTGGGCGG
GGACTGGTGTTGGCTCCCGTAT
SEQ ID NO. 297
(EcO-10F)
ATCCGTCACACCTGCTCTCAAGGTTGGGCCTGCAAGAGCAAAAACGGGGC
GGGATGGTGTTGGCTCCCGTAT
SEQ ID NO. 298
(EcO-10R)
ATACGGGAGCCAACACCATCCCGCCCCGTTTTTGCTCTTGCAGGCCCAAC
CTTGAGAGCAGGTGTGACGGAT
SEQ ID NO. 299
(EcO-11F)
ATCCGTCACACCTGCTCTACTTGGCTTGCGACTATTATTCACAGGGCCAA
AGACTGGTGTTGGCTCCCGTAT
SEQ ID NO. 300
(EcO-11 R)
ATACGGGAGCCAACACCAGTCTTTGGCCCTGTGAATAATAGTCGCAAGCC
AAGTAGAGCAGGTGTGACGGAT
SEQ ID NO. 301
(EcO-12F (69))
ATACGGGAGCCAACACCATAGTGTTGGACCAATACGGTAACGTGTCCTTG
GAGAGCAGGTGTGACGGAT
SEQ ID NO. 302
(EcO-12R (69))
ATCCGTCACACCTGCTCTCCAAGGACACGTTACCGTATTGGTCCAACACT
ATGGTGTTGGCTCCCGTAT
SEQ ID NO. 303
(EcO-17F)
ATCCGTCACACCTGCTCTTGGAATGTCGGTGTTTTTCCAATTCCTTGGGT
CGTGTGGTGTTGGCTCCCGTAT
SEQ ID NO. 304
(EcO-17R)
ATACGGGAGCCAACACCA CACGACCCAAGGAATTGGAAAAACACCGACA
TTCCAAGAGCAGGTGTGACGGAT
SEQ ID NO. 305
(EcO-18F)
ATCCGTCACACCTGCTCTGCGACGGCGACGCGGTCCGGGCGGGGGTGGAG
GACGTGGTGTTGGCTCCCGTAT
SEQ ID NO. 306
(EcO-18R)
ATACGGGAGCCAACACCACGTCCTCCACCCCCGCCCGGACCGCGTCGCCG
TCGCAGAGCAGGTGTGACGGAT
SEQ ID NO. 307
(EcO-19Fa)
ATACGGGAGCCAACACCAGAGGGTTCTAGGGTCACTTCCATGAGAATGGC
TCACAGAGCAGGTGTGACGGAT
SEQ ID NO. 308
(EcO-19Fb)
ATCCGTCACACCTGCTCTGGCCTGGGGACGCGAGGGAGGCGGGGGGAGTC
GTGGTGGTGTTGGCTCCCGTAT
SEQ ID NO. 309
(EcO-19Ra)
ATACGGGAGCCAACACCACCACGACTCCCCCCGCCTCCCTCGCGTCCCCA
GGCCAGAGCAGGTGTGACGGAT
SEQ ID NO. 310
(EcO-19Rb)
ATCCGTCACACCTGCTCT GTGAGCCATTCTCATGGAAGTGACCCTAGAA
CCCTCTGGTGTTGGCTCCCGTAT
SEQ ID NO. 311
(EcO-20F)
ATCCGTCACACCTGCTCTCACAGGGCCTCTTACTATACAGTTCTCCAGCG
CTGCTGGTGTTGGCTCCCGTAT
SEQ ID NO. 312
(EcO-20R)
ATACGGGAGCCAACACCAGCAGCGCTGGAGAACTGTATAGTAAGAGGCCC
TGTGAGAGCAGGTGTGACGGAT
SEQ ID NO. 313
(EcO-21F)
ATCCGTCACACCTGCTCTGCACGGGCTCAGTTTGGCTTTGTATCCTAAGA
GAGATGGTGTTGGCTCCCGTAT
SEQ ID NO. 314
(EcO-21R)
ATACGGGAGCCAACACCATCTCTCTTAGGATACAAAGCCAAACTGAGCCC
GTGCAGAGCAGGTGTGACGGAT
SEQ ID NO. 315
(EcO-22F)
ATACGGGAGCCAACACCAGGGGTGGCGAACATGGTATAACTTGATAAGTG
TGAAGAGCAGGTGTGACGGAT
SEQ ID NO. 316
(EcO-22R)
ATCCGTCACACCTGCTCTTCACACTTATCAAGTTATACCATGTTCGCCAC
CCCCTGGTGTTGGCTCCCGTAT
SEQ ID NO. 317
(EcO-23F)
ATACGGGAGCCAACACCACTCCGACACCGGCCGCCGGCACCACCCACTCC
CCCTAGAGCAGGTGTGACGGAT
SEQ ID NO. 318
(EcO-23R)
ATCCGTCACACCTGCTCTAGGGGGAGTGGGTGGTGCCGGCGGCCGGTGTC
GGAGTGGTGTTGGCTCCCGTAT
SEQ ID NO. 319
(EcO-24F)
ATACGGGAGCCAACACCATCCGGCGCGCCCTCCTCCCCCACTGCTCCCCG
CCCGAGAGCAGGTGTGACGGAT
SEQ ID NO. 320
(EcO-24R)
ATCCGTCACACCTGCTCTCGGGCGGGGAGCAGTGGGGGAGGAGGGCGCGC
CGGATGGTGTTGGCTCCCGTAT
SEQ ID NO. 321
(EcO-25F)
ATACGGGAGCCAACACCATACGCAGAGGTCCCCTACCCAGGCCAGCCGGA
TGCCAGAGCAGGTGTGACGGAT
SEQ ID NO. 322
(EcO-25R)
ATCCGTCACACCTGCTCTGGCATCCGGCTGGCCTGGGTAGGGGACCTCTG
CGTATGGTGTTGGCTCCCGTAT
Shiga toxins (Shiga-like Toxin type 1; Stx-1)
SEQ ID NO. 323
(SH-2F)
ATCCGTCACACCTGCTCTGGAGACATTAAAAACCGGAGTTTATTTATACC
TTTCTGGTGTTGGCTCCCGTAT
SEQ ID NO. 324
(SH-2R)
ATACGGGAGCCAACACCAGAAAGGTATAAATAAACTCCGGTTTTTAATGT
CTCCAGAGCAGGTGTGACGGAT
SEQ ID NO. 325
(SH-3F (59))
ATACGGGAGCCAACACCACTAACTTGTTGCTGATCTTATCCAGAGCAGGT
GTGACGGAT
SEQ ID NO. 326
(SH-3R (59))
ATCCGTCACACCTGCTCTGGATAAGATCAGCAACAAGTTAGTGGTGTTGG
CTCCCGTAT
SEQ ID NO. 327
(SH-4F (58))
ATCCGTCACACCTGCTCTGCATGGAGAGTTTTTTGGTCAGTGGTGTTGGC
TCCCGTAT
SEQ ID NO. 328
(SH-4R (58))
ATACGGGAGCCAACACCACTGACCAAAAAACTCTCCATGCAGAGCAGGTG
TGACGGAT
SEQ ID NO. 329
(SH-6F (58))
ATACGGGAGCCAACACCACGTTAACGCGTAGCCTTTGGACAGAGCAGGTG
TGACGGAT
SEQ ID NO. 330
(SH-6R (58))
ATCCGTCACACCTGCTCTGTCCAAAGGCTACGCGTTAACGTGGTGTTGGC
TCCCGTAT
SEQ ID NO. 331
(SH-8/21/23/24/25F (59))
ATCCGTCACACCTGCTCTGCCGGACCATCCAATATCAGCTGTGGTGTTGG
CTCCCGTAT
SEQ ID NO. 332
(SH-8/21/23/24/25 Rev (59))
ATACGGGAGCCAACACCACAGCTGATATTGGATGGTCCGGCAGAGCAGGT
GTGACGGAT
SEQ ID NO. 333
(SH-9F)
ATCCGTCACACCTGCTCTCGTCCGTCATTAAGTTCGGAGGCTGGCGGGTT
GCGTTGGTGTTGGCTCCCGTAT
SEQ ID NO. 334
(SH-9R)
ATACGGGAGCCAACACCAACGCAACCCGCCAGCCTCCGAACTTAATGACG
GACGAGAGCAGGTGTGACGGAT
SEQ ID NO. 335
(SH-10F)
ATACGGGAGCCAACACCATTCTATCGTTCCGGACGCTTATGCCTTGCCAT
CTACAGAGCAGGTGTGACGGAT
SEQ ID NO. 336
(SH-10R)
ATCCGTCACACCTGCTCTGTAGATGGCAAGGCATAAGCGTCCGGAACGAT
AGAATGGTGTTGGCTCCCGTAT
SEQ ID NO. 337
(SH-11F)
TCCGTCACACCTGCTCTAACTCTTACTACTTTGTTGCTATCACATTCAAC
TGTTGGTGTTGGCTCCCGTAT
SEQ ID NO. 338
(SH-11R)
ATACGGGAGCCAACACCAACAGTTGAATGTGATAGCAACAAAGTAGTAAG
AGTTAGAGCAGGTGTGACGGAT
SEQ ID NO. 339
(SH-12 F(58))
ATCCGTCACACCTGCTCTGGCCTTTCACCAAGCGTCCTTGTGGTGTTGGC
TCCCGTAT
SEQ ID NO. 340
(SH-12R (58))
ATACGGGAGCCAACACCACAAGGACGCTTGGTGAAAGGCCAGAGCAGGT
GTGACGGAT
SEQ ID NO. 341
(SH-16F (58))
ATCCGTCACACCTGCTCTGGCACCGAGCACGGGAACCCAGTGGTGTTGGC
TCCCGTAT
SEQ ID NO. 342
(SH-16R (58))
ATACGGGAGCCAACACCACTGGGTTCCCGTGCTCGGTGCCAGAGCAGGTG
TGACGGAT
SEQ ID NO. 343
(SH-17F (69))
ATACGGGAGCCAACACCATAGTGTTGGGCCAATACGGTAACGTGTCCTT
GGAGAGCAGGTGTGACGGAT
SEQ ID NO. 344
(SH-17R (69))
ATCCGTCACACCTGCTCTCCAAGGACACGTTACCGTATTGGCCCAACACT
ATGGTGTTGGCTCCCGTAT
SEQ ID NO. 345
(SH-18F)
ATCCGTCACACCTGCTCTACCCGATGCCGCCCCGGGATTGTTGTATGACC
ATCTTGGTGTTGGCTCCCGTAT
SEQ ID NO. 346
(SH-18R)
ATACGGGAGCCAACACCAAGATGGTCATACAACAATCCCGGGGCGGCATC
GGGTAGAGCAGGTGTGACGGAT
SEQ ID NO. 347
(SH-19F)
ATACGGGAGCCAACACCACCCCATGAGTACACGTGAACGGACACAGCCTC
CGGCAGAGCAGGTGTGACGGAT
SEQ ID NO. 348
(SH-19R)
ATCCGTCACACCTGCTCTGCCGGAGGCTGTGTCCGTTCACGTGTACTCAT
GGGGTGGTGTTGGCTCCCGTAT
SEQ ID NO. 349
(SH-20F)
ATCCGTCACACCTGCTCTTAACCATTCATTTCTTTTGTGGTATGACCGTT
CGCCTGGTGTTGGCTCCCGTAT
SEQ ID NO. 350
(SH-20R)
ATACGGGAGCCAACACCAGGCGAACGGTCATACCACAAAAGAAATGAATG
GTTAAGAGCAGGTGTGACGGAT
SEQ ID NO. 351
(SH-22F (58))
ATCCGTCACACCTGCTCTGGGGCTCTTTTCGTTAACCAGGTGGTGTTGGC
TCCCGTAT
SEQ ID NO. 352
(SH-22R (58))
ATACGGGAGCCAACACCACCTGGTTAACGAAAAGAGCCCCAGAGCAGGTG
TGACGGAT
S. typhimurium (S. enterica serovar Typhimurium
type 13311) OMPs
SEQ ID NO. 353
(StO-2F)
ATACGGGAGCCAACACCAGATAAATTTTGCGTTCATTCTTATTTCCTGT
CCGCCAGAGCAGGTGTGACGGAT
SEQ ID NO. 354
(StO-2R)
ATCCGTCACACCTGCTCTGGCGGACAGGAAATAAGAATGAACGCAAA
ATTTATCTGGTGTTGGCTCCCGTAT
SEQ ID NO. 355
(StO-4F)
ATACGGGAGCCAACACCAGATAAATTTTGGTTCATTCTTATTTCCTGTC
CGCCAGAGCAGGTGTGACGGAT (71)
SEQ ID NO. 356
(StO-4R)
ATCCGTCACACCTGCTCTGGCGGACAGGAAATAAGAATGAACCAAAA
TTTATCTGGTGTTGGCTCCCGTAT (71)
SEQ ID NO. 357
(StO-5F)
ATACGGGAGCCAACACCACGGGGCTACCAGCACCGTCACCCCTCATTC
TGCCACAGAGCAGGTGTGACGGAT
SEQ ID NO. 358
(StO-5R)
ATCCGTCACACCTGCTCTGTGGCAGAATGAGGGGTGACGGTGCTGGTA
GCCCCGTGGTGTTGGCTCCCGTAT
SEQ ID NO. 359
(StO-6F)
ATACGGGAGCCAACACCAAAAGATGGAAAACACTGGAAGGAAAATGC
GGTCAGAGCAGGTGTGACGGAT (69)
SEQ ID NO. 360
(StO-6R)
ATCCGTCACACCTGCTCTGACCGCATTTTCCTTCCAGTGTTTTCCATCTT
TTGGTGTTGGCTCCCGTAT (69)
SEQ ID NO. 361
(StO-7F)
ATACGGGAGCCAACACCACCGGGCCGATGGGCACCAGGAACTCTCGG
ACGAGTGAGAGCAGGTGTGACGGAT
SEQ ID NO. 362
(StO-7R)
ATCCGTCACACCTGCTCTCACTCGTCCGAGAGTTCCTGGTGCCCATCG
GCCCGGTGGTGTTGGCTCCCGTAT
SEQ ID NO. 363
(StO-8F)
ATACGGGAGCCAACACCACAGCTGATATTGGATGGTCCGGCAGAGCA
GGTGTGACGGAT (59)
SEQ ID NO. 364
(StO-8R)
ATCCGTCACACCTGCTCTGCCGGACCATCCAATATCAGCTGTGGTGTT
GGCTCCCGTAT (59)
SEQ ID NO. 365
(StO-9F)
ATACGGGAGCCAACACCAGTCGAAAGGCGGCCGTCCAGTCGAGTGAT
TTGACCTAGAGCAGGTGTGACGGAT
SEQ ID NO. 366
(StO-9R)
ATCCGTCACACCTGCTCTAGGTCAAATCACTCGACTGGACGGCCGCCT
TTCGACTGGTGTTGGCTCCCGTAT
SEQ ID NO. 367
(StO-10F)
ATACGGGAGCCAACACCACGGGGCGTGCCGTCAAAAGACCGAGATGT
GGCTGCGAGAGCAGGTGTGACGGAT
SEQ ID NO. 368
(StO-10R)
ATCCGTCACACCTGCTCTCGCAGCCACATCTCGGTCTTTTGACGGCAC
GCCCCGTGGTGTTGGCTCCCGTAT
SEQ ID NO. 369
(StO-11/13F)
ATACGGGAGCCAACACCACTAACTTGTTGCTGATCTTATCCAGAGCAG
GTGTGACGGAT (59)
SEQ ID NO. 370
(StO-11/13R)
ATCCGTCACACCTGCTCTGGATAAGATCAGCAACAAGTTAGTGGTGTT
GGCTCCCGTAT (59)
SEQ ID NO. 371
(StO-12F)
ATACGGGAGCCAACACCATTTAGCGTAGGGCTCGCTTATCATTTCTCA
TTCCCTAGAGCAGGTGTGACGGAT
SEQ ID NO. 372
(StO-12R)
ATCCGTCACACCTGCTCTAGGGAATGAGAAATGATAAGCGAGCCCTAC
GCTAAATGGTGTTGGCTCCCGTAT
SEQ ID NO. 373
(StO-14F)
ATACGGGAGCCAACACCACCGCAACCCAAATCTCTACACGGATTATCG
TCGAGCAGAGCAGGTGTGACGGAT
SEQ ID NO. 374
(StO-14R)
ATCCGTCACACCTGCTCTGCTCGACGATAATCCGTGTAGAGATTTGGG
TTGCGGTGGTGTTGGCTCCCGTAT
SEQ ID NO. 375
(StO-16F)
ATACGGGAGCCAACACCAACACATTCTATAATGAATGTTCCTGTCGCG
TTGCGTAGAGCAGGTGTGACGGAT
SEQ ID NO. 376
(StO-16R)
ATCCGTCACACCTGCTCTACGCAACGCGACAGGAACATTCATTATAGA
ATGTGTTGGTGTTGGCTCCCGTAT
SEQ ID NO. 377
(StO-17F)
ATACGGGAGCCAACACCAGCCTACCCCCCCTGTACGAGGGCCGCAAC
CACGTAGAGAGCAGGTGTGACGGAT
SEQ ID NO. 378
(StO-17R)
ATCCGTCACACCTGCTCTCTACGTGGTTGCGGCCCTCGTACAGGGGGG
GTAGGCTGGTGTTGGCTCCCGTAT
SEQ ID NO. 379
(StO-18F)
ATACGGGAGCCAACACCACATCTAGCACGAGACCCTATCCCAGAGCA
GGTGTGACGGAT(59)
SEQ ID NO. 380
(StO-18R)
ATCCGTCACACCTGCTCTGGGATAGGGTCTCGTGCTAGATGTGGTGTT
GGCTCCCGTAT(59)
SEQ ID NO. 381
(StO-19F)
ATACGGGAGCCAACACCAACAGCGACTCGAGTCTGACGACTCGCGGG
GCAAATGAGAGCAGGTGTGACGGAT
SEQ ID NO. 382
(StO-19R)
ATCCGTCACACCTGCTCTCATTTGCCCCGCGAGTCGTCAGACTCGAGT
CGCTGTTGGTGTTGGCTCCCGTAT
SEQ ID NO. 383
(StO-20/24F)
ATACGGGAGCCAACACCATAGTGTTGGGCCAATACGGTAACGTGTCCT
TGGAGAGCAGGTGTGACGGAT (69)
SEQ ID NO. 384
(StO-20/24R)
ATCCGTCACACCTGCTCTCCAAGGACACGTTACCGTATTGGCCCAACA
CTATGGTGTTGGCTCCCGTAT (69)
SEQ ID NO. 385
(StO-21F)
ATACGGGAGCCAACACCACTAAGGAGAGGTCGCGACAGACTCTTCTG
GTCAAGGAGAGCAGGTGTGACGGAT
SEQ ID NO. 386
(StO-21R)
ATCCGTCACACCTGCTCTCCTTGACCAGAAGAGTCTGTCGCGACCTCT
CCTTAGTGGTGTTGGCTCCCGTATG
SEQ ID NO. 387
(StO-22F)
ATACGGGAGCCAACACCAACTTCGACTCAAAGAAGTCCACGTGAGAC
TGGTGGAAGAGCAGGTGTGACGGAT
SEQ ID NO. 388
(StO-22R)
ATCCGTCACACCTGCTCTTCCACCAGTCTCACGTGGACTTCTTTGAGTC
GAAGTTGGTGTTGGCTCCCGTAT
SEQ ID NO. 389
(StO-23F)
ATACGGGAGCCAACACCACCCGGGGAGACCCGCACGGGCGCACAATC
CTTGTCGAGAGCAGGTGTGACGGAT
SEQ ID NO. 390
(StO-23R)
ATCCGTCACACCTGCTCTCGACAAGGATTGTGCGCCCGTGCGGGTCTC
CCCGGGTGGTGTTGGCTCCCGTAT
SEQ ID NO. 391
(StO-25F)
ATACGGGAGCCAACACCAGCTGGACCAAACTACGCCCATTGTGGGGG
TCCCCGGAGAGCAGGTGTGACGGAT
SEQ ID NO. 392
(StO-25R)
ATCCGTCACACCTGCTCTCCGGGGACCCCCACAATGGGCGTAGTTTGGTC
CAGCTGGTGTTGGCTCCCGTAT
Acetylcholine (ACh)
SEQ ID NO. 393
(ACh1aF)
ATACGGGAGCCAACACCACGATACCCGCTTATGAATTTTAAATTAATTGT
GATCAGAGCAGGTGTGACGGAT
SEQ ID NO. 394
(ACh 1aR)
ATCCGTCACACCTGCTCTGATCACAATTAATTTAAAATTCATAAGCGGGT
ATCGTGGTGTTGGCTCCCGTAT
SEQ ID NO. 395
(ACh 1bF)
ATACGGGAGCCAACACCAACTTTCACACATACTTGTTATACCACACGATC
TTTTAGAGCAGGTGTGACGGAT
SEQ ID NO. 396
(ACh 1bR)
ATCCGTCACACCTGCTCTAAAAGATCGTGTGGTATAACAAGTATGTGTGA
AAGTTGGTGTTGGCTCCCGTAT
SEQ ID NO. 397
(ACh 2F)
ATACGGGAGCCAACACCACTTTGTAACTCATTTGTAGTTTGGGTTGCTCC
CCCTAGAGCAGGTGTGACGGAT
SEQ ID NO. 398
(ACh 2R)
ATCCGTCACACCTGCTCTAGGGGGAGCAACCCAAACTACAAATGAGTTAC
AAAGTGGTGTTGGCTCCCGTAT
SEQ ID NO. 399
(ACh 3F)
ATACGGGAGCCAACACCATTTCCCGCTTATCTTCATCCACTGCTTAGCAT
ATGTAGAGCAGGTGTGACGGAT
SEQ ID NO. 400
(ACh 3R)
ATCCGTCACACCTGCTCTACATATGCTAAGCAGTGGATGAAGATAAGCGG
GAAATGGTGTTGGCTCCCGTAT
SEQ ID NO. 401
(ACh 5F)
ATACGGGAGCCAACACCAGGCACTGTATCACACCGTCAAGAATGTGATCC
CCTGAGAGCAGGTGTGACGGAT
SEQ ID NO. 402
(ACh 5R)
ATCCGTCACACCTGCTCTCAGGGGATCACATTCTTGACGGTGTGATACAG
TGCCTGGTGTTGGCTCCCGTAT
SEQ ID NO. 403
(ACh 6F)
ATACGGGAGCCAACACCATGTCATTTACCTTCATCATGACAGTGTTAGTA
TACGAGAGCAGGTGTGACGGAT
SEQ ID NO. 404
(ACh 6R)
ATCCGTCACACCTGCTCTAGGGGATCAAAGCTATGCGACCATGCGAGTGG
ATACTGGTGTTGGCTCCCGTAT
SEQ ID NO. 405
(ACh 7F)
ATACGGGAGCCAACACCAGTTGCCGCCTACCTTGATTATTCTACATTACC
CGTTAGAGCAGGTGTGACGGAT
SEQ ID NO. 406
(ACh 7R)
ATCCGTCACACCTGCTCTAACGGGTAATGTAGAATAATCAAGGTAGGCGG
CAACTGGTGTTGGCTCCCGTAT
SEQ ID NO. 407
(ACh 8F)
ATACGGGAGCCAACACCAGTATACATACGAAGAGTTGAAACCAATGCTTC
GTTCAGAGCAGGTGTGACGGAT
SEQ ID NO. 408
(ACh 8R)
ATCCGTCACACCTGCTCTGAACGAAGCATTGGTTTCAACTCTTCGTATGT
ATACTGGTGTTGGCTCCCGTAT
SEQ ID NO. 409
(ACh 9F)
ATACGGGAGCCAACACCATACCCCGAATGGCTGTTTTCAGTACCAATATG
ACTCAGAGCAGGTGTGACGGAT
SEQ ID NO. 410
(ACh 9R)
ATCCGTCACACCTGCTCTGAGTCATATTGGTACTGAAAACAGCCATTCGG
GGTATGGTGTTGGCTCCCGTAT
SEQ ID NO. 411
(ACh 10F)
ATACGGGAGCCAACACCACTGTCACGATCGTCGTTCCTTTTAATCCTTGT
GTCTAGAGCAGGTGTGACGGAT
SEQ ID NO. 412
(ACh 10R)
ATCCGTCACACCTGCTCTAGACACAAGGATTAAAAGGAACGACGATCGTG
ACAGTGGTGTTGGCTCCCGTAT
SEQ ID NO. 413
(ACh 11F)
ATACGGGAGCCAACACCACTGGACACTGACCCTCGCACTAGCTTTCTGAC
GGGTAGAGCAGGTGTGACGGAT
SEQ ID NO. 414
(ACh 11 R)
ATCCGTCACACCTGCTCTACCCGGCCGAAGAATAGTGCTCGGTACTTAGT
CGCGTGGTGTTGGCTCCCGTAT
SEQ ID NO. 415
(ACh 12F)
ATACGGGAGCCAACACCATTTGGACTTTAAATAGTGGACTCCTTCTTTGT
CTCGAGAGCAGGTGTGACGGAT
SEQ ID NO. 416
(ACh 12R)
ATCCGTCACACCTGCTCTCGAGACAAAGAAGGAGTCCACTATTTAAAGTC
CAAATGGTGTTGGCTCCCGTAT
Gram Negative Quorum Sensing Molecules (N-
Acylhomoserine Lactones; AHLs)
SEQ ID NO. 417
(Dec AHL 1F)
ATACGGGAGCCAACACCATCCTAACTGGTCTAATTTTTGCTGTTACCGAT
CCCGAGAGCAGGTGTGACGGAT
SEQ ID NO. 418
(Dec AHL 1R)
ATCCGTCACTCCTGCTCTCGGGATCGGTAACAGCAAAAATTAGACCAGTT
AGGATGGTGTTGGCTCCCGTAT
SEQ ID NO. 419
(Dec AHL 13F)
ATACGGGAGCCAACACCAGCCTGACGAAAAAATTTTATCACTAAGTGATA
CGCAAGAGCAGGTGTGACGGAT
SEQ ID NO. 420
(Dec AHL 13R)
ATCCGTCACACCTGCTCTTGCGTATCACTTAGTGATAAAATTTTTTCGTC
AGGCTGGTGTTGGCTCCCGTAT
SEQ ID NO. 421
(Dec AHL 14F)
ATACGGGAGCCAACACCAGACCTACTTCAGAAACGGAAATGTTCTTAGCC
GTCAGAGCAGGTGTGACGGAT
SEQ ID NO. 422
(Dec AHL 14R)
ATCCGTCACACCTGCTCTGACGGCTAAGAACATTTCCGTTTCTGAAGTAG
GTCTGGTGTTGGCTCCCGTAT
SEQ ID NO. 423
(Dec AHL 15F)
ATACGGGAGCCAACACCAGGCCAACGAAACTCCTACTACATATAATGCTT
ATGCAGAGCAGGTGTGACGGAT
SEQ ID NO. 424
(Dec AHL 15R)
ATCCGTCACACCTGCTCTGCATAAGCATTATATGTAGTAGGAGTTTCGTT
GGCCTGGTGTTGGCTCCCGTAT
SEQ ID NO. 425
(Dec AHL 17F)
ATACGGGAGCCAACACCATCCTAACTGGTCTAATTTTTGCTGTTACCGAT
CCCGAGAGCAGGTGTGACGGAT
SEQ ID NO. 426
(Dec AHL 17R)
ATCCGTCACACCTGCTCTCGGGATCGGTAACAGCAAAAATTAGACCAGTT
AGGATGGTGTTGGCTCCCGTAT

I claim:

1. A method of a sandwich assay, run by producing and assembling DNA or RNA aptamer-magnetic bead conjugates, for the capture and detection of a target analyte in a bulk solution, comprising:
combining said bulk solution, an aptamer-magnetic bead conjugate (“aptamer-MB”), and an aptamer-fluorophore conjugate in a cuvette, wherein said aptamer-MB is able to bind with said target analyte at a first binding site and said aptamer-fluorophore conjugate is able to bind with said target analyte at a second binding site to form an analyte-aptamer-fluorophore complex, and wherein said cuvette has a translucent surface area so as to enable a fluorescent assay;
allowing said aptamer-MB to bind with said target analyte at said first binding site and said aptamer-fluorophore conjugate to bind with said target analyte at said second binding site to form said analyte-aptamer-fluorophore complex;
adhering said analyte-aptamer-fluorophore complex to said cuvette translucent surface area by applying an external magnetic field to attract said magnetic bead; and
assaying said analyte-aptamer-fluorophore complex that is adhered to said cuvette translucent surface area.

2. The method of claim 1 wherein said method does not include a wash step.

3. The method of claim 1 wherein said analyte-aptamer-fluorophore complex is effectively partitioned away from said bulk solution to enhance detectability.

4. The method of claim 1 wherein said cuvette is made from polystyrene, clear plastic, or glass.

5. The method of claim 4 in which said cuvette translucent surface area, on which said analyte-aptamer-fluorophore complex adheres, is formed as a square, rectangular, round, oval, or flat container, vial, tube, cylinder, cassette, or cartridge.

6. The method of claim 1, wherein said aptamer-MB and said aptamer-fluorophore will not bind, base pair, or hybridize with each other in said bulk solution.

7. The method of claim 1, wherein said fluorophore in said aptamer-fluorophore conjugate is a quantum dot (“QD”), fluorescent or phosphorescent nanoparticle (“NP”), a fluorescent latex particle or microbead, a fluorescent dye molecule, such as fluoroescein, carboxyfluorescein and a fluorescein derivative, or a rhodamine or its derivatives.

8. The method of claim 1 in which said fluorophore is a fluorescence resonance energy transfer (“FRET”) complex such as an intrachain or a competitive FRET-aptamer.

9. The method of claim 1, wherein said assaying step is a sandwich assay to detect and quantify said target analyte in said bulk solution.

10. The method of claim 1, wherein said target analyte is a whole cell, such as a bacterium, parasite, leukocyte, or cancer cell.

11. The method of claim 1, wherein said target analyte is a protein, viral capsid protein, viral polymerase, biotoxin such as bacterial toxin, such as botulinum, cholera, tetnus, staphylococcal enterotoxin, shigatoxins or verotoxins, algal toxin, such as brevetoxin, ciguatoxin, cyanotoxin, or saxitoxin, snake or spider venom, clinically relevant protein or portions of protein (peptides) such as bone marker (e.g., collagen breakdown peptides such as CTx, NTx, OCF, Cathepsin K or its precursor ProCathepsin K, deoxypyridinoline, pyridinoline, lysyl pyridinoline, or hydroxylysyl pyridinoline) cytokines and interleukins, markers of myocardial infarctions (troponin, myoglobin, etc.), kidney disease, antibodies, autoimmune disorders, arthritis, or other clinically relevant macromolecules such as lipopolysaccharides (LPS, endotoxins).

12. The method of claim 1, wherein said target analyte includes small molecules (molecules of less than 1,000 Daltons) with at least two distinct epitopes from a group including the following: pesticides, natural and synthetic amino acids and their derivatives, hydroxylysine, hydroxyproline, histidine, histamine, homocysteine, DOPA, melatonin, nitrotyrosine, short chain proteolysis products, cadaverine, putrescine, polyamines, spermine, spermidine, deoxypyridinoline, pyridinoline, lysyl pyridinoline, or hydroxylysyl pyridinoline, nitrogen bases of DNA or RNA, nucleosides, nucleotides, nucleotide cyclical isoforms, cAMP, cGMP, cellular metabolites, urea, uric acid, pharmaceuticals, therapeutic drugs, vitamins, illegal drugs, narcotics, hallucinogens, gamma-hydroxybutyrate (GHB), cellular mediators, cytokines, chemokines, immune modulators, neural modulators, neurotransmitters such as acetylcholine, inflammatory modulators, prostaglandins, prostaglandin metabolites, nitoaromatic and nitramine explosives, explosive breakdown products (e.g., DNT) or byproducts, quorum sensing molecules such as AHLs, steroids, hormones, and their derivatives.

13. The method of claim 1 in which said assaying step of a target analyte is performed using one of: fluorescence intensity, time-resolved fluorescence, chemiluminescence, electrical detection, electrochemical detection, electrochemiluminescence, phosphorescence, or radioisotopic detection.


SEQUENCE LISTING (426 sequences)

SEQ ID NO: 1 - DNA, 62 bp, Artificial Sequence
  chemically synthesized
catccgtcacacctgctctggggagggtggcgcccgtctcggtggtgttggctcccgtatca

SEQ ID NO: 2 - DNA, 62 bp, Artificial Sequence
  chemically synthesized
catccgtcacacctgctctgggatagggtctcgtgctagatgtggtgttggctcccgtatca

SEQ ID NO: 3 - DNA, 62 bp, Artificial Sequence
  chemically synthesized
catccgtcacacctgctctggaccggcgcttattcctgcttgtggtgttggctcccgtatca

SEQ ID NO: 4 - DNA, 62 bp, Artificial Sequence
  chemically synthesized
catccgtcacacctgcyctggagctgatattggatggtccggtggtgttggctcccgtatca

SEQ ID NO: 5 - DNA, 62 bp, Artificial Sequence
  chemically synthesized
catccgtcacacctgcycygcccagagcaggtgtgacggatgtggtgttggctcccgtatca

SEQ ID NO: 6 - DNA, 62 bp, Artificial Sequence
  chemically synthesized
catccgtcacacctgcycygccggaccatccaatatcagctgtggtgttggctcccgtatca

SEQ ID NO: 7 - DNA, 72 bp, Artificial Sequence
  chemically synthesized
atccgtcacacctgctctggtggaatggactaagctagctagcgttttaaaaggtggtgttggctcccgtat

SEQ ID NO: 8 - DNA, 71 bp, Artificial Sequence
  chemically synthesized
atccgtcacacctgctctgtaagggggggaatcgctttcgtcttaagatgacatggtgttggctcccgtat

SEQ ID NO: 9 - DNA, 59 bp, Artificial Sequence
  chemically synthesized
atccgtcacacctgctctgccggaccatccaatatcagctgtggtgttggctcccgtat

SEQ ID NO: 10 - DNA, 71 bp, Artificial Sequence
  chemically synthesized
atccgtcacacctgctctatccgtcacgcctgctctatccgtcacacctgctctggtgttggctcccgtat

SEQ ID NO: 11 - DNA, 72 bp, Artificial Sequence
  chemically synthesized
atccgtcacacctgctctatcaaatgtgcagatatcaagacgatttgtacaagatggtgttggctcccgtat

SEQ ID NO: 12 - DNA, 72 bp, Artificial Sequence
  chemically synthesized
atccgtcacacctgctctgtagatggcaaggcataagcgtccggaacgatagaatggtgttggctcccgtat

SEQ ID NO: 13 - DNA, 70 bp, Artificial Sequence
  chemically synthesized
atccgtcacacctgctctgtagatggcaaggcatgcgtccggaacgatagaatggtgttggctcccgtat

SEQ ID NO: 14 - DNA, 72 bp, Artificial Sequence
  chemically synthesized
atacgggagccaacaccaccttttaaaacgctagctagcttagtccattccaccagagcaggtgtgacggat

SEQ ID NO: 15 - DNA, 72 bp, Artificial Sequence
  chemically synthesized
atacgggagccaacaccatgtcatcttaagacgaaagcgattccccccccttacagagcaggtgtgacggat

SEQ ID NO: 16 - DNA, 59 bp, Artificial Sequence
  chemically synthesized
atacgggagccaacaccacagctgatattggatggtccggcagagcaggtgtgacggat

SEQ ID NO: 17 - DNA, 71 bp, Artificial Sequence
  chemically synthesized
atacgggagccaacaccagagcaggtgtgacggatagagcaggcgtgacggatagagcaggtgtgacggat

SEQ ID NO: 18 - DNA, 72 bp, Artificial Sequence
  chemically synthesized
atacgggagccaacaccatcttgtacaaatcgtcttgatatctgcacatttgatagagcaggtgtgacggat

SEQ ID NO: 19 - DNA, 72 bp, Artificial Sequence
  chemically synthesized
atacgggagccaacaccattctatcgttccggacgcttatgccttgccatctacagagcaggtgtgacggat

SEQ ID NO: 20 - DNA, 72 bp, Artificial Sequence
  chemically synthesized
atacgggagccaacaccattctatcgttccggacgcttatgccttgccatctcacgagcaggtgtgacggat

SEQ ID NO: 21 - DNA, 59 bp, Artificial Sequence; identical to SEQ ID NO: 9
  chemically synthesized

SEQ ID NO: 22 - DNA, 72 bp, Artificial Sequence; identical to SEQ ID NO: 7
  chemically synthesized

SEQ ID NO: 23 - DNA, 72 bp, Artificial Sequence
  chemically synthesized
atccgtcacacctgctcttaaagtagaggctgttctccagacgtcgcaggaggatggtgttggctcccgtat

SEQ ID NO: 24 - DNA, 72 bp, Artificial Sequence; identical to SEQ ID NO: 12
  chemically synthesized

SEQ ID NO: 25 - DNA, 72 bp, Artificial Sequence; identical to SEQ ID NO: 12
  chemically synthesized

SEQ ID NO: 26 - DNA, 59 bp, Artificial Sequence; identical to SEQ ID NO: 16
  chemically synthesized

SEQ ID NO: 27 - DNA, 72 bp, Artificial Sequence
  chemically synthesized
atccgtcacacctgctcttgggcaggagcgagagactctaatggtaagcaagaatggtgttggctcccgtat

SEQ ID NO: 28 - DNA, 72 bp, Artificial Sequence
  chemically synthesized
atccgtcacacctgctctccaacaaggcgaccgaccgcatgcagatagccaggttggtgttggctcccgtat

SEQ ID NO: 29 - DNA, 59 bp, Artificial Sequence; identical to SEQ ID NO: 16
  chemically synthesized

SEQ ID NO: 30 - DNA, 72 bp, Artificial Sequence; identical to SEQ ID NO: 14
  chemically synthesized

SEQ ID NO: 31 - DNA, 72 bp, Artificial Sequence
  chemically synthesized
atacgggagccaacaccatcctcctgcgacgtctggagaacagcctctactttaagagcaggtgtgacggat

SEQ ID NO: 32 - DNA, 72 bp, Artificial Sequence; identical to SEQ ID NO: 19
  chemically synthesized

SEQ ID NO: 33 - DNA, 72 bp, Artificial Sequence; identical to SEQ ID NO: 19
  chemically synthesized

SEQ ID NO: 34 - DNA, 58 bp, Artificial Sequence
  chemically synthesized
atccgtcacacctgctctgccggaccatccaatatcagctgtggtgttggctccgtat

SEQ ID NO: 35 - DNA, 72 bp, Artificial Sequence
  chemically synthesized
atacgggagccaacaccattcttgcttaccattagagtctctcgctcctgcccaagagcaggtgtgacggat

SEQ ID NO: 36 - DNA, 71 bp, Artificial Sequence
  chemically synthesized
atacgggagccacaccaacctggctatctgcatgcggtcggtcgccttgttggagagcagttgtgacggat

SEQ ID NO: 37 - DNA, 72 bp, Artificial Sequence; identical to SEQ ID NO: 12
  chemically synthesized

SEQ ID NO: 38 - DNA, 72 bp, Artificial Sequence
  chemically synthesized
atccgtcacacctgctctaaccaaaagggtaggagaccaagctagcgatttggatggtgttggctcccgtat

SEQ ID NO: 39 - DNA, 59 bp, Artificial Sequence; identical to SEQ ID NO: 9
  chemically synthesized

SEQ ID NO: 40 - DNA, 72 bp, Artificial Sequence
  chemically synthesized
atccgtcacacctgctctgaagcctaacggagaagatggccctactgccgtaggtggtgttggctcccgtat

SEQ ID NO: 41 - DNA, 72 bp, Artificial Sequence
  chemically synthesized
atccgtcacacctgctctactaaacaagggcaaactgtaaacacagtaggggcgtggtgttggctcccgtat

SEQ ID NO: 42 - DNA, 68 bp, Artificial Sequence
  chemically synthesized
atccgtcacacctgctctggtgttggctcccgtatagcttggctcccgtatggtgttggctcccgtat

SEQ ID NO: 43 - DNA, 72 bp, Artificial Sequence
  chemically synthesized
atccgtcacacctgctgtctcgcgatgatgagcagcagcgcaggagggagggggtggtgttggctcccgtat

SEQ ID NO: 44 - DNA, 57 bp, Artificial Sequence
  chemically synthesized
atccgtcacacctgctctgatcagggaagacgccaacactggtgttggctcccgtat

SEQ ID NO: 45 - DNA, 72 bp, Artificial Sequence
  chemically synthesized
atacgggagccaacaccattctatcgttccggacgcttatgccttgccatctacagagcagttgtgacggat

SEQ ID NO: 46 - DNA, 72 bp, Artificial Sequence
  chemically synthesized
atacgggagccaacaccatccaaatcgctagcttggtctcctacccttttggttagagcaggtgtgacggat

SEQ ID NO: 47 - DNA, 59 bp, Artificial Sequence; identical to SEQ ID NO: 16
  chemically synthesized

SEQ ID NO: 48 - DNA, 72 bp, Artificial Sequence
  chemically synthesized
atacgggagccaacaccacctacggcagtagggccatcttctccgttaggcttcagagcaggtgtgacggat

SEQ ID NO: 49 - DNA, 72 bp, Artificial Sequence
  chemically synthesized
atacgggagccaacaccacgcccctactgtgtttacagtttgcccttgtttagtagagcaggtgtgacggat

SEQ ID NO: 50 - DNA, 68 bp, Artificial Sequence
  chemically synthesized
atacgggagccaacaccatacgggagccaagctatacgggagccaacaccagagcaggtgtgacggat

SEQ ID NO: 51 - DNA, 72 bp, Artificial Sequence
  chemically synthesized
atacgggagccaacaccaccccctccctcctgcgctgctgctcatcatcgcgacagagcaggtgtgacggat

SEQ ID NO: 52 - DNA, 57 bp, Artificial Sequence
  chemically synthesized
atacgggagccaacaccagtgttggcgtcttccctgatcagagcaggtgtgacggat

SEQ ID NO: 53 - DNA, 58 bp, Artificial Sequence
  chemically synthesized
atccgtcacacctgctctgtccaaaggctacgcgttaacgtggtgttggctcccgtat

SEQ ID NO: 54 - DNA, 58 bp, Artificial Sequence
  chemically synthesized
atccgtcacacctgctctggagcaatatggtggagaaacgtggtgttggctcccgtat

SEQ ID NO: 55 - DNA, 59 bp, Artificial Sequence; identical to SEQ ID NO: 9
  chemically synthesized

SEQ ID NO: 56 - DNA, 72 bp, Artificial Sequence
  chemically synthesized
atccgtcacacctgctctgaacaggatagggattagcgagtcaactaagcagcatggtgttggctcccgtat

SEQ ID NO: 57 - DNA, 72 bp, Artificial Sequence
  chemically synthesized
atccgtcacacctgctctggcggacaggaaataagaatgaacgcaaaatttatctggtgttggctcccgtat

SEQ ID NO: 58 - DNA, 72 bp, Artificial Sequence
  chemically synthesized
atccgtcacacctgctctacgcaacgcgacaggaacattcattatagaatgtgttggtgttggctcccgtat

SEQ ID NO: 59 - DNA, 72 bp, Artificial Sequence
  chemically synthesized
atccgtcacacctgctctcggctgcaatgcgggagagtaggggggaaccaaacctggtgttggctcccgtat

SEQ ID NO: 60 - DNA, 72 bp, Artificial Sequence
  chemically synthesized
atccgtcacacctgctctatgactggaacacgggtatcgatgattagatgtccttggtgttggctcccgtat

SEQ ID NO: 61 - DNA, 58 bp, Artificial Sequence
  chemically synthesized
atacgggagccaacaccacgttaacgcgtagcctttggacagagcaggtgtgacggat

SEQ ID NO: 62 - DNA, 58 bp, Artificial Sequence
  chemically synthesized
tatcgggagccaacaccacgtttctccaccatattgctccagagcaggtgtgacggat

SEQ ID NO: 63 - DNA, 59 bp, Artificial Sequence; identical to SEQ ID NO: 16
  chemically synthesized

SEQ ID NO: 64 - DNA, 72 bp, Artificial Sequence
  chemically synthesized
atacgggagccaacaccatgctgcttagttgactcgctaatccctatcctgttcagagcaggtgtgacggat

SEQ ID NO: 65 - DNA, 72 bp, Artificial Sequence; identical to SEQ ID NO: 64
  chemically synthesized

SEQ ID NO: 66 - DNA, 72 bp, Artificial Sequence
  chemically synthesized
atacgggagccaacaccaacacattctataatgaatgttcctgtcgcgttgcgtagagcaggtgtgacggat

SEQ ID NO: 67 - DNA, 72 bp, Artificial Sequence
  chemically synthesized
atacgggagccaacaccaggtttggttcccccctactctcccgcattgcagccgagagcaggtgtgacggat

SEQ ID NO: 68 - DNA, 72 bp, Artificial Sequence
  chemically synthesized
atacgggagccaacaccaaggacatctaatcatcgatacccgtgttccagtcatagagcaggtgtgacggat

SEQ ID NO: 69 - DNA, 72 bp, Artificial Sequence
  chemically synthesized
atccgtcacacctgctctaattaggatacggggcaacagaacgagaggggggaatggtgttggctcccgtat

SEQ ID NO: 70 - DNA, 71 bp, Artificial Sequence
  chemically synthesized
atccgtcacacctgctctcggaccaggtcagacaagcacatcggatatccggctggtgttggctcccgtat

SEQ ID NO: 71 - DNA, 72 bp, Artificial Sequence
  chemically synthesized
atccgtcacacctgctcttgagtcaaagagtttagggaggagctaacataacagtggtgttggctcccgtat

SEQ ID NO: 72 - DNA, 71 bp, Artificial Sequence
  chemically synthesized
atccgtcacacctgctctaacaacaatgcatcagcgggctgggacgcatgcggtggtgttggctcccgtat

SEQ ID NO: 73 - DNA, 72 bp, Artificial Sequence
  chemically synthesized
atccgtcacacctgctctgaacaggttataagcaggagtgatagtttcaggatctggtgttggctcccgtat

SEQ ID NO: 74 - DNA, 71 bp, Artificial Sequence
  chemically synthesized
atccgtcacacctgctctcggcggctcgcaaaccgagtggtcagcacccgggttggtgttggctcccgtat

SEQ ID NO: 75 - DNA, 72 bp, Artificial Sequence
  chemically synthesized
atccgtcacacctgctctgcgcaagacgtaatccacaagaccgtgaaaacatagtggtgttggctcccgtat

SEQ ID NO: 76 - DNA, 72 bp, Artificial Sequence
  chemically synthesized
atacgggagccaacaccattcccccctctcgttctgttgccccgtatcctaattagagcaggtgtgacggat

SEQ ID NO: 77 - DNA, 71 bp, Artificial Sequence
  chemically synthesized
atacgggagccaacaccagccggatatccgatgtgcttgtctgacctggtccgagagcaggtgtgacggat

SEQ ID NO: 78 - DNA, 72 bp, Artificial Sequence
  chemically synthesized
atacgggagccaacaccactgttatgttagctcctccctaaactctttgactcaagagcaggtgtgacggat

SEQ ID NO: 79 - DNA, 72 bp, Artificial Sequence
  chemically synthesized
atacgggagccaacaccaccgcatgcgttcccagcccgctgatgcattgttgttagagcaggtgtgacggat

SEQ ID NO: 80 - DNA, 72 bp, Artificial Sequence
  chemically synthesized
atacgggagccaacaccagatcctgaaactatcactcctgcttataacgtgttcagagcaggtgtgacggat

SEQ ID NO: 81 - DNA, 71 bp, Artificial Sequence
  chemically synthesized
atacgggagccaacaccaacccgggtgctgaccactcggtttgcgagccgccgagagcaggtgtgacggat

SEQ ID NO: 82 - DNA, 72 bp, Artificial Sequence
  chemically synthesized
atacgggagccaacaccactatgttttcacggtcttgtggattacgtcttgcgcagagcaggtgtgacggat

SEQ ID NO: 83 - DNA, 72 bp, Artificial Sequence
  chemically synthesized
atccgtcacacctgctgtaggcgtagtgactaagtcgcgcgaaaatcacagcattggtgttggctcccgtat

SEQ ID NO: 84 - DNA, 71 bp, Artificial Sequence
  chemically synthesized
atccgtcaccctgctctcagcggcagctatacagtgagaacggactagtgcgttggtgttggctcccgtat

SEQ ID NO: 85 - DNA, 72 bp, Artificial Sequence
  chemically synthesized
atccgtcacacctgctctggcaaataatactagcgatgatggatctggatagactggtgttggctcccgtat

SEQ ID NO: 86 - DNA, 72 bp, Artificial Sequence
  chemically synthesized
atccgtcacacctgctctgggggtgcgacttagggtaagtgggaaagacgatgctggtgttggctcccgtat

SEQ ID NO: 87 - DNA, 72 bp, Artificial Sequence
  chemically synthesized
atccgtcacacctgctctcaagaggagatgaaccaatcttagtccgacaggcggtggtgttggctcccgtat

SEQ ID NO: 88 - DNA, 72 bp, Artificial Sequence
  chemically synthesized
atccgtcacacctgctctggcccggaattgtcatgacgtcacctacacctcctgtggtgttggctcccgtat

SEQ ID NO: 89 - DNA, 72 bp, Artificial Sequence
  chemically synthesized
atacgggagccaacaccaatgctgtgattttcgcgcgacttagtcactacgcctagagcaggtgtgacggat

SEQ ID NO: 90 - DNA, 72 bp, Artificial Sequence
  chemically synthesized
atacgggagccaacaccaacgcactagtccgttctcactgtatagctgccgctgagagcaggtgtgacggat

SEQ ID NO: 91 - DNA, 72 bp, Artificial Sequence
  chemically synthesized
atacgggagccaacaccagtctatccagatccatcatcgctagtattatttgccagagcaggtgtgacggat

SEQ ID NO: 92 - DNA, 72 bp, Artificial Sequence
  chemically synthesized
atacgggagccaacaccagcatcgtctttcccacttaccctaagtcgcacccccagagcaggtgtgacggat

SEQ ID NO: 93 - DNA, 72 bp, Artificial Sequence
  chemically synthesized
atacgggagccaacaccaccgcctgtcggactaagattggttcatctcctcttgagagcaggtgtgacggat

SEQ ID NO: 94 - DNA, 72 bp, Artificial Sequence
  chemically synthesized
atacgggagccaacaccacaggaggtgtaggtgacgtcatgacaattccgggccagagcaggtgtgacggat

SEQ ID NO: 95 - DNA, 72 bp, Artificial Sequence
  chemically synthesized
atccgtcacccctgctctcgtcgctatgaagtaacaaagataggagcaatcgggtggtgttggctcccgtat

SEQ ID NO: 96 - DNA, 72 bp, Artificial Sequence
  chemically synthesized
atccgtcacacctgctctaacgaagactgaaaccaaagcagtgacagtgctgaatggtgttggctcccgtat

SEQ ID NO: 97 - DNA, 72 bp, Artificial Sequence
  chemically synthesized
atccgtcacacctgctctcggtgacaatagctcgatcagcccaaagtcgtcagatggtgttggctcccgtat

SEQ ID NO: 98 - DNA, 71 bp, Artificial Sequence
  chemically synthesized
atccgtcacacctgctctaacgaaatagaccacaaatcgatactttatgttattggtgttggctcccgtat

SEQ ID NO: 99 - DNA, 72 bp, Artificial Sequence
  chemically synthesized
atccgtcacacctgctctgtcgaatgctctgcctggaagagttgttagcagggatggtgttggctcccgtat

SEQ ID NO: 100 - DNA, 72 bp, Artificial Sequence
  chemically synthesized
atccgtcacacctgctcttaagccgaggggtaaatctaggacaggggtccatgatggtgttggctcccgtat

SEQ ID NO: 101 - DNA, 72 bp, Artificial Sequence
  chemically synthesized
atccgtcacacctgctctactggccggctcagcatgactaagaaggaagttatgtggtgttggctcccgtat

SEQ ID NO: 102 - DNA, 72 bp, Artificial Sequence
  chemically synthesized
atccgtcacacctgctctggtacgaatcacaggggatgctggaagcttggctcttggtgttggctcccgtat

SEQ ID NO: 103 - DNA, 72 bp, Artificial Sequence
  chemically synthesized
atacgggagccaacaccacccgattgctcctatctttgttacttcatagcgacgagagcaggggtgacggat

SEQ ID NO: 104 - DNA, 72 bp, Artificial Sequence
  chemically synthesized
atacgggagccaacaccattcagcactgtcactgctttggtttcagtcttcgttagagcaggtgtgacggat

SEQ ID NO: 105 - DNA, 72 bp, Artificial Sequence
  chemically synthesized
atacgggagccaacaccatctgacgactttgggctgatcgagctattgtcaccgagagcaggtgtgacggat

SEQ ID NO: 106 - DNA, 71 bp, Artificial Sequence
  chemically synthesized
atacgggagccaacaccaataacataaagtatcgatttgtggtctatttcgttagagcaggtgtgacggat

SEQ ID NO: 107 - DNA, 72 bp, Artificial Sequence
  chemically synthesized
atacgggagccaacaccatccctgctaacaactcttccaggcagagcattcgacagagcaggtgtgacggat

SEQ ID NO: 108 - DNA, 72 bp, Artificial Sequence
  chemically synthesized
atacgggagccaacaccatcatggacccctgtcctagatttacccctcggcttaagagcaggtgtgacggat

SEQ ID NO: 109 - DNA, 72 bp, Artificial Sequence
  chemically synthesized
atacgggagccaacaccacataacttccttcttagtcatgctgagccggccagtagagcaggtgtgacggat

SEQ ID NO: 110 - DNA, 72 bp, Artificial Sequence
  chemically synthesized
atacgggagccaacaccaagagccaagcttccagcatcccctgtgattcgtaccagagcaggtgtgacggat

SEQ ID NO: 111 - DNA, 72 bp, Artificial Sequence
  chemically synthesized
atccgtcacacctgctctccgcacgtaggaccactttggtacacgctcccgtagtggtgttggctcccgtat

SEQ ID NO: 112 - DNA, 72 bp, Artificial Sequence
  chemically synthesized
atccgtcacacctgctctacggatgaacgaagattttaaagtcaagctaatgcatggtgttggctcccgtat

SEQ ID NO: 113 - DNA, 72 bp, Artificial Sequence
  chemically synthesized
atccgtcacacctgctctgtagtgaagagtccgcagtccacgctgttcaactcatggtgttggctcccgtat

SEQ ID NO: 114 - DNA, 72 bp, Artificial Sequence
  chemically synthesized
atccgtcacacctgctctaccggctggcacggttatgtgtgacgggggaagatatggtgttggctcccgtat

SEQ ID NO: 115 - DNA, 58 bp, Artificial Sequence
  chemically synthesized
atccgtcacacctgctctgcgtctggagcgcctaggtgagtggtgttggctcccgtat

SEQ ID NO: 116 - DNA, 72 bp, Artificial Sequence
  chemically synthesized
atccgtcacacctgctctgatgtccctttgaagagttccatgacgctggctccttggtgttggctcccgtat

SEQ ID NO: 117 - DNA, 72 bp, Artificial Sequence
  chemically synthesized
atacgggagccaacaccactacgggagcgtgtaccaaagtggtcctacgtgcggagagcaggtgtgacggat

SEQ ID NO: 118 - DNA, 72 bp, Artificial Sequence
  chemically synthesized
atacgggagccaacaccatgcattagcttgactttaaaatcttcgttcatccgtagagcaggtgtgacggat

SEQ ID NO: 119 - DNA, 72 bp, Artificial Sequence
  chemically synthesized
atacgggagccaacaccatgagttgaacagcgtggactgcggactcttcactacagagcaggtgtgacggat

SEQ ID NO: 120 - DNA, 72 bp, Artificial Sequence
  chemically synthesized
atacgggagccaacaccatatcttcgcccgtcacacataaccgtgccagccggtagagcaggtgtgacggat

SEQ ID NO: 121 - DNA, 58 bp, Artificial Sequence
  chemically synthesized
atacgggagccaacaccactcacctaggcgctccacacgcagagcaggtgtgacggat

SEQ ID NO: 122 - DNA, 72 bp, Artificial Sequence
  chemically synthesized
atacgggagccaacaccaaggagccagcgtcatggaactcttcaaagggacatcagagcaggtgtgacggat

SEQ ID NO: 123 - DNA, 72 bp, Artificial Sequence
  chemically synthesized
cattcaccacacctctgctggcttggctagccttgatgctaaacgacccatagtgtggtgtcgtcccgtatc

SEQ ID NO: 124 - DNA, 72 bp, Artificial Sequence
  chemically synthesized
gatacgggacgacaccacactatgggtcgtttagcatcaaggctagccaagccagcagaggtgtggtgaatg

SEQ ID NO: 125 - DNA, 71 bp, Artificial Sequence
  chemically synthesized
catcaccacacctctgctggaggaggaagtggtctggagttacttgacatagtgtggtgtcgtcccgtatc

SEQ ID NO: 126 - DNA, 72 bp, Artificial Sequence
  chemically synthesized
gatacgggacgacaccacactatgtcaagtaactccagaccacttcctcctccagcagaggtgtggtgaatg

SEQ ID NO: 127 - DNA, 72 bp, Artificial Sequence
  chemically synthesized
cattcaccacacctctgctggacggaaacaatccccgggtacgagaatcagggtgtggtgtcgtcccgtatc

SEQ ID NO: 128 - DNA, 72 bp, Artificial Sequence
  chemically synthesized
gatacgggacgacaccacaccctgattctcgtacccggggattgtttccgtccagcagaggtgtggtgaatg

SEQ ID NO: 129 - DNA, 72 bp, Artificial Sequence
  chemically synthesized
cattcaccacacctctgctggaaacctaccattaatgagacatgatgcggtggtgtggtgtcgtcccgtatc

SEQ ID NO: 130 - DNA, 71 bp, Artificial Sequence
  chemically synthesized
gatacgggacgacaccacaccaccgcatcatgtctcattaatggtaggttccagcagaggtgtggtgaatg

SEQ ID NO: 131 - DNA, 61 bp, Artificial Sequence
  chemically synthesized
gatacgggagccaacaccacccgtatcgttcccaatgcactcagagcaggtgtgacggatg

SEQ ID NO: 132 - DNA, 61 bp, Artificial Sequence
  chemically synthesized
catccgtcacacctgctctgagtgcattgggaacgatacgggtggtgttggctcccgtatg

SEQ ID NO: 133 - DNA, 60 bp, Artificial Sequence
  chemically synthesized
gatacgggagccaacaccacgttcccatacaagttactgacagagcaggtgtgacggatg

SEQ ID NO: 134 - DNA, 60 bp, Artificial Sequence
  chemically synthesized
catccgtcacacctgctctgtcagtaacttgtatgggaacgtggtgttggctcccgtatc

SEQ ID NO: 135 - DNA, 60 bp, Artificial Sequence
  chemically synthesized
catccgtcacacctgctctggttcgccccggtcaaggagagtggtgttggctcccgtatc

SEQ ID NO: 136 - DNA, 60 bp, Artificial Sequence
  chemically synthesized
gatacgggagccaacaccactctccttgaccggggcgaaccagagcaggtgtgacggatg

SEQ ID NO: 137 - DNA, 61 bp, Artificial Sequence
  chemically synthesized
catccgtcacacctgctctggataagatcagcaacaagttagtggtgttggctcccgtatc

SEQ ID NO: 138 - DNA, 60 bp, Artificial Sequence
  chemically synthesized
gatacgggagccaacaccactaacttgttgctgatcttatcagagcaggtgtgacggatg

SEQ ID NO: 139 - DNA, 72 bp, Artificial Sequence
  chemically synthesized
atacgggagccaacaccattaaatcaattgtgccgtgttggtcttgtctcatcgagagcaggtgtgacggat

SEQ ID NO: 140 - DNA, 72 bp, Artificial Sequence
  chemically synthesized
atccgtcacacctgctctcgatgagacaagaccaacacggcacaattgatttaatggtgttggctcccgtat

SEQ ID NO: 141 - DNA, 72 bp, Artificial Sequence
  chemically synthesized
atacgggagccaacaccatttttattatcggtatgatcctacgagttcctcccaagagcaggtgtgacggat

SEQ ID NO: 142 - DNA, 72 bp, Artificial Sequence
  chemically synthesized
atccgtcacacctgctcttgggaggaactcgtcggatcataccgataataaaaatggtgttggctcccgtat

SEQ ID NO: 143 - DNA, 71 bp, Artificial Sequence
  chemically synthesized
atacgggagccaacaccacgtatatcttattatgcacagcatcacgaaagtgcagagcaggtgtgacggat

SEQ ID NO: 144 - DNA, 72 bp, Artificial Sequence
  chemically synthesized
atccgtcacacctgctcttttttattatcggtatgatcctacgagttcctcccatggtgttggctcccgtat

SEQ ID NO: 145 - DNA, 72 bp, Artificial Sequence
  chemically synthesized
atacgggagccaacaccattaacgttaagcggcctcacttcttttaatcctttcagagcaggtgtgacggat

SEQ ID NO: 146 - DNA, 72 bp, Artificial Sequence
  chemically synthesized
atccgtcacacctgctctgaaaggattaaaagaagtgaggccgcttaacgttaatggtgttggctcccgtat

SEQ ID NO: 147 - DNA, 72 bp, Artificial Sequence
  chemically synthesized
atccgtcacacctgctctaatatagaggtattgctcttggacaaggtacagggatggtgttggctcccgtat

SEQ ID NO: 148 - DNA, 72 bp, Artificial Sequence
  chemically synthesized
atacgggagccaacaccatccctgtaccttgtccaagagcaatacctctatattaccacaaccgagggcata

SEQ ID NO: 149 - DNA, 71 bp, Artificial Sequence
  chemically synthesized
atacgggagccaacaccacagtcaagaagttaagagaaaaacaattgtgtataagagcaggtgtgacggat

SEQ ID NO: 150 - DNA, 72 bp, Artificial Sequence
  chemically synthesized
atccgtcacacctgctcttatacacaattgtttttctcttaacttcttgactgctggtgttggctcccgtat

SEQ ID NO: 151 - DNA, 71 bp, Artificial Sequence
  chemically synthesized
atccgtcacacctgctctgcgccacaagattgcggaaagacacccggggggcttggtgttggctcccgtat

SEQ ID NO: 152 - DNA, 70 bp, Artificial Sequence
  chemically synthesized
atacgggagccaacaccagccccccgggtgtctttccgcaatcttgtggcgcagagcaggtgtgacggat

SEQ ID NO: 153 - DNA, 57 bp, Artificial Sequence
  chemically synthesized
atccgtcacacctgctctggccttatgtaaagcgttgggtggtgttggctcccgtat

SEQ ID NO: 154 - DNA, 57 bp, Artificial Sequence
  chemically synthesized
atacgggagccaacaccacccaacgctttacataaggccagagcaggtgtgacggat

SEQ ID NO: 155 - DNA, 72 bp, Artificial Sequence; identical to SEQ ID NO: 19
  chemically synthesized

SEQ ID NO: 156 - DNA, 72 bp, Artificial Sequence
  chemically synthesized
atccgtcactcctgctctgtagatggcaaggcataagcgtccggaacgatagaatggtgttggctcccgtat

SEQ ID NO: 157 - DNA, 72 bp, Artificial Sequence
  chemically synthesized
atacgggagccaacaccatgaatatctcttctacctcctctcctccctttacttagagcaggtgtgacggat

SEQ ID NO: 158 - DNA, 72 bp, Artificial Sequence
  chemically synthesized
atccgtcactcctgctctaagtaaagggaggagaggaggtagaagagatattcatggtgttggctcccgtat

SEQ ID NO: 159 - DNA, 72 bp, Artificial Sequence
  chemically synthesized
atacgggagccaacaccacgccgccccagttcatggcctctatgtccggcaacgagagcaggtgtgacggat

SEQ ID NO: 160 - DNA, 72 bp, Artificial Sequence
  chemically synthesized
atccgtcactcctgctctcgttgccggacatagaggccatgaactggggcggcgtggtgttggctcccgtat

SEQ ID NO: 161 - DNA, 72 bp, Artificial Sequence
  chemically synthesized
atacgggagccaacaccatctagatctgaagaataaaacaaagacaaagatgctagagcaggtgtgacggat

SEQ ID NO: 162 - DNA, 69 bp, Artificial Sequence
  chemically synthesized
atccgtcactcctgctctagcatctttgtctttgttttattcagatctagatggtgttggctcccgtat

SEQ ID NO: 163 - DNA, 72 bp, Artificial Sequence; identical to SEQ ID NO: 14
  chemically synthesized

SEQ ID NO: 164 - DNA, 72 bp, Artificial Sequence
  chemically synthesized
atccgtcactcctgctctggtggaatggactaagctagctagcgttttaaaaggtggtgttggctcccgtat

SEQ ID NO: 165 - DNA, 58 bp, Artificial Sequence
  chemically synthesized
atacgggagccaacaccacgcttagatattatccttgtccagagcaggtgtgacggat

SEQ ID NO: 166 - DNA, 58 bp, Artificial Sequence
  chemically synthesized
atccgtcacacctgctctggacaaggataatatctaagcgtggtgttggctcccgtat

SEQ ID NO: 167 - DNA, 72 bp, Artificial Sequence
  chemically synthesized
acacgggagccaacaccatccatagctcatctataccctcttccgagtcccaccagagcaggtgtgacggat

SEQ ID NO: 168 - DNA, 72 bp, Artificial Sequence
  chemically synthesized
atccgtcacacctgctctggtgggactcggaagagggtatagatgagctatggatggtgttggctcccgtgt

SEQ ID NO: 169 - DNA, 72 bp, Artificial Sequence
  chemically synthesized
atacgggagccaacaccaccctacaccagcgccctacacttttgtagcacttcgagagcaggtgtgacggat

SEQ ID NO: 170 - DNA, 72 bp, Artificial Sequence
  chemically synthesized
atccgtcacacctgctctcgaagtgctacaaaagtgtagggcgctggtgtagggtggtgttggctcccgtat

SEQ ID NO: 171 - DNA, 69 bp, Artificial Sequence
  chemically synthesized
atacgggagccaacaccatagtgttgggccaatacggtaacgtgtccttggagagcaggtgtgacggat

SEQ ID NO: 172 - DNA, 69 bp, Artificial Sequence
  chemically synthesized
atccgtcacacctgctctccaaggacacgttaccgtattggcccaacactatggtgttggctcccgtat

SEQ ID NO: 173 - DNA, 72 bp, Artificial Sequence
  chemically synthesized
atacgggagccaacaccacccgtttttgatctaatgaggatacaatattcgtctagagcaggtgtgacggat

SEQ ID NO: 174 - DNA, 72 bp, Artificial Sequence
  chemically synthesized
atccgtcacacctgctctagacgaatattgtatcctcattagatcaaaaacgggtggtgttggctcccgtat

SEQ ID NO: 175 - DNA, 72 bp, Artificial Sequence
  chemically synthesized
atacgggagccaacaccagcttttcctagaatgattttctttagctacctgagaagagcaggtgtgacggat

SEQ ID NO: 176 - DNA, 72 bp, Artificial Sequence
  chemically synthesized
atccgtcacacctgctcttctcaggtagctaaagaaaatcattctaggaaaagctggtgttggctcccgtat

SEQ ID NO: 177 - DNA, 58 bp, Artificial Sequence
  chemically synthesized
atacgggagccaacaccacgattagcaatgaattatctacagagcaggtgtgacggat

SEQ ID NO: 178 - DNA, 58 bp, Artificial Sequence
  chemically synthesized
atccgtcacacctgctctgtagataattcattgctaatcgtggtgttggctcccgtat

SEQ ID NO: 179 - DNA, 72 bp, Artificial Sequence
  chemically synthesized
atacgggagccaacaccaattctaacacaggtttctccgtttcgttagctgctaagagcaggtgtgacggat

SEQ ID NO: 180 - DNA, 72 bp, Artificial Sequence
  chemically synthesized
atccgtcacacctgctcttagcagctaacgaaacggagaaacctgtgttagaattggtgttggctcccgtat

SEQ ID NO: 181 - DNA, 72 bp, Artificial Sequence; identical to SEQ ID NO: 19
  chemically synthesized

SEQ ID NO: 182 - DNA, 72 bp, Artificial Sequence; identical to SEQ ID NO: 12
  chemically synthesized

SEQ ID NO: 183 - DNA, 72 bp, Artificial Sequence
  chemically synthesized
atacgggagccaacaccaactgactcagtctgctggtgggctatatttttgcggagagcaggtgtgacggat

SEQ ID NO: 184 - DNA, 72 bp, Artificial Sequence
  chemically synthesized
atccgtcacacctgctctccgcaaaaatatagcccaccagcagactgagtcagttggtgttggctcccgtat

SEQ ID NO: 185 - DNA, 72 bp, Artificial Sequence
  chemically synthesized
atacgggagccaacaccatatagccgcgcctgtgagttttgtgggagcaagagtagagcaggtgtgacggat

SEQ ID NO: 186 - DNA, 72 bp, Artificial Sequence
  chemically synthesized
atccgtcacacctgctctactcttgctcccacaaaactcacaggcgcggctatatggtgttggctcccgtat

SEQ ID NO: 187 - DNA, 72 bp, Artificial Sequence
  chemically synthesized
atacgggagccaacaccagctacagtgtcagacggttccaccttaacctcgtcaagagcaggtgtgacggat

SEQ ID NO: 188 - DNA, 72 bp, Artificial Sequence
  chemically synthesized
atccgtcacacctgctcttgacgaggttaaggtggaaccgtctgacactgtagctggtgttggctcccgtat

SEQ ID NO: 189 - DNA, 72 bp, Artificial Sequence
  chemically synthesized
atacgggagccaacaccattgactaagcgattagtcccacaggtgaccggggagagagcaggtgtgacggat

SEQ ID NO: 190 - DNA, 72 bp, Artificial Sequence
  chemically synthesized
atccgtcacacctgctctcttcccggnctcctgtgtgattaatctgttattctatggtgttggctcccgtat

SEQ ID NO: 191 - DNA, 72 bp, Artificial Sequence; identical to SEQ ID NO: 179
  chemically synthesized

SEQ ID NO: 192 - DNA, 72 bp, Artificial Sequence; identical to SEQ ID NO: 180
  chemically synthesized

SEQ ID NO: 193 - DNA, 72 bp, Artificial Sequence
  chemically synthesized
atacgggagccaacaccactcatcccgttggaacactttaatatggcccactctagagcaggtgtgacggat

SEQ ID NO: 194 - DNA, 72 bp, Artificial Sequence
  chemically synthesized
atccgtcacacctgctctagagtgggccatattaaagtgttccaacgggatgagtggtgttggctcccgtat

SEQ ID NO: 195 - DNA, 59 bp, Artificial Sequence
  chemically synthesized
atacgggagccaacaccactaacttgttgctgatcttatccagagcaggtgtgacggat

SEQ ID NO: 196 - DNA, 59 bp, Artificial Sequence
  chemically synthesized
atccgtcacacctgctctggataagatcagcaacaagttagtggtgttggctcccgtat

SEQ ID NO: 197 - DNA, 72 bp, Artificial Sequence; identical to SEQ ID NO: 173
  chemically synthesized

SEQ ID NO: 198 - DNA, 72 bp, Artificial Sequence; identical to SEQ ID NO: 174
  chemically synthesized

SEQ ID NO: 199 - DNA, 71 bp, Artificial Sequence
  chemically synthesized
atacgggagccaacaccaatcgatggttagctattacactagatggaattcatagagcaggtgtgacggat

SEQ ID NO: 200 - DNA, 72 bp, Artificial Sequence
  chemically synthesized
atccgtcacacctgctctatgaattccatctagtgtaatagtctaaccatcgattggtgttggctcccgtat

SEQ ID NO: 201 - DNA, 72 bp, Artificial Sequence
  chemically synthesized
atacgggagccaacaccaatctgccgactaggccaagtaattatattcagctggagagcaggtgtgacggat

SEQ ID NO: 202 - DNA, 72 bp, Artificial Sequence
  chemically synthesized
atccgtcacacctgctctccagctgaatataattacttggcctagtcggcagattggtgttggctcccgtat

SEQ ID NO: 203 - DNA, 59 bp, Artificial Sequence; identical to SEQ ID NO: 16
  chemically synthesized

SEQ ID NO: 204 - DNA, 59 bp, Artificial Sequence; identical to SEQ ID NO: 9
  chemically synthesized

SEQ ID NO: 205 - DNA, 59 bp, Artificial Sequence; identical to SEQ ID NO: 16
  chemically synthesized

SEQ ID NO: 206 - DNA, 59 bp, Artificial Sequence; identical to SEQ ID NO: 9
  chemically synthesized

SEQ ID NO: 207 - DNA, 72 bp, Artificial Sequence
  chemically synthesized
atacgggagccaacaccacattacaatagatgtattgacatatccggacagtcgagagcaggtgtgacggat

SEQ ID NO: 208 - DNA, 72 bp, Artificial Sequence
  chemically synthesized
atccgtcacacctgctctcgactgtccggatatgtcaatacatctattgtaatgtggtgttggctcccgtat

SEQ ID NO: 209 - DNA, 72 bp, Artificial Sequence; identical to SEQ ID NO: 173
  chemically synthesized

SEQ ID NO: 210 - DNA, 72 bp, Artificial Sequence; identical to SEQ ID NO: 174
  chemically synthesized

SEQ ID NO: 211 - DNA, 72 bp, Artificial Sequence
  chemically synthesized
atacgggagccaacaccactcgtgtagtgctgtctttgtggaatccttgcatcgagagcaggtgtgacggat

SEQ ID NO: 212 - DNA, 72 bp, Artificial Sequence
  chemically synthesized
atccgtcacacctgctctcgatgcaaggattccacaaagacagcactacacgagtggtgttggctcccgtat

SEQ ID NO: 213 - DNA, 72 bp, Artificial Sequence
  chemically synthesized
atacgggagccaacaccaccacgtgacccatacgatacaacaaataattgctcaagagcaggtgtgacggat

SEQ ID NO: 214 - DNA, 72 bp, Artificial Sequence
  chemically synthesized
atccgtcacacctgctcttgagcaattatttgttgtatcgtatgggtcacgtggtggtgttggctcccgtat

SEQ ID NO: 215 - DNA, 72 bp, Artificial Sequence
  chemically synthesized
atacgggagccaacaccatccatagctcatctataccctcttccgagtcccaccagagcaggtgtgacggat

SEQ ID NO: 216 - DNA, 72 bp, Artificial Sequence
  chemically synthesized
atccgtcacacctgctctggtgggactcggaagagggtatagatgagctatggatggtgttggctcccgtat

SEQ ID NO: 217 - DNA, 72 bp, Artificial Sequence
  chemically synthesized
atacgggagccaacaccagacgcggaacgactcatcgcaaaatgtcgtgatgcaagagcaggtgtgacggat

SEQ ID NO: 218 - DNA, 72 bp, Artificial Sequence
  chemically synthesized
atccgtcacacctgctcttgcatcacgacattttgcgatgagtcgttccgcgtctggtgttggctcccgtat

SEQ ID NO: 219 - DNA, 72 bp, Artificial Sequence
  chemically synthesized
atacgggagccaacaccatggttaggctgctccatatattcccgccccgcacgtagagcaggtgtgacggat

SEQ ID NO: 220 - DNA, 73 bp, Artificial Sequence
  chemically synthesized
ataccgtcacacctgctctacgtgcggggcgggaatatatggagcagcctaaccatggtgttggctcccgtat

SEQ ID NO: 221 - DNA, 72 bp, Artificial Sequence; identical to SEQ ID NO: 173
  chemically synthesized

SEQ ID NO: 222 - DNA, 72 bp, Artificial Sequence; identical to SEQ ID NO: 174
  chemically synthesized

SEQ ID NO: 223 - DNA, 72 bp, Artificial Sequence
  chemically synthesized
atacgggagccaacaccacccgtttttgatcttatgaggatacaatattcgtctagagcaggtgtgacggat

SEQ ID NO: 224 - DNA, 72 bp, Artificial Sequence
  chemically synthesized
atccgtcacacctgctctagacgaatattgtatcctcataagatcaaaaacgggtggtgttggctcccgtat

SEQ ID NO: 225 - DNA, 72 bp, Artificial Sequence
  chemically synthesized
atccgtcacacctgctctccgaccaatgtgtggatcattactaatcgactattgtggtgttggctcccgtat

SEQ ID NO: 226 - DNA, 70 bp, Artificial Sequence
  chemically synthesized
atacgggagccaacaccacaatagtcgattagtaatgccacacattggtcggagagcaggtgtgacggat

SEQ ID NO: 227 - DNA, 68 bp, Artificial Sequence
  chemically synthesized
atacgggagccaacaccatagttttggccaatacggtaacgtgtccttggagagcaggtgtgacggat

SEQ ID NO: 228 - DNA, 69 bp, Artificial Sequence
  chemically synthesized
atccgtcacacctgctctccaaggacacgttaccgtattggcccaaaactatggtgttggctcccgtat

SEQ ID NO: 229 - DNA, 69 bp, Artificial Sequence
  chemically synthesized
atccgtcacacctgctctccaaggacacgttaccgtattggtccagcactatggtgttggctcccgtat

SEQ ID NO: 230 - DNA, 69 bp, Artificial Sequence
  chemically synthesized
atacgggagccaacaccatagtgctggaccaatacggtaacgtgtccttggagagcaggtgtgacggat

SEQ ID NO: 231 - DNA, 72 bp, Artificial Sequence
  chemically synthesized
atccgtcacacctgctctaacgtgtgggttgaagtgtcgccaacaaattgatagtggtgttggctcccgtat

SEQ ID NO: 232 - DNA, 72 bp, Artificial Sequence
  chemically synthesized
atacgggagccaacaccactatcaatttgttggcgacacttcaacccacacgttagagcaggtgtgacggat

SEQ ID NO: 233 - DNA, 72 bp, Artificial Sequence
  chemically synthesized
atccgtcacacctgctctcgaccaaatatttcccccagctctaacccatgctgatggtgttggctcccgtat

SEQ ID NO: 234 - DNA, 72 bp, Artificial Sequence
  chemically synthesized
atacgggagccaacaccatcagcatgggttagagctgggggaaatatttggtcgagagcaggtgtgacggat

SEQ ID NO: 235 - DNA, 69 bp, Artificial Sequence; identical to SEQ ID NO: 171
  chemically synthesized

SEQ ID NO: 236 - DNA, 69 bp, Artificial Sequence; identical to SEQ ID NO: 172
  chemically synthesized

SEQ ID NO: 237 - DNA, 72 bp, Artificial Sequence
  chemically synthesized
atccgtcacacctgctctggggtccgcttgggaacgatattcctgttgttttgttggtgttggctcccgtat

SEQ ID NO: 238 - DNA, 72 bp, Artificial Sequence
  chemically synthesized
atacgggagccaacaccaacaaaacaacaggaatatcgttcccaagcggaccccagagcaggtgtgacggat

SEQ ID NO: 239 - DNA, 72 bp, Artificial Sequence
  chemically synthesized
atccgtcacacctgctctgatggcaacatgggttaaatctaacaacactttgtatggtgttggctcccgtat

SEQ ID NO: 240 - DNA, 72 bp, Artificial Sequence
  chemically synthesized
atacgggagccaacaccatacaaagtgttgttagatttaacccatgttgccatcagagcaggtgtgacggat

SEQ ID NO: 241 - DNA, 72 bp, Artificial Sequence
  chemically synthesized
atacgggagccaacaccaagggtgttcacactggcaggcgacgccctcgtgttgagagcaggtgtgacggat

SEQ ID NO: 242 - DNA, 68 bp, Artificial Sequence
  chemically synthesized
atccgtcacacctgctctcaacacgagggcgtcgcctgccagtgtgaaattggtgttggctcccgtat

SEQ ID NO: 243 - DNA, 72 bp, Artificial Sequence
  chemically synthesized
atacgggagccaacaccatagacaatggcgtacttttcgtaattccacaagaatagagcaggtgtgacggat

SEQ ID NO: 244 - DNA, 72 bp, Artificial Sequence
  chemically synthesized
atccgtcacacctgctctattcttgtggaattacgaaaagtacgccattgtctatggtgttggctcccgtat

SEQ ID NO: 245 - DNA, 58 bp, Artificial Sequence
  chemically synthesized
atacgggagccaacaccaccacaaaagcattcgcccttacagagcaggtgtgacggat

SEQ ID NO: 246 - DNA, 58 bp, Artificial Sequence
  chemically synthesized
atccgtcacacctgctctgtaagggcgaatgcttttgtggtggtgttggctcccgtat

SEQ ID NO: 247 - DNA, 72 bp, Artificial Sequence
  chemically synthesized
atacgggagccaacaccagcgtgtagctagtttcaggattgtagtatgtaatatagagcaggtgtgacggat

SEQ ID NO: 248 - DNA, 72 bp, Artificial Sequence
  chemically synthesized
atccgtcacacctgctctatattacatactacaatcctgaaactagctacacgctggtgttggctcccgtat

SEQ ID NO: 249 - DNA, 58 bp, Artificial Sequence
  chemically synthesized
atacgggagccaacaccacgcacatactagctatctcatcagagcaggtgtgacggat

SEQ ID NO: 250 - DNA, 58 bp, Artificial Sequence
  chemically synthesized
atccgtcacacctgctctgatgagatagctagtatgtgcgtggtgttggctcccgtat

SEQ ID NO: 251 - DNA, 72 bp, Artificial Sequence
  chemically synthesized
atacgggagccaacaccatcagagatcatctaacgaaaatcatgggtctcgcccagagcaggtgtgacggat

SEQ ID NO: 252 - DNA, 72 bp, Artificial Sequence
  chemically synthesized
atccgtcacacctgctctgggcgagacccatgattttcgttagatgatctctgatggtgttggctcccgtat

SEQ ID NO: 253 - DNA, 72 bp, Artificial Sequence
  chemically synthesized
atacgggagccaacaccagcaaagaatagtgagccctatgatcatctgttcgtcagagcaggtgtgacggat

SEQ ID NO: 254 - DNA, 72 bp, Artificial Sequence
  chemically synthesized
atccgtcacacctgctctgacgaacagatgatcatagggctcactattctttgctggtgttggctcccgtat

SEQ ID NO: 255 - DNA, 72 bp, Artificial Sequence
  chemically synthesized
atacgggagccaacaccagacatcatgtcgcatatctggatctagaggctattcagagcaggtgtgacggat

SEQ ID NO: 256 - DNA, 73 bp, Artificial Sequence
  chemically synthesized
ataccgtcacacctgctctgaatagcctctagatccagatatgcgacatgatgtctggtgttggctcccgtat

SEQ ID NO: 257 - DNA, 72 bp, Artificial Sequence
  chemically synthesized
atacgggagccaacaccagtacggcggtgtccgaactcactatacccagttgaaagagcaggtgtgacggat

SEQ ID NO: 258 - DNA, 72 bp, Artificial Sequence
  chemically synthesized
atccgtcacacctgctctttcaactgggtatagtgagttcggacaccgccgtactggtgttggctcccgtat

SEQ ID NO: 259 - DNA, 71 bp, Artificial Sequence
  chemically synthesized
atacgggagccaacaccagacctgacaacgaaaccccagttgtcgccatagccagagcaggtgtgacggat

SEQ ID NO: 260 - DNA, 72 bp, Artificial Sequence
  chemically synthesized
atccgtcacacctgctctggctatggcgacaactggggttttcgttgtcaggtctggtgttggctcccgtat

SEQ ID NO: 261 - DNA, 69 bp, Artificial Sequence; identical to SEQ ID NO: 171
  chemically synthesized

SEQ ID NO: 262 - DNA, 69 bp, Artificial Sequence; identical to SEQ ID NO: 172
  chemically synthesized

SEQ ID NO: 263 - DNA, 72 bp, Artificial Sequence
  chemically synthesized
atacgggagccaacaccataagcgcaacacagtccatccctgagtgagatagcgagagcaggtgtgacggat

SEQ ID NO: 264 - DNA, 73 bp, Artificial Sequence
  chemically synthesized
atccgtcacaccctgctctcgctatctcactcagggatggactgtgttgcgcttatggtgttggctcccgtat

SEQ ID NO: 265 - DNA, 58 bp, Artificial Sequence; identical to SEQ ID NO: 249
  chemically synthesized

SEQ ID NO: 266 - DNA, 58 bp, Artificial Sequence; identical to SEQ ID NO: 250
  chemically synthesized

SEQ ID NO: 267 - DNA, 58 bp, Artificial Sequence
  chemically synthesized
atacgggagccaacaccactaacttgttgctgatcttaccagagcaggtgtgacggat

SEQ ID NO: 268 - DNA, 58 bp, Artificial Sequence
  chemically synthesized
atccgtcacacctgctctggtaagatcagcaacaagttagtggtgttggctcccgtat

SEQ ID NO: 269 - DNA, 72 bp, Artificial Sequence
  chemically synthesized
atacgggagccaacaccacccgtttttgatctaatgaggatacaatattcgtcnagagcaggtgtgacggat

SEQ ID NO: 270 - DNA, 73 bp, Artificial Sequence
  chemically synthesized
ataccgtcacacctgctctngacgaatattgtatcctcattagatcaaaaacgggtggtgttggctcccgtat

SEQ ID NO: 271 - DNA, 72 bp, Artificial Sequence
  chemically synthesized
atacgggagccaacaccagttgtgggaacatcaggctaagtatgagacggaacgagagcaggtgtgacggat

SEQ ID NO: 272 - DNA, 72 bp, Artificial Sequence
  chemically synthesized
atccgtcacacctgctctcgttccgtctcatacttagcctgatgttcccacaactggtgttggctcccgtat

SEQ ID NO: 273 - DNA, 69 bp, Artificial Sequence; identical to SEQ ID NO: 171
  chemically synthesized

SEQ ID NO: 274 - DNA, 69 bp, Artificial Sequence; identical to SEQ ID NO: 172
  chemically synthesized

SEQ ID NO: 275 - DNA, 60 bp, Artificial Sequence
  chemically synthesized
catccgtcacacctgctctggggatgtgtggtgttggctcccgtatcaagggcgaattct

SEQ ID NO: 276 - DNA, 60 bp, Artificial Sequence
  chemically synthesized
gtaggcagtgtggacgagacccctacacaccacaaccgagggcatagttcccgcttaaga

SEQ ID NO: 277 - DNA, 60 bp, Artificial Sequence
  chemically synthesized
catccgtcacacctgctctgctatcacatgcctgctgaagtggtgttggctcccgtatca

SEQ ID NO: 278 - DNA, 60 bp, Artificial Sequence
  chemically synthesized
gtaggcagtgtggacgagacgatagtgtacggacgacttcaccacaaccgagggcatagt

SEQ ID NO: 279 - DNA, 72 bp, Artificial Sequence
  chemically synthesized
atccgtcacacctgctctcgatgtctgggccctaatattggtttgcttgtaccatggtgttggctcccgtat

SEQ ID NO: 280 - DNA, 72 bp, Artificial Sequence
  chemically synthesized
atacgggagccaacaccatggtacaagcaaaccaatattagggcccagacatcgagagcaggtgtgacggat

SEQ ID NO: 281 - DNA, 72 bp, Artificial Sequence
  chemically synthesized
atacgggagccaacaccatgataccctaaggtaggggaggcctaagcgccacgtagagcaggtgtgacggat

SEQ ID NO: 282 - DNA, 72 bp, Artificial Sequence
  chemically synthesized
atccgtcacacctgctctacgtggcgcttaggcctcccctaccttagggtatcatggtgttggctcccgtat

SEQ ID NO: 283 - DNA, 72 bp, Artificial Sequence
  chemically synthesized
atacgggagccaacaccagcgatcccccgccgggcccgcgccccgctcgcagacagagcaggtgtgacggat

SEQ ID NO: 284 - DNA, 71 bp, Artificial Sequence
  chemically synthesized
atccgtcacacctgctctgtctgcgagcggggcgcgggcccggcgggggatgcgtggtgttgctcccgtat

SEQ ID NO: 285 - DNA, 73 bp, Artificial Sequence
  chemically synthesized
atccgtcacacctgctctacggcgctcccaacaggcctctccttacggcatattatggtgttggctcccgtat

SEQ ID NO: 286 - DNA, 73 bp, Artificial Sequence
  chemically synthesized
atacgggagccaacaccataatatgccgtaaggagaggcctgttgggagcgccgtagagcaggtgtgacggat

SEQ ID NO: 287 - DNA, 72 bp, Artificial Sequence
  chemically synthesized
atacgggagccaacaccaggaaaaaaagagcctgtgaagattgtaatatcagttagagcaggtgtgacggat

SEQ ID NO: 288 - DNA, 72 bp, Artificial Sequence
  chemically synthesized
atccgtcacacctgctctaactgatattacaatcttcacaggctctttttttcctggtgttggctcccgtat

SEQ ID NO: 289 - DNA, 72 bp, Artificial Sequence
  chemically synthesized
atccgtcacacctgctctcggaggtagactaggattgcggcggggggtcaggtatggtgttggctcccgtat

SEQ ID NO: 290 - DNA, 71 bp, Artificial Sequence
  chemically synthesized
atacgggagccaacaccacaaaagccttacctaactgccaacaatgaatagcaagagcaggtgtgacggat

SEQ ID NO: 291 - DNA, 72 bp, Artificial Sequence
  chemically synthesized
atccgtcacacctgctcttgctattcattgttggcagttaggtaaggcttttgttggtgttggctcccgtat

SEQ ID NO: 292 - DNA, 72 bp, Artificial Sequence
  chemically synthesized
atacgggagccaacaccatacctgaccccccgccgcaatcctagtctacctccgagagcaggtgtgacggat

SEQ ID NO: 293 - DNA, 72 bp, Artificial Sequence
  chemically synthesized
atacgggagccaacaccacgactaacacgaccgttggggggggctcgcgcgggcagagcaggtgtgacggat

SEQ ID NO: 294 - DNA, 72 bp, Artificial Sequence
  chemically synthesized
atccgtcacacctgctctgcccgcgcgagccccccccaacggtcgtgttagtcgtggtgttggctcccgtat

SEQ ID NO: 295 - DNA, 72 bp, Artificial Sequence
  chemically synthesized
atacgggagccaacaccagtccccgcccagccgtgagccgtacccccgcacaccagagcaggtgtgacggat

SEQ ID NO: 296 - DNA, 72 bp, Artificial Sequence
  chemically synthesized
atccgtcacacctgctctggtgtgcgggggtacggctcacggctgggcggggactggtgttggctcccgtat

SEQ ID NO: 297 - DNA, 72 bp, Artificial Sequence
  chemically synthesized
atccgtcacacctgctctcaaggttgggcctgcaagagcaaaaacggggcgggatggtgttggctcccgtat

SEQ ID NO: 298 - DNA, 71 bp, Artificial Sequence
  chemically synthesized
atacgggagccaacacatcccgccccgtttttgctcttgcaggcccaaccttgagagcaggtgtgacggat

SEQ ID NO: 299 - DNA, 72 bp, Artificial Sequence
  chemically synthesized
atccgtcacacctgctctacttggcttgcgactattattcacagggccaaagactggtgttggctcccgtat

SEQ ID NO: 300 - DNA, 72 bp, Artificial Sequence
  chemically synthesized
atacgggagccaacaccagtctttggccctgtgaataatagtcgcaagccaagtagagcaggtgtgacggat

SEQ ID NO: 301 - DNA, 69 bp, Artificial Sequence
  chemically synthesized
atacgggagccaacaccatagtgttggaccaatacggtaacgtgtccttggagagcaggtgtgacggat

SEQ ID NO: 302 - DNA, 69 bp, Artificial Sequence
  chemically synthesized
atccgtcacacctgctctccaaggacacgttaccgtattggtccaacactatggtgttggctcccgtat

SEQ ID NO: 303 - DNA, 72 bp, Artificial Sequence
  chemically synthesized
atccgtcacacctgctcttggaatgtcggtgtttttccaattccttgggtcgtgtggtgttggctcccgtat

SEQ ID NO: 304 - DNA, 72 bp, Artificial Sequence
  chemically synthesized
atacgggagccaacaccacacgacccaaggaattggaaaaacaccgacattccaagagcaggtgtgacggat

SEQ ID NO: 305 - DNA, 72 bp, Artificial Sequence
  chemically synthesized
atccgtcacacctgctctgcgacggcgacgcggtccgggcgggggtggaggacgtggtgttggctcccgtat

SEQ ID NO: 306 - DNA, 72 bp, Artificial Sequence
  chemically synthesized
atacgggagccaacaccacgtcctccacccccgcccggaccgcgtcgccgtcgcagagcaggtgtgacggat

SEQ ID NO: 307 - DNA, 72 bp, Artificial Sequence
  chemically synthesized
atacgggagccaacaccagagggttctagggtcacttccatgagaatggctcacagagcaggtgtgacggat

SEQ ID NO: 308 - DNA, 72 bp, Artificial Sequence
  chemically synthesized
atccgtcacacctgctctggcctggggacgcgagggaggcggggggagtcgtggtggtgttggctcccgtat

SEQ ID NO: 309 - DNA, 72 bp, Artificial Sequence
  chemically synthesized
atacgggagccaacaccaccacgactccccccgcctccctcgcgtccccaggccagagcaggtgtgacggat

SEQ ID NO: 310 - DNA, 71 bp, Artificial Sequence
  chemically synthesized
atccgtcacacctgctctgtgagccattgtcatggaatgaccctagaaccctctggtgttggctcccgtat

SEQ ID NO: 311 - DNA, 72 bp, Artificial Sequence
  chemically synthesized
atccgtcacacctgctctcacagggcctcttactatacagttctccagcgctgctggtgttggctcccgtat

SEQ ID NO: 312 - DNA, 72 bp, Artificial Sequence
  chemically synthesized
atacgggagccaacaccagcagcgctggagaactgtatagtaagaggccctgtgagagcaggtgtgacggat

SEQ ID NO: 313 - DNA, 72 bp, Artificial Sequence
  chemically synthesized
atccgtcacacctgctctgcacgggctcagtttggctttgtatcctaagagagatggtgttggctcccgtat

SEQ ID NO: 314 - DNA, 72 bp, Artificial Sequence
  chemically synthesized
atacgggagccaacaccatctctcttaggatacaaagccaaactgagcccgtgcagagcaggtgtgacggat

SEQ ID NO: 315 - DNA, 71 bp, Artificial Sequence
  chemically synthesized
atacgggagccaacaccaggggtggcgaacatggtataacttgataagtgtgaagagcaggtgtgacggat

SEQ ID NO: 316 - DNA, 72 bp, Artificial Sequence
  chemically synthesized
atccgtcacacctgctcttcacacttatcaagttataccatgttcgccaccccctggtgttggctcccgtat

SEQ ID NO: 317 - DNA, 72 bp, Artificial Sequence
  chemically synthesized
atacgggagccaacaccactccgacaccggccgccggcaccacccactccccctagagcaggtgtgacggat

SEQ ID NO: 318 - DNA, 72 bp, Artificial Sequence
  chemically synthesized
atccgtcacacctgctctagggggagtgggtggtgccggcggccggtgtcggagtggtgttggctcccgtat

SEQ ID NO: 319 - DNA, 72 bp, Artificial Sequence
  chemically synthesized
atacgggagccaacaccatccggcgcgccctcctcccccactgctccccgcccgagagcaggtgtgacggat

SEQ ID NO: 320 - DNA, 72 bp, Artificial Sequence
  chemically synthesized
atccgtcacacctgctctcgggcggggagcagtgggggaggagggcgcgccggatggtgttggctcccgtat

SEQ ID NO: 321 - DNA, 72 bp, Artificial Sequence
  chemically synthesized
atacgggagccaacaccatacgcagaggtcccctacccaggccagccggatgccagagcaggtgtgacggat

SEQ ID NO: 322 - DNA, 72 bp, Artificial Sequence
  chemically synthesized
atccgtcacacctgctctggcatccggctggcctgggtaggggacctctgcgtatggtgttggctcccgtat

SEQ ID NO: 323 - DNA, 71 bp, Artificial Sequence
  chemically synthesized
atccgtccacctgctctggagacattaaaaaccggagtttatttatacctttctggtgttggctcccgtat

SEQ ID NO: 324 - DNA, 72 bp, Artificial Sequence
  chemically synthesized
atacgggagccaacaccagaaaggtataaataaactccggtttttaatgtctccagagcaggtgtgacggat

SEQ ID NO: 325 - DNA, 59 bp, Artificial Sequence; identical to SEQ ID NO: 195
  chemically synthesized

SEQ ID NO: 326 - DNA, 59 bp, Artificial Sequence; identical to SEQ ID NO: 196
  chemically synthesized

SEQ ID NO: 327 - DNA, 58 bp, Artificial Sequence
  chemically synthesized
atccgtcacacctgctctgcatggagagttttttggtcagtggtgttggctcccgtat

SEQ ID NO: 328 - DNA, 58 bp, Artificial Sequence
  chemically synthesized
atacgggagccaacaccactgaccaaaaaactctccatgcagagcaggtgtgacggat

SEQ ID NO: 329 - DNA, 57 bp, Artificial Sequence
  chemically synthesized
atagggagccaacaccacgttaacgcgtagcctttggacagagcaggtgtgacggat

SEQ ID NO: 330 - DNA, 58 bp, Artificial Sequence; identical to SEQ ID NO: 53
  chemically synthesized

SEQ ID NO: 331 - DNA, 59 bp, Artificial Sequence; identical to SEQ ID NO: 9
  chemically synthesized

SEQ ID NO: 332 - DNA, 59 bp, Artificial Sequence; identical to SEQ ID NO: 16
  chemically synthesized

SEQ ID NO: 333 - DNA, 72 bp, Artificial Sequence
  chemically synthesized
atccgtcacacctgctctcgtccgtcattaagttcggaggctggcgggttgcgttggtgttggctcccgtat

SEQ ID NO: 334 - DNA, 72 bp, Artificial Sequence
  chemically synthesized
atacgggagccaacaccaacgcaacccgccagcctccgaacttaatgacggacgagagcaggtgtgacggat

SEQ ID NO: 335 - DNA, 72 bp, Artificial Sequence; identical to SEQ ID NO: 19
  chemically synthesized

SEQ ID NO: 336 - DNA, 73 bp, Artificial Sequence
  chemically synthesized
atccgtcacacctgctctgtagatggcaaggcataagcgtccggaaacgatagaatggtgttggctcccgtat

SEQ ID NO: 337 - DNA, 71 bp, Artificial Sequence
  chemically synthesized
tccgtcacacctgctctaactcttactactttgttgctatcacattcaactgttggtgttggctcccgtat

SEQ ID NO: 338 - DNA, 72 bp, Artificial Sequence
  chemically synthesized
atacgggagccaacaccaacagttgaatgtgatagcaacaaagtagtaagagttagagcaggtgtgacggat

SEQ ID NO: 339 - DNA, 58 bp, Artificial Sequence
  chemically synthesized
atccgtcacacctgctctggcctttcaccaagcgtccttgtggtgttggctcccgtat

SEQ ID NO: 340 - DNA, 58 bp, Artificial Sequence
  chemically synthesized
atacgggagccaacaccacaaggacgcttggtgaaaggccagagcaggtgtgacggat

SEQ ID NO: 341 - DNA, 58 bp, Artificial Sequence
  chemically synthesized
atccgtcacacctgctctggcaccgagcacgggaacccagtggtgttggctcccgtat

SEQ ID NO: 342 - DNA, 58 bp, Artificial Sequence
  chemically synthesized
atacgggagccaacaccactgggttcccgtgctcggtgccagagcaggtgtgacggat

SEQ ID NO: 343 - DNA, 69 bp, Artificial Sequence; identical to SEQ ID NO: 171
  chemically synthesized

SEQ ID NO: 344 - DNA, 69 bp, Artificial Sequence; identical to SEQ ID NO: 172
  chemically synthesized

SEQ ID NO: 345 - DNA, 72 bp, Artificial Sequence
  chemically synthesized
atccgtcacacctgctctacccgatgccgccccgggattgttgtatgaccatcttggtgttggctcccgtat

SEQ ID NO: 346 - DNA, 72 bp, Artificial Sequence
  chemically synthesized
atacgggagccaacaccaagatggtcatacaacaatcccggggcggcatcgggtagagcaggtgtgacggat

SEQ ID NO: 347 - DNA, 72 bp, Artificial Sequence
  chemically synthesized
atacgggagccaacaccaccccatgagtacacgtgaacggacacagcctccggcagagcaggtgtgacggat

SEQ ID NO: 348 - DNA, 72 bp, Artificial Sequence
  chemically synthesized
atccgtcacacctgctctgccggaggctgtgtccgttcacgtgtactcatggggtggtgttggctcccgtat

SEQ ID NO: 349 - DNA, 72 bp, Artificial Sequence
  chemically synthesized
atccgtcacacctgctcttaaccattcatttcttttgtggtatgaccgttcgcctggtgttggctcccgtat

SEQ ID NO: 350 - DNA, 72 bp, Artificial Sequence
  chemically synthesized
atacgggagccaacaccaggcgaacggtcataccacaaaagaaatgaatggttaagagcaggtgtgacggat

SEQ ID NO: 351 - DNA, 58 bp, Artificial Sequence
  chemically synthesized
atccgtcacacctgctctggggctcttttcgttaaccaggtggtgttggctcccgtat

SEQ ID NO: 352 - DNA, 58 bp, Artificial Sequence
  chemically synthesized
atacgggagccaacaccacctggttaacgaaaagagccccagagcaggtgtgacggat

SEQ ID NO: 353 - DNA, 72 bp, Artificial Sequence
  chemically synthesized
atacgggagccaacaccagataaattttgcgttcattcttatttcctgtccgccagagcaggtgtgacggat

SEQ ID NO: 354 - DNA, 72 bp, Artificial Sequence; identical to SEQ ID NO: 57
  chemically synthesized

SEQ ID NO: 355 - DNA, 71 bp, Artificial Sequence
  chemically synthesized
atacgggagccaacaccagataaattttggttcattcttatttcctgtccgccagagcaggtgtgacggat

SEQ ID NO: 356 - DNA, 71 bp, Artificial Sequence
  chemically synthesized
atccgtcacacctgctctggcggacaggaaataagaatgaaccaaaatttatctggtgttggctcccgtat

SEQ ID NO: 357 - DNA, 72 bp, Artificial Sequence
  chemically synthesized
atacgggagccaacaccacggggctaccagcaccgtcacccctgattctgccacagagcaggtgtgacggat

SEQ ID NO: 358 - DNA, 72 bp, Artificial Sequence
  chemically synthesized
atccgtcacacctgctctgtggcagaatgaggggtgacggtgctggtagccccgtggtgttggctcccgtat

SEQ ID NO: 359 - DNA, 69 bp, Artificial Sequence
  chemically synthesized
atacgggagccaacaccaaaagttggaaaacactggaaggaaaatgcggtcggagcaggtgtgacggat

SEQ ID NO: 360 - DNA, 69 bp, Artificial Sequence
  chemically synthesized
atccgtcacacctgctctgaccgcattttccttccagtgttttccatcttttggtgttggctcccgtat

SEQ ID NO: 361 - DNA, 72 bp, Artificial Sequence
  chemically synthesized
atacgggagccaacaccaccgggccgatgggcaccaggaactctcggacgagtgagagcaggtgtgacggat

SEQ ID NO: 362 - DNA, 72 bp, Artificial Sequence
  chemically synthesized
atccgtcacacctgctctcactcgtccgagagttcctggtgcccatcggcccggtggtgttggctcccgtat

SEQ ID NO: 363 - DNA, 59 bp, Artificial Sequence; identical to SEQ ID NO: 16
  chemically synthesized

SEQ ID NO: 364 - DNA, 60 bp, Artificial Sequence
  chemically synthesized
atccgtcacacctgtctctgccggaccatccaatatcagctgtggtgttggctcccgtat

SEQ ID NO: 365 - DNA, 72 bp, Artificial Sequence
  chemically synthesized
atacgggagccaacaccagtcgaaaggcggccgtccagtcgagtgatttgacctagagcaggtgtgacggat

SEQ ID NO: 366 - DNA, 72 bp, Artificial Sequence
  chemically synthesized
atccgtcacacctgctctaggtcaaatcactcgactggacggccgcctttcgactggtgttggctcccgtat

SEQ ID NO: 367 - DNA, 72 bp, Artificial Sequence
  chemically synthesized
atacgggagccaacaccacggggcgtgccgtcaaaagaccgagatgtggctgcgagagcaggtgtgacggat

SEQ ID NO: 368 - DNA, 72 bp, Artificial Sequence
  chemically synthesized
atccgtcacacctgctctcgcagccacatctcggtcttttgacggcacgccccgtggtgttggctcccgtat

SEQ ID NO: 369 - DNA, 59 bp, Artificial Sequence; identical to SEQ ID NO: 195
  chemically synthesized

SEQ ID NO: 370 - DNA, 59 bp, Artificial Sequence; identical to SEQ ID NO: 196
  chemically synthesized

SEQ ID NO: 371 - DNA, 72 bp, Artificial Sequence
  chemically synthesized
atacgggagccaacaccatttagcgtagggctcgcttatcatttctcattccctagagcaggtgtgacggat

SEQ ID NO: 372 - DNA, 72 bp, Artificial Sequence
  chemically synthesized
atccgtcacacctgctctagggaatgagaaatgataagcgagccctacgctaaatggtgttggctcccgtat

SEQ ID NO: 373 - DNA, 72 bp, Artificial Sequence
  chemically synthesized
atacgggagccaacaccaccgcaacccaaatctctacacggattatcgtcgagcagagcaggtgtgacggat

SEQ ID NO: 374 - DNA, 72 bp, Artificial Sequence
  chemically synthesized
atccgtcacacctgctctgctcgacgataatccgtgtagagatttgggttgcggtggtgttggctcccgtat

SEQ ID NO: 375 - DNA, 72 bp, Artificial Sequence; identical to SEQ ID NO: 66
  chemically synthesized

SEQ ID NO: 376 - DNA, 72 bp, Artificial Sequence; identical to SEQ ID NO: 58
  chemically synthesized

SEQ ID NO: 377 - DNA, 72 bp, Artificial Sequence
  chemically synthesized
atacgggagccaacaccagcctaccccccctgtacgagggccgcaaccacgtagagagcaggtgtgacggat

SEQ ID NO: 378 - DNA, 72 bp, Artificial Sequence
  chemically synthesized
atccgtcacacctgctctctacgtggttgcggccctcgtacagggggggtaggctggtgttggctcccgtat

SEQ ID NO: 379 - DNA, 59 bp, Artificial Sequence
  chemically synthesized
atacgggagccaacaccacatctagcacgagaccctatcccagagcaggtgtgacggat

SEQ ID NO: 380 - DNA, 59 bp, Artificial Sequence
  chemically synthesized
atccgtcacacctgctctgggatagggtctcgtgctagatgtggtgttggctcccgtat

SEQ ID NO: 381 - DNA, 72 bp, Artificial Sequence
  chemically synthesized
atacgggagccaacaccaacagcgactcgagtctgacgactcgcggggcaaatgagagcaggtgtgacggat

SEQ ID NO: 382 - DNA, 72 bp, Artificial Sequence
  chemically synthesized
atccgtcacacctgctctcatttgccccgcgagtcgtcagactcgagtcgctgttggtgttggctcccgtat

SEQ ID NO: 383 - DNA, 69 bp, Artificial Sequence; identical to SEQ ID NO: 171
  chemically synthesized

SEQ ID NO: 384 - DNA, 69 bp, Artificial Sequence; identical to SEQ ID NO: 172
  chemically synthesized

SEQ ID NO: 385 - DNA, 72 bp, Artificial Sequence
  chemically synthesized
atacgggagccaacaccactaaggagaggtcgcgacagactcttctggtcaaggagagcaggtgtgacggat

SEQ ID NO: 386 - DNA, 73 bp, Artificial Sequence
  chemically synthesized
atccgtcacacctgctctccttgaccagaagagtctgtcgcgacctctccttagtggtgttggctcccgtatg

SEQ ID NO: 387 - DNA, 72 bp, Artificial Sequence
  chemically synthesized
atcgggagccaaacaccaacttcgactcaaagaagtccacgtgagactggtggaagagcaggtgtgacggat

SEQ ID NO: 388 - DNA, 72 bp, Artificial Sequence
  chemically synthesized
atccgtcacacctgctcttccaccagtctcacgtggacttctttgagtcgaagttggtgttggctcccgtat

SEQ ID NO: 389 - DNA, 72 bp, Artificial Sequence
  chemically synthesized
atacgggagccaacaccacccggggagacccgcacgggcgcacaatccttgtctagagcaggtgtgacggat

SEQ ID NO: 390 - DNA, 73 bp, Artificial Sequence
  chemically synthesized
ataccgtcacacctgctctcgacaaggattgtgcgcccgtgcgggtctccccgggtggtgttggctcccgtat

SEQ ID NO: 391 - DNA, 72 bp, Artificial Sequence
  chemically synthesized
atacgggagccaacaccagctggaccaaactacgcccattgtgggggtccccggagagcaggtgtgacggat

SEQ ID NO: 392 - DNA, 72 bp, Artificial Sequence
  chemically synthesized
atccgtcacacctgctctccggggacccccacaatgggcgtagtttggtccagctggtgttggctcccgtat

SEQ ID NO: 393 - DNA, 72 bp, Artificial Sequence
  chemically synthesized
atacgggagccaacaccacgatacccgcttatgaattttaaattaattgtgatcagagcaggtgtgacggat

SEQ ID NO: 394 - DNA, 72 bp, Artificial Sequence
  chemically synthesized
atccgtcacacctgctctgatcacaattaatttaaaattcataagcgggtatcgtggtgttggctcccgtat

SEQ ID NO: 395 - DNA, 72 bp, Artificial Sequence
  chemically synthesized
atacgggagccaacaccaactttcacacatacttgttataccacacgatcttttagagcaggtgtgacggat

SEQ ID NO: 396 - DNA, 72 bp, Artificial Sequence
  chemically synthesized
atccgtcacacctgctctaaaagatcgtgtggtataacaagtatgtgtgaaagttggtgttggctcccgtat

SEQ ID NO: 397 - DNA, 72 bp, Artificial Sequence
  chemically synthesized
atacgggagccaacaccactttgtaactcatttgtagtttgggttgctccccctagagcaggtgtgacggat

SEQ ID NO: 398 - DNA, 72 bp, Artificial Sequence
  chemically synthesized
atccgtcacacctgctctagggggagcaacccaaactacaaatgagttacaaagtggtgttggctcccgtat

SEQ ID NO: 399 - DNA, 72 bp, Artificial Sequence
  chemically synthesized
atacgggagccaacaccatttcccgcttatcttcatccactgcttagcatatgtagagcaggtgtgacggat

SEQ ID NO: 400 - DNA, 72 bp, Artificial Sequence
  chemically synthesized
atccgtcacacctgctctacatatgctaagcagtggatgaagataagcgggaaatggtgttggctcccgtat

SEQ ID NO: 401 - DNA, 72 bp, Artificial Sequence
  chemically synthesized
atacgggagccaacaccaggcactgtatcacaccgtcaagaatgtgatcccctgagagcaggtgtgacggat

SEQ ID NO: 402 - DNA, 72 bp, Artificial Sequence
  chemically synthesized
atccgtcacacctgctctcaggggatcacattcttgacggtgtgatacagtgcctggtgttggctcccgtat

SEQ ID NO: 403 - DNA, 72 bp, Artificial Sequence
  chemically synthesized
atacgggagccaacaccatgtcatttaccttcatcatgacagtgttagtatacgagagcaggtgtgacggat

SEQ ID NO: 404 - DNA, 72 bp, Artificial Sequence
  chemically synthesized
atccgtcacacctgctctaggggatcaaagctatgcgaccatgcgagtggatactggtgttggctcccgtat

SEQ ID NO: 405 - DNA, 72 bp, Artificial Sequence
  chemically synthesized
atacgggagccaacaccagttgccgcctaccttgattattctacattacccgttagagcaggtgtgacggat

SEQ ID NO: 406 - DNA, 72 bp, Artificial Sequence
  chemically synthesized
atccgtcacacctgctctaacgggtaatgtagaataatcaaggtaggcggcaactggtgttggctcccgtat

SEQ ID NO: 407 - DNA, 72 bp, Artificial Sequence
  chemically synthesized
atacgggagccaacaccagtatacatacgaagagttgaaaccaatgcttcgttcagagcaggtgtgacggat

SEQ ID NO: 408 - DNA, 72 bp, Artificial Sequence
  chemically synthesized
atccgtcacacctgctctgaacgaagcattggtttcaactcttcgtatgtatactggtgttggctcccgtat

SEQ ID NO: 409 - DNA, 72 bp, Artificial Sequence
  chemically synthesized
atacgggagccaacaccataccccgaatggctgttttcagtaccaatatgactcagagcaggtgtgacggat

SEQ ID NO: 410 - DNA, 72 bp, Artificial Sequence
  chemically synthesized
atccgtcacacctgctctgagtcatattggtactgaaaacagccattcggggtatggtgttggctcccgtat

SEQ ID NO: 411 - DNA, 72 bp, Artificial Sequence
  chemically synthesized
atacgggagccaacaccactgtcacgatcgtcgttccttttaatccttgtgtctagagcaggtgtgacggat

SEQ ID NO: 412 - DNA, 73 bp, Artificial Sequence
  chemically synthesized
atccgtcacacctgctctagaacacaaggattaaaaggaacgacgatcgtgacagtggtgttggctcccgtat

SEQ ID NO: 413 - DNA, 72 bp, Artificial Sequence
  chemically synthesized
atacgggagccaacaccactggacactgaccctcgcactagctttctgacgggtagagcaggtgtgacggat

SEQ ID NO: 414 - DNA, 72 bp, Artificial Sequence
  chemically synthesized
atccgtcacacctgctctacccggccgaagaatagtgctcggtacttagtcgcgtggtgttggctcccgtat

SEQ ID NO: 415 - DNA, 72 bp, Artificial Sequence
  chemically synthesized
atacgggagccaacaccatttggactttaaatagtggactccttctttgtctcgagagcaggtgtgacggat

SEQ ID NO: 416 - DNA, 72 bp, Artificial Sequence
  chemically synthesized
atccgtcacacctgctctcgagacaaagaaggagtccactatttaaagtccaaatggtgttggctcccgtat

SEQ ID NO: 417 - DNA, 72 bp, Artificial Sequence
  chemically synthesized
atacgggagccaacaccatcctaactggtctaatttttgctgttaccgatcccgagagcaggtgtgacggat

SEQ ID NO: 418 - DNA, 72 bp, Artificial Sequence
  chemically synthesized
atccgtcacacctgctctcgggatcggtaacagcaaaaattagaccagttaggatggtgttggctcccgtat

SEQ ID NO: 419 - DNA, 67 bp, Artificial Sequence
  chemically synthesized
atacgggagccaacacctgacgaaaaaattttatcactaagtgatacgcaagacaggtgtgacggat

SEQ ID NO: 420 - DNA, 72 bp, Artificial Sequence
  chemically synthesized
atccgtcacacctgctcttgcgtatcacttagtgataaaattttttcgtcaggctggtgttggctcccgtat

SEQ ID NO: 421 - DNA, 71 bp, Artificial Sequence
  chemically synthesized
atacgggagccaacaccagacctacttcagaaacggaaatgttcttagccgtcagagcaggtgtgacggat

SEQ ID NO: 422 - DNA, 71 bp, Artificial Sequence
  chemically synthesized
atccgtcacacctgctctgacggctaagaacatttccgtttctgaagtaggtctggtgttggctcccgtat

SEQ ID NO: 423 - DNA, 72 bp, Artificial Sequence
  chemically synthesized
atacgggagccaacaccaggccaacgaaactcctactacatataatgcttatgcagagcaggtgtgacggat

SEQ ID NO: 424 - DNA, 72 bp, Artificial Sequence
  chemically synthesized
atccgtcacacctgctctgcataagcattatatgtagtaggagtttcgttggcctggtgttggctcccgtat

SEQ ID NO: 425 - DNA, 72 bp, Artificial Sequence; identical to SEQ ID NO: 417
  chemically synthesized

SEQ ID NO: 426 - DNA, 72 bp, Artificial Sequence; identical to SEQ ID NO: 418
  chemically synthesized